SEQUENCE LISTING (55 sequences)

SEQ ID NO: 1 - DNA, 130 bp, Adeno-associated virus
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 2000 bp, Artificial Sequence
  Description of Artificial Sequence: Synthetic polynucleotide
  ProA18 promoter
attgaagacctcagactttagagataccagagctatgggatacctgctgagaaaagctgctaacagggagtggaaccagaccaggaaaaagaagtttgttacagtcaacaaagatgaatggaattggagatctgatgagcactctgacattagaaatggagatgcagagtttggagtttgcctagctcttttttggggtgtggggtggggtgggtccttgtttgctccagtatttcctcacaatgacaatttagaatgatggtgtatacactgcggtatttgaggtatgtgatctgctttttgattttgactttataggagattacagataagtgatcaaatgaaactcagaaaagactttgacctttagacttttaacattattgagaatgccatagactatggagacttttgaagtggggactaaatttattttgcatcatgctttggctaggtatggcctccatagactcatctgttcgaacaagcctaagggagccaaggggtggaatgtggtggtttgaatatgcttggcccaggaaggacactattaggaggtatggacttgctggaagaagcttgtcactgtgggagtgggctttgagatcctttttctaccttgatgataatggaccaaacgtctgaacctgtaaaccagtcccaattaaatgttttcttttataagagttgccttggtcatggtgtatactcacagcaatgtaaactctaagatgggggacaatgggaggtgccagggcctacatgatagaaggacaggcattgttacaattccacctaccactcactacatggtcttggactgatttgcaaccaacttctctccaatgtcctcctcggaaatagagatgtcctggcatctcacccatggggttatatttaggaggcttttgatgatcatccactagttcagtctaatatctactactttaatagacatgagttcctttgctaataaccctctgggatttagtttctccatctgaaaattagttgtcctgtggctcattgttttctcgtgagaatttcagcatgagccagtacaaaagttgtaccaagaccttgtgtgtagaagccgaagttcttagtgggtcatgaggtactttcaaaaagaatgcaagccattctttatcctgagagatattttatgattgcatagctcaatggctgtctgtgagacaggaagtgaagccctaaatccatgatggagttcaaccagcacttaactagggaagggcatgaagcagaaatgactcagttgacaggaaaaccatccaatggcagcagtgcagagcagacagccagtcatggcagactcagtaccagaggtcaagggtcaggtactagtcaacatttgctttatgacagcacgtaactttacaaacctcaccctgcccaccaaatgcttgcctacacatacttctgagcctgtgaatgaacatacaacacacacccacacacatgcaaatgcacgcgcacacacacacacacactcacacacacacacacacacacagagagagagagagagagagagagagagagagagagagagagagagatgcacacacacaggcagcttgtcagcagctgtgcacatacaaggatctggctatccaattctcggggacagctgcagctcaaatcctttcttccacttccctcccttagttatgcaacccttacccaattcagctttcactcacacaccatttggatccaagaccttaatcctgcctagtgggctggaatgaacaagacagagctcattccagcttcacaaaagctgcactatccatctactgaatggattctttctatgtgagccaagaggaagacttagaaggataagaaataaaaaaggtgttattagtctaccataataatctccacatgccagcaagggagtgaccatttaaaaggagagacctagcttcagagagccagaaaagagctgtgtagctgacagagggagtccaggg

SEQ ID NO: 3 - DNA, 1317 bp, Artificial Sequence
  Description of Artificial Sequence: Synthetic polynucleotide
  ProB4 promoter
gttctttgggttccatattttcctcattccccttcaagtctagctggattgcaaagttaagcttatgattatgaataaaaactattgagtatattttcaaggaaatagtgacttaccttttaaggtcacttcctgctctttcatctgacccagaaacactgaataataaagccatcatttgttgaatgaccactgtgtaatttttatttagaactctaatgttggtaatctgcttggggctgtgggttaagaatctttgggttgcttgaggaaatgcacttaattaaaataaacattaaacttttccacactggcctttgtctccccctggcggatcctagcaacagataccagcaagttgtcttacttggtatttagggcaatgtgatgcagccctaacccaaagtgttgtcacaccaaacccttcacacccaagaggatgtttcgctctgaagtcatcagctctaacaaatatttgtaagtaaacaggtttttctttaaggaggccttcatttgtaaccaacaccatgctctcctttttcgctctctgaagtcatcagctctaaaggaaatcaatctcaatgccctattataccctgatctctggcctttggaggggctgttcaaagattctctgaggacttataggctaataaggttgaggattttgtgatttaaagaactccttaatccactgcttcctgcctaccataagccaaggccacccttgcctttttttttcttatcccctgctgaacctgaaaaaccctctcttttctacagttttctgttcccaggccccaagtacagttaaacaattcaactcaacttgtttaatcctctcgtcttaaattattcacattgccaagtatttcaacagaatgtactggcaagttcataaacagagtgcaaatcacccaagcagaagtaatttactgcttttaagtcaaagccctgcttagctagggagtcagctgtaggaattaaccagaacaaaatcctgattaaacagctaatcggttctactccgctaacccagtggcctgagcaaatcacaaggaggattggttccttgcatctggtttgggcaccgcccacctggaaccctaggggcggggccgcttaagtggcaggagggatatatatcaggatgcaaaagaccctgccaactggaaaaataagaaggcagcagctttggattttccactcatggaagaggggaggaaagggcagagggtgggggcttccttgggaagcttgggatgagtcaccacgggattggggagtggagggtggataggatatggagaagggtgg

SEQ ID NO: 4-8 - skipped (5 entries)

SEQ ID NO: 9 - DNA, 272 bp, Homo sapiens
ttcgaacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggtt

SEQ ID NO: 10 - DNA, 183 bp, Simian virus 40
aactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatcc

SEQ ID NO: 11 - DNA, 493 bp, Homo sapiens
cgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggat

SEQ ID NO: 12 - DNA, 6 bp, Unknown
  Description of Unknown: Kozak sequence
gccacc

SEQ ID NO: 13 - DNA, 1578 bp, Homo sapiens
atggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctaa

SEQ ID NO: 14 - DNA, 1578 bp, Unknown
  Description of Unknown: Codon-optimized CYP4V2 sequence
atggccggactgtggctgggactggtctggcagaagttattactgtggggagctgcctccgctctgtctttagctggagcttctttagtgctgtctttactgcagagggtcgcctcctatgctaggaagtggcagcagatgaggcctattcctaccgtggccagagcctatcctttagtgggccacgctctgctgatgaaacccgacggaagggagttcttccagcagatcatcgagtacaccgaagagtacagacacatgcctttactgaaactgtgggtgggacccgttcctatggtggctttatacaatgccgagaatgtggaggtgattttaaccagcagcaagcagatcgacaagtccagcatgtataagtttttagagccttggctcggtttaggactgctgacctccactggtaataagtggaggtctcgtaggaaaatgctgacccccacctttcacttcaccattttagaggactttttagatatcatgaacgagcaagctaacattttagtgaagaagctcgaaaagcacattaaccaagaagctttcaactgttttttctacatcactttatgcgccctcgatatcatctgcgagacagccatgggcaagaacattggcgctcagagcaacgacgattccgagtacgtgagggctgtctacagaatgagcgagatgatcttcagaagaatcaagatgccttggctgtggctggacctctggtatttaatgtttaaggaaggctgggagcataagaagtctttacagattttacatacatttaccaacagcgtgatcgccgagagggccaatgaaatgaacgccaacgaggattgtcgtggcgacggaagaggctccgctccttccaagaacaagaggagagcctttttagatctcttattatccgtgacagacgatgagggcaataggctgagccacgaggacatcagagaagaggtggacaccttcatgttcgagggacacgacacaaccgccgccgccatcaattggtctttatatttactcggcagcaaccccgaggtgcaaaaaaaggtcgaccacgagctcgacgacgtgttcggcaagagcgatcgtcccgccacagtggaagatttaaagaagctgaggtatctcgagtgcgtgatcaaagagactttaagactgttccccagcgtgcctctgtttgctcgttccgtgtccgaagactgcgaggtggctggatatcgtgtcctcaagggcaccgaggccgtgatcattccctacgccctccatcgtgatcccagatacttccccaatcccgaggagttccagcccgaaaggttcttccccgaaaacgctcaaggcagacacccttacgcttacgtgcctttctccgccggccctcgtaactgcattggccagaaattcgccgtcatggaggaaaagaccattttatcttgtattttaaggcacttctggatcgaaagcaatcagaaaagggaggaactcggtttagaaggacagctgattttaagacccagcaacggcatttggatcaagctgaagaggaggaacgccgacgagaggtga

SEQ ID NO: 15 - protein, 525 aa, Homo sapiens
MAGLWLGLVWQKLLLWGAASALSLAGASLVLSLLQRVASYARKWQQMRPIPTVARAYPLVGHALLMKPDGREFFQQIIEYTEEYRHMPLLKLWVGPVPMVALYNAENVEVILTSSKQIDKSSMYKFLEPWLGLGLLTSTGNKWRSRRKMLTPTFHFTILEDFLDIMNEQANILVKKLEKHINQEAFNCFFYITLCALDIICETAMGKNIGAQSNDDSEYVRAVYRMSEMIFRRIKMPWLWLDLWYLMFKEGWEHKKSLQILHTFTNSVIAERANEMNANEDCRGDGRGSAPSKNKRRAFLDLLLSVTDDEGNRLSHEDIREEVDTFMFEGHDTTAAAINWSLYLLGSNPEVQKKVDHELDDVFGKSDRPATVEDLKKLRYLECVIKETLRLFPSVPLFARSVSEDCEVAGYRVLKGTEAVIIPYALHRDPRYFPNPEEFQPERFFPENAQGRHPYAYVPFSAGPRNCIGQKFAVMEEKTILSCILRHFWIESNQKREELGLEGQLILRPSNGIWIKLKRRNADER

SEQ ID NO: 16 - DNA, 730 bp, Hepatitis B virus
taaacaggcctattgattggaaagtatgtcaacgaattgtgggtcttttggggtttgctgccccttttacgcaatgtggatatcctgctttaatgcctttatatgcatgtatacaagcaaaacaggcttttactttctcgccaacttacaaggcctttctaagtaaacagtatctgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcgaaactcatcgggactgacaattctgtcgtgctctcccgcaagtatacatcgtttccagggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcaactcga

SEQ ID NO: 17 - DNA, 588 bp, Woodchuck hepatitis virus
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 18 - DNA, 245 bp, Bos sp.
gatctggatgatgacgacaagtgaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagaattca

SEQ ID NO: 19 - DNA, 236 bp, Simian virus 40
gatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 20 - DNA, 2454 bp, Unknown
  Description of Unknown: SYNUCLEIN INTRONIC SEQUENCE AS STUFFER SEQUENCE
gggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatattttttttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaaaaggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagagtaaaatttctgttgattacagtaaaatattttgaccttaaattatgttgattacaatattcctttgataattcagagtgcatttcaggaaacacccttggacagtcagtaaattgtttattgtatttatctttgtattgttatggtatagctatttgtacaaatattattgtgcaattattacatttctgattatattattcatttggcctaaatttaccaagaatttgaacaagtcaattaggtttacaatcaagaaatatcaaaaatgatgaaaaggatgataatcatcatcagatgttgaggaagatgacgatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatatgatcggaaagcattaaacacaatagtgcctatacaaataaaatgttcctatcactgacttctaaaatggaaatgaggacaatgatatgggaatcttaatacagtgttgtggataggactaaaaacacaggagtcagatcttcttggttcaacttcctgcttactccttaccagctgtgtgttttttgcaaggttcttcacctctatgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatggcttctagggacctcctataaatatgatattgtgaagcatgattataataagaactagataacagacaggtggagactccactatctgaagagggtcaacctagatgaatggtgttccatttagtagttgaggaagaacccatgaggtttagaaagcagacaagcatgtggcaagttctggagtcagtggtaaaaattaaagaacccaactattactgtcacctaatgatctaatggagactgtggagatgggctgcatttttttaatcttctccagaatgccaaaatgtaaacacatatctgtgtgtgtgtgtgtgtgtgtgtgtgtgtgtgagagagagagagagagagagagagactgaagtttgtacaattagacattttataaaatgttttctgaaggacagtggctcacaatcttaagtttctaacattgtacaatgttgggagactttgtatactttattttctctttagcatattaaggaatctgagatgtcctacagtaaagaaatttgcattacatagttaaaatcagggttattcaaactttttgattattgaaacctttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattggg

SEQ ID NO: 21 - DNA, 1503 bp, Unknown
  Description of Unknown: RLBP1 INTRONIC SEQUENCE AS STUFFER SEQUENCE
attctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctgg

SEQ ID NO: 22 - DNA, 130 bp, Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 2217 bp, Adeno-associated virus
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 24 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 25 - protein, 601 aa, Adeno-associated virus
MAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 26 - protein, 535 aa, Adeno-associated virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - DNA, 2211 bp, Adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 28 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 599 aa, Adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 534 aa, Adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 32 - protein, 735 aa, Adeno-associated virus [sequence omitted]

SEQ ID NO: 33 - protein, 598 aa, Adeno-associated virus
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 34 - protein, 533 aa, Adeno-associated virus
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - DNA, 2175 bp, Adeno-associated virus
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 36 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 37 - protein, 588 aa, Adeno-associated virus
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 38 - protein, 532 aa, Adeno-associated virus
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 39 - DNA, 1578 bp, Macaca mulatta
atggcgggcatctggctggggctcgtgtggcagaagctgctgctgtggggcgcggcgagtgccgtgtccctggccggcgccagtctggtcctgagcctgctgcagagggtggcgagctacgtgaggaaatggcagcagatgcggcccatccccacggtggcccgcgcctacccactggtgggccacgcgctgctgatgaagcgggacgggcgagaattttttcagcagatcattgagtacacagaggaataccgccacatgccactcctgaagctctgggtcgggccggtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacatgttaaccaagaagcatttaactgctttgtttacatcactctttgtgccttagatatcatctgtgaaacagctatggggaagaatattggtgctcaaagtaacgatgattccgagtatgtccgtgcagtttatagaatgagtgagatgatatttcgaagaataaagatgccgtggctttggcttgacctctggtaccttatgtttaaagagggatgggaacacaaaaagagccttaagatcctacatgcttttaccaacaatgttatcgctgaacgggccaatgaaatgaacgtggatgaagactgtagaggtgatggcagggactccgccccctccaaaaataaacgcagggcctttcttgacttgcttttaagtgtgactgacgacgaagggaacaggctaagtcatgaagatattcgagaagaagttgacaccttcatgtttgagggccacgacacaactgcagctgcaatgaactggtccttatacctgttggggtctaacccagaagtccagaaaaaagtggaccatgaactggatgacgtgtttgggaggtctgaccgtcccgctactgtagaagacctgaagaaacttcggtatctggaatgtgttattaaggagacccttcgcctttttccttctgttcctttatttgcccgcagtgttagtgaagattgtgaagtggcaggttacagagttctgaaaggcactgaagccgtcatcattccctatgcattgcatagagatccaagatacttccccaaccctgaggagttccggcctgagcggttcttccccgagaatgcacaagggcgccatccatatgcctacgtgcccttctctgctggccccaggaactgtataggtcaaaagtttgctgtgatggaagaaaagaccattctttcgtgcatcctaaggcacttttggatagaatccaaccagaaaagagaagaacttggtctagaaggacagttgattcttcgtccaactaatggcatctggatcaagttgaagaggagaaatgcagatgaaccctaa

SEQ ID NO: 40 - protein, 525 aa, Macaca mulatta
MAGIWLGLVWQKLLLWGAASAVSLAGASLVLSLLQRVASYVRKWQQMRPIPTVARAYPLVGHALLMKRDGREFFQQIIEYTEEYRHMPLLKLWVGPVPMVALYNAENVEVILTSSKQIDKSSMYKFLEPWLGLGLLTSTGNKWRSRRKMLTPTFHFTILEDFLDIMNEQANILVKKLEKHVNQEAFNCFVYITLCALDIICETAMGKNIGAQSNDDSEYVRAVYRMSEMIFRRIKMPWLWLDLWYLMFKEGWEHKKSLKILHAFTNNVIAERANEMNVDEDCRGDGRDSAPSKNKRRAFLDLLLSVTDDEGNRLSHEDIREEVDTFMFEGHDTTAAAMNWSLYLLGSNPEVQKKVDHELDDVFGRSDRPATVEDLKKLRYLECVIKETLRLFPSVPLFARSVSEDCEVAGYRVLKGTEAVIIPYALHRDPRYFPNPEEFRPERFFPENAQGRHPYAYVPFSAGPRNCIGQKFAVMEEKTILSCILRHFWIESNQKREELGLEGQLILRPTNGIWIKLKRRNADEP

SEQ ID NO: 41 - DNA, 1584 bp, Bos taurus
atgctggcgccgtggttgctgagcgtcgggccgaagctgctgctctggagcgggctgtgcgccgtctccctggcaggcgccaccctcaccctgaacctcctgaagatggtggcgagctatgcgcggaaatggcgtcagatgcgtcccgtcccgaccattggggacccctaccccttggtgggacacgcgctgatgatgaagcccgatgcaagagatttttttcagcagataattgatttcactgaagaatgccgacacctgccactgctgaaactctggctcgggcctgtgcctctcgtggccctttataacgcagaaactgtggaagtaattttaagcagttcaaagcacattgaaaaatcctatatgtacaagttcttagaaccgtggcttggactaggacttcttacaagtactggaaacaaatggcgatctaggagaaaaatgttaacacccactttccattttacaattctggaggatttcttagatgtcatgaatgaacaagcaaatatattggttactaagcttgaaaagcatgttaaccaagaagcgtttaactgctttttttacgtcactctttgtaccttagatataatctgtgaaacagctatgggaaagaacattggtgctcaaagaaatgatgattccgagtatgttcgagccgtttataggatgagtgattcgatacatcaaagaatgaagatgccctggctctggcttgaccttatattctatatgtttaaaaatggacgagaacacagaaggagcctaaagattgtacatgattttaccaacaatgtcatcactgaacgggccaatgaaatgaagagacatgaagaaggtacgagtaacgacaaggagaaggactttcctccacgcaaaactaaatgcagggcttttcttgacttgcttttaaatgtgactgatgaccaagggaacaagctgagtcatgaagatataagagaagaagtcgacacctttatgtttgagggccatgatacaactgcagctgcaataaactggtccttgtatctgttgggttggtatccagaagtccagcagagagtggacactgagctggaagaagtgtttgggaagtctgaccgtcctgttaccctagaagacctgaagaaacttaaatatctggactgtgttattaaggagagccttcgccttttcccgtctgttcctttctttgcccgtaatcttaccgaagactgtgaagttgcgggtcacaaaatcgtgcaaggctgtcaagtaatcattgtgccctacgcactgcatagagatccaaagtacttcccggatcctgaggaattcaagccagaacggttctttcccgagaatttgaaaggacgtcatacatacgcatatgtgcccttctctgcaggcccccgaaactgtataggtcaaaagtttgccataatggaagaaaagaccattctttcctgcatccttaggcacttttgggtagaatccaaccaaaaaagagaagaacttggtctagcaggagagctcattcttcgtccaagtaacggcatctggatcaagttgaagaggagaaacacagatgaatcctaa

SEQ ID NO: 42 - protein, 527 aa, Bos taurus
MLAPWLLSVGPKLLLWSGLCAVSLAGATLTLNLLKMVASYARKWRQMRPVPTIGDPYPLVGHALMMKPDARDFFQQIIDFTEECRHLPLLKLWLGPVPLVALYNAETVEVILSSSKHIEKSYMYKFLEPWLGLGLLTSTGNKWRSRRKMLTPTFHFTILEDFLDVMNEQANILVTKLEKHVNQEAFNCFFYVTLCTLDIICETAMGKNIGAQRNDDSEYVRAVYRMSDSIHQRMKMPWLWLDLIFYMFKNGREHRRSLKIVHDFTNNVITERANEMKRHEEGTSNDKEKDFPPRKTKCRAFLDLLLNVTDDQGNKLSHEDIREEVDTFMFEGHDTTAAAINWSLYLLGWYPEVQQRVDTELEEVFGKSDRPVTLEDLKKLKYLDCVIKESLRLFPSVPFFARNLTEDCEVAGHKIVQGCQVIIVPYALHRDPKYFPDPEEFKPERFFPENLKGRHTYAYVPFSAGPRNCIGQKFAIMEEKTILSCILRHFWVESNQKREELGLAGELILRPSNGIWIKLKRRNTDES

SEQ ID NO: 43 - DNA, 1578 bp, Rattus norvegicus
atgttgtggctgtggttagggctcagcgggcagaagctattgctttggggcgcagcgagcgcggtctccgtggccggcgccactgtcttgctcaacatcctgcagatgttggtaagctatgcacgaaagtggcagcagatgcggccaatcccgtcggtggctcgcgcttaccccttggtgggacatgcgctgtttatgaagcccaacaacacagaattttttcagcagataattcagtacacagaagaattccgacacctgcccatcattaaactctggattggaccagtgcccctggtggcactttataaggcagagaatgtggaggtgattttgaccagttcgaagcaaattgataaatcctttatgtacaagttcctacagccatggctgggactaggacttcttacaagtactgggagcaaatggcgtgccaggaggaagatgttaacacccagtttccattttacaattctggaggatttcttagatgtcatgaatgagcaagcaaatatattggttaacaagcttgaaaaacatgtcaatcaagaggcctttaactgctttttccccatcactctttgtgctctggatataatctgtgaaacggctatggggaagaacattggagctcaaagtaatggtgattctgagtatgtccgtacagtgtataggatgagcgatatgatatacagaagaatgaagatgccctggttttggtttgacctttggtaccttatgtttaaagaaggaagggaccacaaaaagggactaaagagtctacatacttttaccaacaatgtcattgctgaacgggttaatgcaaggaaggcagagcaagactgcataggtgctgggaggggtcctctcccctcgaaaactaagcgcaaggcctttcttgacttgcttttgagtgtgactgatgaggaaggaaacaaattaagccatgaagacatccgagaggaagttgacaccttcatgtttgagggtcacgatacaactgctgctgccataaactggtccttatacctcctgggctctaatccagaagtccagaggaaagtggacaaggagctggatgatgtgtttggaagatcccatcgccctgtcaccttggaagacctgaagaaacttaaatatctggattgtgtcattaaggagaccctccgtgttttcccatctgtccctttatttgcccggagtcttagcgaagactgtgaagtggcgggttacaaaatctcaaaaggaacggaagcagtcatcattccctatgcactacatcgagaccctagatacttcccagaccctgaggaattccagccagagcggttctttcctgaaaactcccaaggacgccacccctatgcctatgtgccattctctgctggacctagaaactgcattggtcaaaagtttgcggtcatggaggaaaagaccattcttgcctgtatcctgagggagttttggatagaatccaaccagaagagagaagaactcggcctggctggagatttgattcttaggccaaataatggcatctggatcaagctgaagaggaggcatgaagatgacccctaa

SEQ ID NO: 44 - protein, 525 aa, Rattus norvegicus
MLWLWLGLSGQKLLLWGAASAVSVAGATVLLNILQMLVSYARKWQQMRPIPSVARAYPLVGHALFMKPNNTEFFQQIIQYTEEFRHLPIIKLWIGPVPLVALYKAENVEVILTSSKQIDKSFMYKFLQPWLGLGLLTSTGSKWRARRKMLTPSFHFTILEDFLDVMNEQANILVNKLEKHVNQEAFNCFFPITLCALDIICETAMGKNIGAQSNGDSEYVRTVYRMSDMIYRRMKMPWFWFDLWYLMFKEGRDHKKGLKSLHTFTNNVIAERVNARKAEQDCIGAGRGPLPSKTKRKAFLDLLLSVTDEEGNKLSHEDIREEVDTFMFEGHDTTAAAINWSLYLLGSNPEVQRKVDKELDDVFGRSHRPVTLEDLKKLKYLDCVIKETLRVFPSVPLFARSLSEDCEVAGYKISKGTEAVIIPYALHRDPRYFPDPEEFQPERFFPENSQGRHPYAYVPFSAGPRNCIGQKFAVMEEKTILACILREFWIESNQKREELGLAGDLILRPNNGIWIKLKRRHEDDP

SEQ ID NO: 45 - DNA, 1578 bp, Mus musculus
atgttgtggctgtggttagggctcagtgggcagaaactattgctttggggcgcagcgagcgcggtctccctggccggcgccactatcctgatcagcatctttccgatgctggtaagctacgcgcggaaatggcagcagatgcggtcaatcccgtcggtggcccgcgcctaccccttggtgggacacgcgctttatatgaagcccaacaacgcagaattttttcagcagctaatttattacacagaagaatttcgacacctgccgatcattaaactttggattggacccgtgcccctggtggcactttataaggcagagaatgtggaggtgattttgaccagttctaagcaaattgataaatcgtttttgtacaagttcctacagccatggctgggactaggacttcttacaagtacggggagcaaatggcgcaccaggaggaagatgctaacgcccactttccattttaccattctggagaacttcttggatgtcatgaatgagcaagcaaatatattggttaataagcttgaaaaacacgtcaaccaagaagcctttaattgttttttttacatcactctttgtgctctggatataatctgtgaaacggctatggggaagaacatcggagctcaaagcaataatgattccgagtatgtccgtacagtgtataggatgagtgatatgatatatagaagaatgaagatgccctggctttggtttgacctttggtaccttgtgtttaaagaaggacgggaccacaaaaggggactcaaatgcctacatactttcaccaacaatgtcattgctgaacgagtcaaagaaaggaaggcagaggaagactggacgggtgctggcaggggtcctatcccctccaaaaataagcgcaaggctttccttgacttgcttttgagtgtgactgatgaggaaggaaacagattaagccaggaagacatccgagaggaagttgacaccttcatgtttgagggtcacgatacaactgctgctgcaatcaactggtccttatacctattgggcacgaatccagaagtccagaggaaagtggatcaggagctggatgaagtgtttggaagatcccatcgtcctgtcaccttggaagacctgaagaaacttaaatatttggattgcgtcattaaggagactctccgagttttcccatctgtccctttatttgcccggagtcttagcgaggactgtgaagtgggcggttacaaagtcacaaaaggaacggaagcaatcatcattccctacgcactacaccgagaccccagatacttcccagatccagaggaattccgaccagagcggttctttcctgaaaattcccaaggacgccatccctatgcctatgtgccattttctgctggacctcgaaactgtattggtcaaaagtttgctgtcatggaggagaagaccattcttgcctgtatcctgaggcagttttgggtagaatccaaccagaagagagaagaactcggcctggctggagatttgattcttaggccaaataatggcatctggatcaagctgaagaggagacatgaagatgacccctaa

SEQ ID NO: 46 - protein, 525 aa, Mus musculus
MLWLWLGLSGQKLLLWGAASAVSLAGATILISIFPMLVSYARKWQQMRSIPSVARAYPLVGHALYMKPNNAEFFQQLIYYTEEFRHLPIIKLWIGPVPLVALYKAENVEVILTSSKQIDKSFLYKFLQPWLGLGLLTSTGSKWRTRRKMLTPTFHFTILENFLDVMNEQANILVNKLEKHVNQEAFNCFFYITLCALDIICETAMGKNIGAQSNNDSEYVRTVYRMSDMIYRRMKMPWLWFDLWYLVFKEGRDHKRGLKCLHTFTNNVIAERVKERKAEEDWTGAGRGPIPSKNKRKAFLDLLLSVTDEEGNRLSQEDIREEVDTFMFEGHDTTAAAINWSLYLLGTNPEVQRKVDQELDEVFGRSHRPVTLEDLKKLKYLDCVIKETLRVFPSVPLFARSLSEDCEVGGYKVTKGTEAIIIPYALHRDPRYFPDPEEFRPERFFPENSQGRHPYAYVPFSAGPRNCIGQKFAVMEEKTILACILRQFWVESNQKREELGLAGDLILRPNNGIWIKLKRRHEDDP

SEQ ID NO: 47 - DNA, 1593 bp, Gallus gallus
atggcaatggagatcacgctaggatccatggagggaacacagctgctgccctgggtggctggagccatcaccctgctgctgacggtggtgactgtacacttcctaccctctttgctgaactactggtggtggtggtgggtgatgaagcccatcccaggcatccgcccatgctacccctttgtgggaaatgctctcctgttggagcgaaatggagaaggtttttttaaacagctacaacagtatgctgatgagttcaggaaaatgccaatgttcaaactctggttaggtccactgcctgtcacagtattgttccatcctgatagtgtggaggttattctgagcagttcaaagcatattaaaaaatcattcctgtacacatttctgcacccatggctggggactggacttttgacaagcactggagacaagtggcggtcacggaggaagatgataactcctacattccactttgcaatcttaaatgactttcttgaggttatgaatgaacaagggggtgttttgttggagaaacttgagaagcatgttgacaaggaaccatttaatatctttacagacatcactctgtgtgcacttgatattatctgtgaaactgcaatgggcaagaatctgggtgctcaagacaataaggattctgagtatgttcgtgctgtctacaggatgagtgatctaatccaacagcgacagaagagcccttggctttggcatgatcttatgtatcttctgttcaaggaaggaagagagcatgagcggaatcttaagattctgcatggttttacagatacggtaattgcagaaaaagttgcagaacttgaaaacaccaagctaacaaaacacgatactgacgtgaacactgaagaagaaagtggttccaaaaagagagaagctttcttagacatgctgctgaatgccacagatgatgaagggaaaaaactcagctacaaggacattcgtgaagaagtggatacttttatgtttgagggtcatgatacaacagcagctgctatgaactgggtcctatacttgcttggtcatcatcctgaagcccagaagaaggttcaccaagaactggatgaggtgtttggcaacacagagcgtcctgttacagtggatgatttgaagaaacttcgatacctcgagtgtgttgtgaaagaagccctgaggctcttcccttcagttcccatgttcgcccgttccttgcaagaggattgctacattagtggatataagctaccaaaaggcacgaatgtccttgtcttaacttatgtgctgcacagagatcctgagatcttccctgagccagatgaattcaggcctgagcgcttcttccctgaaaatagcaaaggaaggcacccatatgcttatgtgcccttctctgctggccccaggaactgcattggccaacgctttgcacaaatggaagagaaaactcttctagccctcatcctgcggcgcttttgggtggactgttctcaaaagccagaagagcttggtctgtcaggagaactaattcttcgtccaaataatggcatctgggttcaactgaagaggagaccaaaaactgtaacagaatga

SEQ ID NO: 48 - protein, 530 aa, Gallus gallus
MAMEITLGSMEGTQLLPWVAGAITLLLTVVTVHFLPSLLNYWWWWWVMKPIPGIRPCYPFVGNALLLERNGEGFFKQLQQYADEFRKMPMFKLWLGPLPVTVLFHPDSVEVILSSSKHIKKSFLYTFLHPWLGTGLLTSTGDKWRSRRKMITPTFHFAILNDFLEVMNEQGGVLLEKLEKHVDKEPFNIFTDITLCALDIICETAMGKNLGAQDNKDSEYVRAVYRMSDLIQQRQKSPWLWHDLMYLLFKEGREHERNLKILHGFTDTVIAEKVAELENTKLTKHDTDVNTEEESGSKKREAFLDMLLNATDDEGKKLSYKDIREEVDTFMFEGHDTTAAAMNWVLYLLGHHPEAQKKVHQELDEVFGNTERPVTVDDLKKLRYLECVVKEALRLFPSVPMFARSLQEDCYISGYKLPKGTNVLVLTYVLHRDPEIFPEPDEFRPERFFPENSKGRHPYAYVPFSAGPRNCIGQRFAQMEEKTLLALILRRFWVDCSQKPEELGLSGELILRPNNGIWVQLKRRPKTVTE

SEQ ID NO: 49 - DNA, 1134 bp, Canis lupus
atgttaacacccactttccattttacgattctggaagatttcttagatgtcatgaatgaacacgcaaatatattggttaataagcttgaaaaacatgttaaccaagaagcatttaactgctttttttacatcactctttgtgcattagatataatttgtgaaacagctatggggaagaatattggggctcaaaataatgaggattctgagtatgttcgtgccatctacagaatgagtgatacgatacatcgaagaatgaagatgccctggctctggcttgactttttgtttcttatgtttaaagaaggccgggaacacaaaaggaacctagagatcctacataattttaccaataatgtcatcactgaacgggccagtgaactgaagagagacgaagaacatggaagtgctgacaaggactgctccccctccaaaaataaacgcagagcttttcttgacttgcttttaaatgtgactgatgatgaagggaacaagctacgtcatgaagatgttcgagaagaagttgacaccttcatgtttgagggccatgatacgacagcagcggcgataaactggtccttatatctcttgggttcttacccagaagtccagaaacaagtggacagtgaactggaggacgtgtttgggaagtctgatcgtcctgctaccttagaagacctgaagaaactcaaatacctggagtgtgtcattaaggagagccttcgcctttttccttcagttcccttatttgcccgtaatcttaacgaagattgtgtagttgcgggttacaaggttgtgaaaggctcccaagcgatcatcattccctacgcacttcatagagatccaagatatttcccaaatcccgaggagttccagccagagcggttctttcctgaaaatttgcaaggacgccacccatatgcatacattcccttttctgctggacccagaaactgtataggtcaaaggtttgccataatggaagaaaagactgttctttcctgtgtcctgaggcatttttgggtagaatccaaccagaaaagagaagaacttggtctggcaggagagttgattcttcgtccaactaatggcatctggatcaagttgaagaggagaaatgcagatgaatcttaa

SEQ ID NO: 50 - protein, 377 aa, Canis lupus
MLTPTFHFTILEDFLDVMNEHANILVNKLEKHVNQEAFNCFFYITLCALDIICETAMGKNIGAQNNEDSEYVRAIYRMSDTIHRRMKMPWLWLDFLFLMFKEGREHKRNLEILHNFTNNVITERASELKRDEEHGSADKDCSPSKNKRRAFLDLLLNVTDDEGNKLRHEDVREEVDTFMFEGHDTTAAAINWSLYLLGSYPEVQKQVDSELEDVFGKSDRPATLEDLKKLKYLECVIKESLRLFPSVPLFARNLNEDCVVAGYKVVKGSQAIIIPYALHRDPRYFPNPEEFQPERFFPENLQGRHPYAYIPFSAGPRNCIGQRFAIMEEKTVLSCVLRHFWVESNQKREELGLAGELILRPTNGIWIKLKRRNADES

SEQ ID NO: 51 - DNA, 6 bp, Artificial Sequence
  Description of Artificial Sequence: Synthetic oligonucleotide
  Kozak sequence
gccgcc

SEQ ID NO: 52 - DNA, 6 bp, Artificial Sequence
  Description of Artificial Sequence: Synthetic oligonucleotide
  Kozak sequence
gacacc

SEQ ID NO: 53 - DNA, 6 bp, Artificial Sequence
  Description of Artificial Sequence: Synthetic oligonucleotide
  Kozak sequence
gccacg

SEQ ID NO: 54 - DNA, 103 bp, Artificial Sequence
  Description of Artificial Sequence: Synthetic polynucleotide
  Delta ITR
cgcgctcgctcgctcactgaggccgcccgggcaaagcccgggcgtcgggcgacctttggtcgcccggcctcagtgagcgagcgagcgcgcagagagggagtgg

SEQ ID NO: 55 - RNA, 13 nt, Unknown
  Description of Unknown: Kozak sequence
gccgccrccaugg

(19) United States
(12) Patent Application Publication
(10) Pub. No.: US 2022/0154211 A1
(43) Pub. Date: May 19, 2022

(54) COMPOSITIONS AND METHODS TO TREAT BIETTI CRYSTALLINE DYSTROPHY

(71) Applicants: NOVARTIS AG, Basel (CH); FRIEDRICH MIESCHER INSTITUTE FOR BIOMEDICAL RESEARCH, Basel (CH)

(72) Inventors: Christie L. BELL, Cambridge, MA (US); Josephine JUETTNER, Basel (CH); Jacek KROL, Basel (CH); Terri MCGEE, Walpole, MA (US); Botond ROSKA, Oberwil (CH)

(21) Appl. No.: 17/432,364

(22) PCT Filed: Feb. 24, 2020
(86) PCT No.: PCT/IB2020/051558

§ 371 Date: Aug. 19, 2021

Related U.S. Application Data
(60) Provisional application No. 62/810,257, filed on Feb. 25, 2019.

(51) Int. Cl. C12N 15/86; C12N 9/02; A61P 27/02; A61K 48/00

(52) U.S. Cl. CPC C12N 15/86; C12N 9/0071; C12Y 114/14; A61P 27/02; A61K 48/0058; C12N 2750/14171; C12N 2750/14143; C12N 2830/008; C12N 2830/48; C12N 2830/50; A61K 48/0075

(57) ABSTRACT

Viral vectors to deliver a heterologous CYP4V2 gene to the retina, e.g., RPE cells of the retina, are provided herein to treat subjects with Bietti crystalline dystrophy.

### CROSS-REFERENCE TO RELATED APPLICATION AND INCORPORATION OF SEQUENCE LISTING

This application claims benefit under 35 U.S.C. § 119(e) of U.S. Provisional Appln. No. 62/810,257, filed Feb. 25, 2019, herein incorporated by reference in its entirety. The sequence listing that is contained in the file named “PAT058468-WO-PCT SQL_ST25,” which is 156,133 bytes (measured in operating system MS-Windows) and was created on Feb. 22, 2020, is filed herewith and incorporated herein by reference.

### BACKGROUND

Bietti crystalline dystrophy (BCD) is an autosomal recessive disorder in which numerous small, yellow or white crystalline-like deposits of lipid accumulate in the retina, which is followed by chorioretinal atrophy and progressive vision loss. Subjects with BCD typically begin noticing vision problems in their teens or twenties. They often experience night blindness in addition to a reduction in visual acuity. They also usually lose areas of vision, most often peripheral vision. Color vision may also be impaired.

The vision problems may worsen at different rates in each eye, and the severity and progression of symptoms varies widely among affected subjects, even within the same family. However, most subjects with BCD become legally blind by 40 or 50 years of age. Most affected subjects retain some degree of vision, usually in the center of the visual field, although it is typically blurry and cannot be corrected by prescription lenses.

BCD is caused by mutations in the CYP4V2 gene. The gene, located on the long arm of human chromosome 4, encodes cytochrome P450 family 4 subfamily V member 2. As a member of the cytochrome P450 family of enzymes, the w-hydroxylase is involved in lipid metabolism, specifically oxidation of polyunsaturated fatty acids such as docosahexaenoic acid (DHA) and eicosapentaenoic acid (EPA). At least 80 different CYP4V2 gene mutations have been identified in subjects with BCD (Zhang et al., Mol Vis 24:700-711, 2018). CYP4V2 gene mutations that cause BCD impair or eliminate the function of the enzyme and are believed to affect lipid breakdown. However, it is unknown how they lead to the specific signs and symptoms of BCD.

BCD is estimated to affect approximately 65,000 people worldwide (Xiao et al., Biochem Biophys Res Comm 409:181-186, 2011; and Mataftsi et al., Retina 24:416-426, 2004). It is more common in people of East Asian descent, especially those of Chinese and Japanese background. Currently, there is no treatment available for BCD.

### SUMMARY

The present invention relates generally to recombinant viral vectors and methods of using recombinant viral vectors to express proteins in the retina, e.g., retinal pigment epithelium (RPE) cells, of subjects suffering from retinal diseases and blindness, e.g., BCD.

The present invention, in one aspect, relates to viral vectors that are capable of delivering a heterologous gene to the retina, e.g., human retina. The present invention also relates to viral vectors that are capable of directing a heterologous gene to the retina, e.g., RPE cells of the retina. The present invention further relates to viral vectors that are recombinant adeno-associated viral vectors (rAAV). In certain embodiments the rAAV viral vector may be selected from among any AAV serotype known in the art, including without limitation, AAV1 to AAV12. In certain embodiments, the rAAV vector capsid is an AAV8 serotype. In certain other embodiments, the rAAV vector capsid is an AAV9 serotype. In certain embodiments, the rAAV vector capsid is an AAV2 serotype. In certain embodiments, the rAAV vector capsid is an AAV5 serotype. In certain embodiments, the rAAV vector is a novel synthetic AAV serotype derived from modified wild-type AAV capsid sequences.

In one aspect, viral vectors are provided, wherein the viral vectors comprise a vector genome comprising, in a 5′ to 3′ direction:

(i) a 5′ ITR;

(ii) a promoter;

(iii) a recombinant nucleotide sequence comprising a CYP4V2 coding sequence;

(iv) a polyadenylation (polyA) signal sequence; and

(v) a 3′ ITR.

In one embodiment, the vector genome comprises, in the 5′ to 3′ direction:

(i) a 5′ ITR;

(ii) a promoter;

(iii) an intron;

(iv) a recombinant nucleotide sequence comprising a CYP4V2 coding sequence;

(v) a polyA signal sequence; and

(vi) a 3′ ITR.

In some embodiments, the vector genome comprises, in the 5′ to 3′ direction:

(i) a 5′ ITR;

(ii) a promoter;

(iii) a recombinant nucleotide sequence comprising a CYP4V2 coding sequence;

(iv) a regulatory element;

(v) a polyA signal sequence; and

(vi) a 3′ ITR.

In one embodiment, the vector genome comprises, in the 5′ to 3′ direction:

(i) a 5′ ITR;

(ii) a promoter;

(iii) an intron;

(iv) a recombinant nucleotide sequence comprising a CYP4V2 coding sequence;

(v) a regulatory element;

(vi) a polyA signal sequence; and

(vii) a 3′ ITR.

In some embodiments, the vector genome comprises a length greater than or about 4.1 kb and less than or about 4.9 kb. In another embodiments, the vector genome comprises a length less than or about 5 kb.

In one embodiment, the vector genome comprises a stuffer sequence positioned between the polyA signal sequence and the 3′ ITR. In some embodiments, the stuffer sequence is between about 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, or 2,500-3,000 nucleotides in length.

In one embodiment, the 5′ ITR comprises a nucleotide sequence with greater than or about 90% identity to SEQ ID NO:1.

In one embodiment, the promoter is a retinal pigment epithelium (RPE)-specific promoter, e.g., a ProA18 promoter or ProB4 promoter, e.g., wherein the promoter comprises a nucleotide sequence with greater than or about 90% identity to SEQ ID NO:2 or SEQ ID NO:3, and promotes expression of the CYP4V2 preferentially in RPE cells.

The present invention hence provides an isolated nucleic acid molecule comprising, or consisting of, the nucleic acid sequence of SEQ ID NO: 2 or a nucleic acid sequence having at least 80%, at least 85%, at least 90%, at least 95%, at least 96%, at least 97%, at least 98%, or at least 99% identity to said nucleic acid sequence of SEQ ID NO: 2. The isolated nucleic acid of SEQ ID NO: 2 leads to the expression in human or NHP retinal cells, e.g., human or NHP RPE cells, of a gene operatively linked to the nucleic acid sequence of SEQ ID NO: 2.

The present invention hence provides an isolated nucleic acid molecule comprising, or consisting of, the nucleic acid sequence of SEQ ID NO: 3 or a nucleic acid sequence having at least 80%, at least 85%, at least 90%, at least 95%, at least 96%, at least 97%, at least 98%, or at least 99% identity to said nucleic acid sequence of SEQ ID NO: 3. The isolated nucleic acid of SEQ ID NO: 3 leads to the expression in human or NHP retinal cells, e.g., human or NHP RPE cells, of a gene operatively linked to the nucleic acid sequence of SEQ ID NO: 3.

In some embodiments, the CYP4V2 coding sequence comprises a nucleotide sequence with greater than or about 90% identity to SEQ ID NO:13, SEQ ID NO:14, SEQ ID NO:39, SEQ ID NO:41, SEQ ID NO:43, SEQ ID NO:45, SEQ ID NO:47, or SEQ ID NO:49.

In one embodiment, the polyA signal sequence comprises a bovine growth hormone or simian virus 40 polyA nucleotide sequence, e.g., wherein the polyA signal sequence comprises a nucleotide sequence with greater than or about 90% identity to SEQ ID NO:18 or SEQ ID NO:19.

In some embodiments, the 3′ ITR comprises a nucleotide sequence with greater than or about 90% identity to SEQ ID NO:22.

In one embodiment, the intron comprises a human growth hormone, simian virus 40, or human beta goblin intron sequence, e.g., wherein the intron comprises a nucleotide sequence with greater than or about 90% identity to SEQ ID NO:9, SEQ ID NO:10, or SEQ ID NO:11.

In some embodiments, the regulatory element comprises a hepatitis B virus or woodchuck hepatitis virus sequence, e.g., wherein the regulatory element comprises a nucleotide sequence with greater than or about 90% identity to SEQ ID NO:16 or SEQ ID NO:17.

In one embodiment, the vector genome comprises a Kozak sequence positioned immediately upstream of the recombinant nucleotide sequence comprising the CYP4V2 coding sequence, e.g., wherein the Kozak sequence comprises the nucleotide sequence of SEQ ID NO:12, SEQ ID NO:51, SEQ ID NO:52, or SEQ ID NO:53.

In some embodiments, the vector genome comprises, in the 5′ to 3′ direction, nucleotide sequences selected from the group consisting of:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22; and

viii) SEQ ID NOs:1, 3, 14, 19, and 22.

In one embodiment, the vector genome comprises, in the 5′ to 3′ direction, nucleotide sequences selected from the group consisting of:

i) SEQ ID NOs:1, 2, 9, 13, 18, and 22;

ii) SEQ ID NOs:1, 3, 9, 13, 18, and 22;

iii) SEQ ID NOs:1, 2, 9, 14, 18, and 22;

iv) SEQ ID NOs:1, 3, 9, 14, 18, and 22;

v) SEQ ID NOs:1, 2, 9, 13, 19, and 22;

vi) SEQ ID NOs:1, 3, 9, 13, 19, and 22;

vii) SEQ ID NOs:1, 2, 9, 14, 19, and 22; and

viii) SEQ ID NOs:1, 3, 9, 14, 19, and 22.

In some embodiments, the vector genome comprises, in the 5′ to 3′ direction, nucleotide sequences selected from the group consisting of:

i) SEQ ID NOs:1, 2, 13, 16, 18, and 22;

ii) SEQ ID NOs:1, 3, 13, 16, 18, and 22;

iii) SEQ ID NOs:1, 2, 14, 16, 18, and 22;

iv) SEQ ID NOs:1, 3, 14, 16, 18, and 22;

v) SEQ ID NOs:1, 2, 13, 16, 19, and 22;

vi) SEQ ID NOs:1, 3, 13, 16, 19, and 22;

vii) SEQ ID NOs:1, 2, 14, 16, 19, and 22; and

viii) SEQ ID NOs:1, 3, 14, 16, 19, and 22.

In one embodiment, the vector genome comprises, in the 5′ to 3′ direction, nucleotide sequences selected from the group consisting of:

i) SEQ ID NOs:1, 2, 9, 13, 16, 18, and 22;

ii) SEQ ID NOs:1, 3, 9, 13, 16, 18, and 22;

iii) SEQ ID NOs:1, 2, 9, 14, 16, 18, and 22;

iv) SEQ ID NOs:1, 3, 9, 14, 16, 18, and 22;

v) SEQ ID NOs:1, 2, 9, 13, 16, 19, and 22;

vi) SEQ ID NOs:1, 3, 9, 13, 16, 19, and 22;

vii) SEQ ID NOs:1, 2, 9, 14, 16, 19, and 22; and

viii) SEQ ID NOs:1, 3, 9, 14, 16, 19, and 22.

In some embodiments, the vector comprises an adeno-associated virus (AAV) serotype 8, 9, 2, or 5 capsid. In one embodiment, the AAV8 capsid comprises VP1, VP2, and VP3 amino acid sequences with greater than or about 90% identity to SEQ ID NOs:24, 25, and 26, respectively. In some embodiments, the AAV8 capsid is encoded by a nucleotide sequence with greater than or about 90% identity to SEQ ID NO:23. In one embodiment, the AAV9 capsid comprises VP1, VP2, and VP3 amino acid sequences with greater than or about 90% identity to SEQ ID NOs:28, 29, and 30, respectively. In some embodiments, the AAV9 capsid is encoded by a nucleotide sequence with greater than or about 90% identity to SEQ ID NO:27. In one embodiment, the AAV2 capsid comprises VP1, VP2, and VP3 amino acid sequences with greater than or about 90% identity to SEQ ID NOs:32, 33, and 34, respectively. In some embodiments, the AAV2 capsid is encoded by a nucleotide sequence with greater than or about 90% identity to SEQ ID NO:31. In one embodiment, the AAV5 capsid comprises VP1, VP2, and VP3 amino acid sequences with greater than or about 90% identity to SEQ ID NOs:36, 37, and 38, respectively. In some embodiments, the AAV5 capsid is encoded by a nucleotide sequence with greater than or about 90% identity to SEQ ID NO:35.

In another aspect, the present disclosure provides compositions comprising a viral vector described herein. In one embodiment, the compositions further comprise a pharmaceutically acceptable excipient. In some embodiments, the compositions are for use in treating a subject with BCD, e.g., for use in improving visual acuity in a subject with BCD.

Also provided herein is a method of expressing a heterologous CYP4V2 gene in a retinal cell, wherein the method comprises contacting the retinal cell with a viral vector described herein. In some embodiments, the retinal cell is a RPE cell.

In another aspect, a method of treating a subject with Bietti crystalline dystrophy (BCD) is provided, wherein the method comprises administering to the subject an effective amount of a composition comprising a viral vector described herein, e.g., wherein the composition further comprises a pharmaceutically acceptable excipient.

In yet another aspect, a method of improving visual acuity, improving visual function or functional vision, or inhibiting decline of visual function or functional vision in a subject with BCD is provided, wherein the method comprises administering to the subject an effective amount of a composition comprising a viral vector described herein, e.g., wherein the composition further comprises a pharmaceutically acceptable excipient.

In one aspect, a nucleic acid comprising a gene cassette is provided, wherein the gene cassette comprises, in the 5′ to 3′ direction:

(i) a 5′ ITR;

(ii) a promoter;

(iii) a recombinant nucleotide sequence comprising a CYP4V2 coding sequence;

(iv) a polyA signal sequence; and

(v) a 3′ ITR.

In one embodiment, the nucleic acid comprising the gene cassette is a plasmid.

In some embodiments, the gene cassette comprises, in the 5′ to 3′ direction, nucleotide sequences selected from the group consisting of:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

### Definitions

Unless otherwise defined, all technical and scientific terms used herein have the same meaning as commonly understood by those of ordinary skill in the art to which this invention pertains.

The term “capsid” refers to the protein coat of the virus or viral vector. The term “AAV capsid” refers to the protein coat of the adeno-associated virus (AAV), which is composed of a total of 60 subunits; each subunit is an amino acid sequence, which can be viral protein 1(VP1), VP2, or VP3 (Muzyczka N and Berns K I (2001) Chapter 69, Fields Virology. Lippincott Williams & Wilkins).

The term “gene cassette” refers to a manipulatable fragment of DNA carrying, and capable of expressing, one or more genes or coding sequences of interest, for example, between one or more sets of restriction sites, though straddling restriction sites are not required. A gene cassette, or a portion thereof, can be transferred from one DNA sequence (often in a plasmid vector) to another by cutting the fragment out using restriction enzymes and ligating it back into a new context, for example, into a new plasmid backbone.

The term “heterologous gene” or “heterologous nucleotide sequence” will typically refer to a gene or nucleotide sequence that is not naturally-occurring in the virus. Alternatively, a heterologous gene or heterologous nucleotide sequence may refer to a viral sequence that is placed into a non-naturally occurring environment (e.g., by association with a promoter with which it is not naturally associated in the virus).

The terms “inverted terminal repeat” or “ITR” refer to a stretch of nucleotide sequences that exist in adeno-associated viruses (AAV) and/or recombinant adeno-associated viral vectors (rAAV) that can form a T-shaped palindromic structure, which is required for completing wild-type AAV lytic and latent life cycles (Muzyczka N and Berns K I (2001) Chapter 69, Fields Virology. Lippincott Williams & Wilkins). In rAAV, these sequences play a functional role in genome packaging and in second-strand synthesis.

The term “operably linked” refers to a functional relationship between two or more polynucleotide (e.g., DNA) segments. Typically, the term refers to the functional relationship of a transcriptional regulatory sequence to a sequence to be transcribed. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribable sequence are contiguous to the transcribable sequence, i.e., they are cis-acting. However, some transcriptional regulatory sequences, such as enhancers, need not be physically contiguous or located in close proximity to the coding sequences whose transcription they enhance.

As used herein, the term “percent sequence identity” refers to the degree of identity between any given query sequence and a subject sequence. A subject sequence typically has a length that is from about 80 percent to 250 percent of the length of the query sequence, e.g., 82, 85, 87, 89, 90, 93, 95, 97, 99, 100, 105, 110, 115, or 120, 130, 140, 150, 160, 170, 180, 190, 200, 210, 220, 230, 240, or 250 percent of the length of the query sequence. To determine the percent identity of two nucleotide sequences, or of two amino acid sequences, the sequences are aligned for optimal comparison purposes (e.g., gaps can be introduced in one or both of a first and a second amino acid or nucleotide sequence for optimal alignment and non-homologous sequences can be disregarded for comparison purposes). The nucleotides or amino acid residues at corresponding nucleotide positions or amino acid positions are then compared. When a position in the first sequence is occupied by the same nucleotide or amino acid residue as the corresponding position in the second sequence, then the molecules are identical at that position (as used herein, nucleotide or amino acid “identity” is equivalent to nucleotide or amino acid “homology”). The percent identity between the two sequences is a function of the number of identical positions shared by the sequences, taking into account the number of gaps, and the length of each gap, which needs to be introduced for optimal alignment of the two sequences.

In another embodiment, the percent identity of two amino acid sequences can be assessed as a function of the conservation of amino acid residues within the same family of amino acids (e.g., positive charge, negative charge, polar and uncharged, hydrophobic) at corresponding positions in both amino acid sequences (e.g., the presence of an alanine residue in place of a valine residue at a specific position in both sequences shows a high level of conservation, but the presence of an arginine residue in place of an aspartate residue at a specific position in both sequences shows a low level of conservation). For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

The term “promoter” refers to a sequence that regulates transcription of an operably linked gene, or nucleotide sequence encoding a protein. Promoters provide the sequence sufficient to direct transcription, as well as, the recognition sites for RNA polymerase and other transcription factors required for efficient transcription and can direct cell specific expression. In addition to the sequence sufficient to direct transcription, a promoter sequence of the invention can also include sequences of other regulatory elements that are involved in modulating transcription (e.g., enhancers, minimal promoters, Kozak sequences, and introns). Examples of promoters known in the art and useful in the viral vectors described herein include RPE-specific promoters such as the ProA18 promoter (e.g., SEQ ID NO:2) and ProB4 promoter (e.g., SEQ ID NO:3). In addition, standard techniques are known in the art for creating functional promoters by mixing and matching known regulatory elements. “Truncated promoters” may also be generated from promoter fragments or by mixing and matching fragments of known regulatory elements.

The term “CYP4V2” refers to cytochrome P450 family 4 subfamily V member 2. The human CYP4V2 gene is found on chromosome 4 and has the nucleotide coding sequence as set out, for example, in SEQ ID NO:13. In one embodiment, a codon-optimized sequence of the human CYP4V2 gene can be used. One example of such a codon-optimized CYP4V2 gene has the nucleotide coding sequence as set out in SEQ ID NO:14. The “CYP4V2 gene product” is the protein encoded by a CYP4V2 gene. In one embodiment, an exemplary human CYP4V2 gene product has an amino acid sequence as set out in SEQ ID NO:15. In one embodiment, a CYP4V2 coding sequence encodes the amino acid sequence of SEQ ID NO:15 or a functional variant or fragment thereof. Examples of CYP4V2 coding sequences and CYP4V2 gene products from other species can be found in Table 2 (e.g., SEQ ID NOs:39-50). The term “CYP4V2 coding sequence” or “CYP4V2 GENE CDS” or “CYP4V2 CDS” refers to a nucleotide sequence that encodes a CYP4V2 gene product. One of skill in the art will understand that a CYP4V2 coding sequence may include any nucleotide sequence that encodes a CYP4V2 gene product or a functional variant or fragment thereof. In one embodiment, the CYP4V2 coding sequence encodes the amino acid sequence of SEQ ID NO:15, 40, 42, 44, 46, 48, 50, or a functional variant or fragment thereof. The CYP4V2 coding sequence may or may not include intervening regulatory elements (e.g., introns, enhancers, or other non-coding sequences).

The term “subject” includes human and non-human animals. Non-human animals include all vertebrates (e.g., mammals and non-mammals) such as, non-human primates (e.g., cynomolgus monkey), mice, rats, sheep, dogs, cows, chickens, amphibians, and reptiles. Except when noted, the terms “patient” or “subject” are used herein interchangeably.

As used herein, the term “treating” or “treatment” of any disease or disorder (e.g., BCD) refers to ameliorating the disease or disorder such as by slowing or arresting or reducing the development of the disease or at least one of the clinical symptoms thereof. “Treating” or “treatment” can also refer to alleviating or ameliorating at least one physical parameter, including those that may not be discernible by the subject. “Treating” or “treatment” can also refer to modulating the disease or disorder, either physically, (e.g., stabilization of a discernible symptom), physiologically, (e.g., stabilization of a physical parameter), or both. More specifically, “treatment” of BCD means any action that results in the improvement or preservation of visual function, functional vision, retinal anatomy, and/or Quality of Life in a subject having BCD. As used herein, “treatment” may mean any manner in which one or more of the symptoms of BCD are ameliorated or otherwise beneficially altered. As used herein, amelioration of the symptoms of BCD refers to any lessening, whether permanent or temporary, lasting or transient, that can be attributed to or associated with treatment by the compositions and methods of the present invention. “Preventing or “prevention” as used herein, refers to preventing or delaying the onset or development or progression of the disease or disorder. “Prevention” as it relates to BCD means any action that prevents or slows a worsening in visual function, functional vision, retinal anatomy, Quality of Life, and/or a BCD disease parameter, as described below, in a patient with BCD and at risk for said worsening. Methods for assessing treatment and/or prevention of disease are known in the art and described herein below.

The term “virus vector” or “viral vector” is intended to refer to a non-wild-type recombinant viral particle (e.g., a parvovirus, etc.) that functions as a gene delivery vehicle and which comprises a recombinant viral genome packaged within a viral (e.g., AAV) capsid. A specific type of virus vector may be a “recombinant adeno-associated virus vector”, or “rAAV vector”. The recombinant viral genome packaged in the viral vector is also referred to herein as the “vector genome”.

### DESCRIPTION OF DRAWINGS

FIG. 1 are photomicrographs showing ChR2d-eGFP expression in flatmounts of the posterior eyecup. Eyecups were isolated from PFA-fixed eyes, cut into petals, and analyzed for eGFP fluorescence.

FIG. 2A and FIG. 2B are graphs showing mRNA expression levels of ChR2d-eGFP as measured by ddPCR. Fold change in expression relative to TM073 is shown for both the (FIG. 2A) posterior eyecup and (FIG. 2B) neural retina. ChR2d-eGFP expression was normalized to Rab7 control expression for each sample.

### DETAILED DESCRIPTION

The present disclosure is based in part on the discovery that expression of CYP4V2 from recombinant adeno-associated viral vectors (rAAV) having a combination of selected promoter, AAV genome, and capsid serotype provides a potent and efficacious treatment for BCD, e.g., to subjects with a mutation in their CYP4V2 gene (Table 1). Accordingly, the present disclosure provides recombinant viral vectors that direct expression of the CYP4V2 coding sequence to the retina, viral vector compositions, plasmids useful for generating the viral vectors, methods of delivering a CYP4V2 coding sequence to the retina, methods of expressing a CYP4V2 coding sequence in RPE cells of the retina, and methods of use of such viral vectors.

TABLE 1
CYP4V2 mutations associated with BCD

| Nucleotide change | Polypeptide change |
|---|---|
| c.31C > T | p.Q11X |
| c.64C > G | p.L22V |
| c.65T > A | p.L22H |
| c.71T > C | p.L24P |
| c.130T > A | p.W44R |
| c.134A > C | p.Q45P |
| c.181G > A | p.G61S |
| c.197T > G | p.M66R |
| c.215 − 2A > G | Splicing acceptor |
| c.219T > A | p.F73L |
| c.237G > T | p.E79D |
| c.253C > T | p.R85C |
| c.254G > A | p.R85H |
| c.283G > A | p.G95R |
| c.328-1G > A | Exon3del |
| c.332T > C | p.I111T |
| c.335T > G | p.L112X-termination |
| c.367A > G | p.M123V |
| c.368T > G | p.M123R |
| c.400G > T | p.G134X-termination |
| c.413 + 2T > G | Mis-splicing-Splicing acceptor |
| c.677T > A | p.M226L |
| c.694C > T | p.R232X (substitution-nonsense) |
| c.732G > A | p.W244X |
| c.791del T | deletion |
| c.801 + 5G > A | Exon6del |
| c.802-9A > G | Altered splicing |
| c.802 − 8_810del17insGC | Mis-splicing-Splicing acceptor |
| c.838G > T | p.E280X |
| c.958C > T | p.R320X |
| c.992A > C | p.H331P |
| c.994G > A | p.D332N |
| c.1020G > A | p.W340X |
| c.1027T > G | p.Y343D |
| c.1061 − 1062insA | frameshift |
| c.1091 − 2A > G | Mis-splicing-Splicing acceptor |
| c.1168C > T | p.R390C |
| c.1169G > A | p.R390H |
| c.1187C > T | p.P396L |
| c.1198C > T | p.R400C |
| c.1199G > A | p.R400H |
| c.1216T > C | p.C406R |
| c.1278G > T | p.L426F |
| c.1400G > A | p.C467Y |
| c.1508G > A | p.G503E |

Except as otherwise indicated, standard methods known to those skilled in the art may be used for the construction of recombinant parvovirus and rAAV vectors, using recombinant plasmids carrying a viral gene cassette, packaging plasmids expressing the parvovirus rep and/or cap sequences, as well as transiently and stably transfected packaging cells. Such techniques are known to those skilled in the art. (e.g., Sambrook et al., MOLECULAR CLONING: A LABORATORY MANUAL 2nd Ed. (Cold Spring Harbor, N.Y., 1989); Choi et al., CURRENT PROTOCOLS IN MOLECULAR BIOLOGY (2007)).

When a viral vector expresses a particular protein or activity, it is not necessary that the relevant gene(s) be identical to the corresponding gene(s) found in nature or disclosed herein. So long as the protein is functional, it may be used in accordance with one aspect of the present invention. One of skill in the art could readily determine if a CYP4V2 coding sequence encodes a functional w-hydroxylase by detecting hydroxylase activity. Briefly, a protein of interest is mixed with fatty acids and other required factors and incubated to allow the hydroxylation reaction to occur. Then, the hydroxylated fatty acids can be measured by mass spectrometry. See, e.g., a functional assay, as described in Nakano et al., Drug Metab Dispos 37:2119-2122, 2009. Very high sequence identity with the natural protein, however, is generally preferred. For instance, large deletions (e.g., greater than about 50 amino acids) should generally be avoided according to certain embodiments of the invention. Therefore, skilled practitioners will appreciate that the present viral vector sequences can vary from the sequences described herein. In some embodiments, the viral nucleotide or amino acid sequence has greater than or about 80% identity to the sequences provided herein, e.g., greater than or about 85%, 90%, 91%, 92%, 93%, 94%, 95%, 96%, 97%, 98%, 99%, or 100% identity to the sequences provided herein.

In some embodiments, a sequence change is a conservative substitution. Such a change includes substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., Table III of US 20110201052; pages 13-15 “Biochemistry” 2nd Ed. Stryer ed (Stanford University); Henikoff et al., Proc Natl Acad Sci USA 89:10915-10919, 1992; Lei et al., J Biol Chem 270:11882-11886, 1995).

### Viral Vectors

The present invention is related to viral vectors that direct expression of a heterologous gene to the retina, e.g., human retina. In certain aspects of the invention, expression is directed preferentially to RPE cells of the retina. A variety of viral vectors known in the art may be adapted by one of skill in the art for use in the present invention, for example, recombinant adeno-associated viruses, recombinant adenoviruses, recombinant retroviruses, recombinant poxviruses, and recombinant baculoviruses.

In particular, it is contemplated that the viral vector of the invention may be a recombinant adeno-associated (rAAV) vector. AAVs are small, single-stranded DNA viruses that require helper virus to facilitate efficient replication (Muzyczka N and Berns K I (2001) Chapter 69, Fields Virology. Lippincott Williams & Wilkins). The viral vector comprises a vector genome and a protein capsid. The viral vector capsid may be supplied from any of the AAV serotypes known in the art, including presently identified human and non-human AAV serotypes and AAV serotypes yet to be identified (see, e.g., Choi et al., Curr Gene Ther 5:299-310, 2005; Schmidt et al., J Virol 82:1399-1406, 2008; U.S. Pat. Nos. 9,193,956; 9,186,419; 8,632,764; 8,663,624; 8,927,514; 8,628,966; 8,263,396; 8,734,809; 8,889,641; 8,632,764; 8,691,948; 8,299,295; 8,802,440; 8,445,267; 8,906,307; 8,574,583; 8,067,015; 7,588,772; 7,867,484; 8,163,543; 8,283,151; 8,999,678; 7,892,809; 7,906,111; 7,259,151; 7,629,322; 7,220,577; 8,802,080; 7,198,951; 8,318,480; 8,962,332; 7,790,449; 7,282,199; 8,906,675; 8,524,446; 7,712,893; 6,491,907; 8,637,255; 7,186,522; 7,105,345; 6,759,237; 6,984,517; 6,962,815; 7,749,492; 7,259,151; and 6,156,303; U.S. Publication Nos. 2013/0295614; 2015/0065562; 2014/0364338; 2013/0323226; 2014/0359799; 2013/0059732; 2014/0037585; 2014/0056854; 2013/0296409; 2014/0335054 2013/0195801; 2012/0070899; 2011/0275529; 2011/0171262; 2009/0215879; 2010/0297177; 2010/0203083; 2009/0317417; 2009/0202490; 2012/0220492; 2006/0292117; and 2004/0002159; European Publication Numbers 2692731 AI; 2383346 BI; 2359865 BI; 2359866 BI; 2359867 BI; and 2357010 BI; 1791858 BI; 1668143 BI; 1660678 BI; 1664314 BI; 1496944 BI; 1456383 BI; 2341068 BI; 2338900 BI; 1456419 BI; 1310571 BI; 1456383 BI; 1633772 BI; and 1135468 BI; and PCT Publication Nos. WO 2014/124282; WO 2013/170078; WO 2014/160092; WO 2014/103957; WO 2014/052789; WO 2013/174760; WO 2013/123503; WO 2011/038187; WO 2008/124015; and WO 2003/054197).

For the purposes of the disclosure herein, AAV refers to the virus itself and derivatives thereof. Except where otherwise indicated, the terminology refers to all subtypes or serotypes and both replication-competent and recombinant forms. The term “AAV” includes, without limitation, AAV type 1 (AAV1), AAV type 2 (AAV2), AAV type 3A (AAV3A), AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV 10 or AAVrh10), avian AAV, bovine AAV, canine AAV, caprine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077.1 (AAV1), AF063497.1 (AAV1), NC_001401.2 (AAV2), AF043303.1 (AAV2), J01901.1 (AAV2), U48704.1 (AAV3A), NC_001729.1 (AAV3A), AF028705.1 (AAV3B), NC_0.001829.1 (AAV4), U89790.1 (AAV4), NC_006152.1 (AA5), AF085716.1 (AAV-5), AF028704.1 (AAV6), NC_006260.1 (AAV7), AF513851.1 (AAV7), AF513852.1 (AAV8) NC_006261.1 (AAV8), AY530579.1 (AAV9), AAT46337 (AAV10), and AA088208 (AAVrh10); the disclosures of which are incorporated by reference herein for teaching AAV nucleic acid and amino acid sequences. See also, e.g., Srivastava et al., J Virol. 45:555-564, 1983; Chiorini et al., J Virol 71:6823-6833, 1998; Chiorini et al., J Virol 73:1309-1319, 1999; Bantel-Schaal et al., J Virol 73:939-947, 1999; Xiao et al., J Virol 73:3994-4003, 1999; Muramatsu et al., Virology 221:208-217, 1996; Shade et al., J Virol 58:921-936, 1986; Gao et al., Proc Natl Acad Sci USA 99:11854-11859, 2002; PCT Publication Nos. WO 00/28061, WO 99/61601, and WO 98/11244; and U.S. Pat. No. 6,156,303.

Virus capsids may be mixed and matched with other vector components to form a hybrid pseudotype viral vector, for example the ITRs and capsid of the viral vector may come from different AAV serotypes. In one aspect, the ITRs can be from an AAV2 serotype while the capsid is from, for example, an AAV8, AAV9, AAV2, or AAV5 serotype. In addition, one of skill in the art will recognize that the vector capsid may also be a mosaic capsid (e.g., a capsid composed of a mixture of capsid proteins from different serotypes), or even a chimeric capsid (e.g., a capsid protein containing a foreign or unrelated protein sequence for generating markers and/or altering tissue tropism). It is contemplated that the viral vector of the invention may comprise an AAV8 capsid (e.g., SEQ ID NOs:24, 25, and 26, encoded by, for example, SEQ ID NO:23). It is also contemplated that the viral vector of the invention may comprise an AAV9 capsid (e.g., SEQ ID NOs:28, 29, and 30, encoded by, for example, SEQ ID NO:27). It is also contemplated that the viral vector of the invention may comprise an AAV2 capsid (e.g., SEQ ID NOs:32, 33, and 34, encoded by, for example, SEQ ID NO:31). It is further contemplated that the invention may comprise an AAV5 capsid (e.g., SEQ ID NOs:36, 37, and 38, encoded by, for example, SEQ ID NO:35).

In one aspect, the AAV is a self-complementary adeno-associated virus (scAAV).

In further particular aspects, the vector genome, e.g., single stranded vector genome, has a length greater than or about 4.1 kb and less than or about 4.9 kb, e.g., greater than or about 4.2 kb and less than or about 4.9 kb, greater than or about 4.3 kb and less than or about 4.9 kb, greater than or about 4.4 kb and less than or about 4.9 kb, greater than or about 4.5 kb and less than or about 4.9 kb, greater than or about 4.6 kb and less than or about 4.9 kb, greater than or about 4.7 kb and less than or about 4.9 kb, greater than or about 4.8 kb and less than or about 4.9 kb, greater than or about 4.1 kb and less than or about 4.8 kb, greater than or about 4.1 kb and less than or about 4.7 kb, greater than or about 4.1 kb and less than or about 4.6 kb, greater than or about 4.1 kb and less than or about 4.5 kb, greater than or about 4.1 kb and less than or about 4.4 kb, greater than or about 4.1 kb and less than or about 4.3 kb, greater than or about 4.1 kb and less than or about 4.2 kb, greater than or about 4.2 kb and less than or about 4.8 kb, greater than or about 4.3 kb and less than or about 4.7 kb, greater than or about 4.4 kb and less than or about 4.6 kb, about 4.1 kb, about 4.2 kb, about 4.3 kb, about 4.4 kb, about 4.5 kb, about 4.6 kb, about 4.7 kb, about 4.8 kb, or about 4.9 kb.

In certain aspects, the invention is related to a vector genome, e.g., vector genome, e.g., single stranded vector genome, comprising, in the 5′ to 3′ direction: (i) a promoter selected from ProA18 promoter and ProB4 promoter, and active fragments thereof; and (ii) a recombinant nucleotide sequence comprising a CYP4V2 (e.g., human CYP4V2) coding sequence.

In certain aspects, the invention is related to a vector genome, e.g., single stranded vector genome, comprising, in the 5′ to 3′ direction: (i) a 5′ ITR, (ii) a promoter, (iii) a recombinant nucleotide sequence comprising a CYP4V2 coding sequence, (iv) a polyadenylation (polyA) signal sequence, and (v) a 3′ ITR. In certain aspects of the invention, the vector genome, e.g., single stranded vector genome, comprises in the 5′ to 3′ direction: (i) a 5′ ITR, (ii) a promoter, (iii) an intron, (iv) a recombinant nucleotide sequence comprising a CYP4V2 coding sequence, (v) a polyA signal sequence, and (vi) a 3′ ITR. In some embodiments, the vector genome, e.g., single stranded vector genome, comprises in the 5′ to 3′ direction: (i) a 5′ ITR, (ii) a promoter, (iii) a recombinant nucleotide sequence comprising a CYP4V2 coding sequence, (iv) a regulatory element, (v) a polyA signal sequence, and (vi) a 3′ ITR. In certain aspects of the invention, the vector genome, e.g., single stranded vector genome, comprises in the 5′ to 3′ direction: (i) a 5′ ITR, (ii) a promoter, (iii) an intron, (iv) a recombinant nucleotide sequence comprising a CYP4V2 coding sequence, (v) a regulatory element, (vi) a polyA signal sequence, and (vii) a 3′ ITR. Elements of the vector can have sequences with greater than or about 80%, 85%, 90%, 91%, 92%, 93%, 94%, 95%, 96%, 97%, 98%, 99%, or 100% identity to the sequences described in Table 2.

TABLE 2
Nucleotide and amino acid sequences of viral vector elements

| ELEMENT | SEQUENCE IDENTIFIER (SEQ ID NO:) AND SEQUENCE |
|---|---|
| 5′ ITR | 1 |
| | CTGCGCGCTCGCTCGCTCACTGAGGCCGCCCGGGCAAAGCCCGG |
| | GCGTCGGGCGACCTTTGGTCGCCCGGCCTCAGTGAGCGAGCGAG |
| | CGCGCAGAGAGGGAGTGGCCAACTCCATCACTAGGGGTTCCT |
| ProA18 promoter | 2 |
| | ATTGAAGACCTCAGACTTTAGAGATACCAGAGCTATGGGATACC |
| | TGCTGAGAAAAGCTGCTAACAGGGAGTGGAACCAGACCAGGAAA |
| | AAGAAGTTTGTTACAGTCAACAAAGATGAATGGAATTGGAGATC |
| | TGATGAGCACTCTGACATTAGAAATGGAGATGCAGAGTTTGGAG |
| | TTTGCCTAGCTCTTTTTTGGGGTGTGGGGTGGGGTGGGTCCTTG |
| | TTTGCTCCAGTATTTCCTCACAATGACAATTTAGAATGATGGTG |
| | TATACACTGCGGTATTTGAGGTATGTGATCTGCTTTTTGATTTT |
| | GACTTTATAGGAGATTACAGATAAGTGATCAAATGAAACTCAGA |
| | AAAGACTTTGACCTTTAGACTTTTAACATTATTGAGAATGCCAT |
| | AGACTATGGAGACTTTTGAAGTGGGGACTAAATTTATTTTGCAT |
| | CATGCTTTGGCTAGGTATGGCCTCCATAGACTCATCTGTTCGAA |
| | CAAGCCTAAGGGAGCCAAGGGGTGGAATGTGGTGGTTTGAATAT |
| | GCTTGGCCCAGGAAGGACACTATTAGGAGGTATGGACTTGCTGG |
| | AAGAAGCTTGTCACTGTGGGAGTGGGCTTTGAGATCCTTTTTCT |
| | ACCTTGATGATAATGGACCAAACGTCTGAACCTGTAAACCAGTC |
| | CCAATTAAATGTTTTCTTTTATAAGAGTTGCCTTGGTCATGGTG |
| | TATACTCACAGCAATGTAAACTCTAAGATGGGGGACAATGGGAG |
| | GTGCCAGGGCCTACATGATAGAAGGACAGGCATTGTTACAATTC |
| | CACCTACCACTCACTACATGGTCTTGGACTGATTTGCAACCAAC |
| | TTCTCTCCAATGTCCTCCTCGGAAATAGAGATGTCCTGGCATCT |
| | CACCCATGGGGTTATATTTAGGAGGCTTTTGATGATCATCCACT |
| | AGTTCAGTCTAATATCTACTACTTTAATAGACATGAGTTCCTTT |
| | GCTAATAACCCTCTGGGATTTAGTTTCTCCATCTGAAAATTAGT |
| | TGTCCTGTGGCTCATTGTTTTCTCGTGAGAATTTCAGCATGAGC |
| | CAGTACAAAAGTTGTACCAAGACCTTGTGTGTAGAAGCCGAAGT |
| | TCTTAGTGGGTCATGAGGTACTTTCAAAAAGAATGCAAGCCATT |
| | CTTTATCCTGAGAGATATTTTATGATTGCATAGCTCAATGGCTG |
| | TCTGTGAGACAGGAAGTGAAGCCCTAAATCCATGATGGAGTTCA |
| | ACCAGCACTTAACTAGGGAAGGGCATGAAGCAGAAATGACTCAG |
| | TTGACAGGAAAACCATCCAATGGCAGCAGTGCAGAGCAGACAGC |
| | CAGTCATGGCAGACTCAGTACCAGAGGTCAAGGGTCAGGTACTA |
| | GTCAACATTTGCTTTATGACAGCACGTAACTTTACAAACCTCAC |
| | CCTGCCCACCAAATGCTTGCCTACACATACTTCTGAGCCTGTGA |
| | ATGAACATACAACACACACCCACACACATGCAAATGCACGCGCA |
| | CACACACACACACACTCACACACACACACACACACACAGAGAGA |
| | GAGAGAGAGAGAGAGAGAGAGAGAGAGAGAGAGAGAGAGATGCA |
| | CACACACAGGCAGCTTGTCAGCAGCTGTGCACATACAAGGATCT |
| | GGCTATCCAATTCTCGGGGACAGCTGCAGCTCAAATCCTTTCTT |
| | CCACTTCCCTCCCTTAGTTATGCAACCCTTACCCAATTCAGCTT |
| | TCACTCACACACCATTTGGATCCAAGACCTTAATCCTGCCTAGT |
| | GGGCTGGAATGAACAAGACAGAGCTCATTCCAGCTTCACAAAAG |
| | CTGCACTATCCATCTACTGAATGGATTCTTTCTATGTGAGCCAA |
| | GAGGAAGACTTAGAAGGATAAGAAATAAAAAAGGTGTTATTAGT |
| | CTACCATAATAATCTCCACATGCCAGCAAGGGAGTGACCATTTA |
| | AAAGGAGAGACCTAGCTTCAGAGAGCCAGAAAAGAGCTGTGTAG |
| | CTGACAGAGGGAGTCCAGGG |
| ProB4 promoter | 3 |
| | GTTCTTTGGGTTCCATATTTTCCTCATTCCCCTTCAAGTCTAGC |
| | TGGATTGCAAAGTTAAGCTTATGATTATGAATAAAAACTATTGA |
| | GTATATTTTCAAGGAAATAGTGACTTACCTTTTAAGGTCACTTC |
| | CTGCTCTTTCATCTGACCCAGAAACACTGAATAATAAAGCCATC |
| | ATTTGTTGAATGACCACTGTGTAATTTTTATTTAGAACTCTAAT |
| | GTTGGTAATCTGCTTGGGGCTGTGGGTTAAGAATCTTTGGGTTG |
| | CTTGAGGAAATGCACTTAATTAAAATAAACATTAAACTTTTCCA |
| | CACTGGCCTTTGTCTCCCCCTGGCGGATCCTAGCAACAGATACC |
| | AGCAAGTTGTCTTACTTGGTATTTAGGGCAATGTGATGCAGCCC |
| | TAACCCAAAGTGTTGTCACACCAAACCCTTCACACCCAAGAGGA |
| | TGTTTCGCTCTGAAGTCATCAGCTCTAACAAATATTTGTAAGTA |
| | AACAGGTTTTTCTTTAAGGAGGCCTTCATTTGTAACCAACACCA |
| | TGCTCTCCTTTTTCGCTCTCTGAAGTCATCAGCTCTAAAGGAAA |
| | TCAATCTCAATGCCCTATTATACCCTGATCTCTGGCCTTTGGAG |
| | GGGCTGTTCAAAGATTCTCTGAGGACTTATAGGCTAATAAGGTT |
| | GAGGATTTTGTGATTTAAAGAACTCCTTAATCCACTGCTTCCTG |
| | CCTACCATAAGCCAAGGCCACCCTTGCCTTTTTTTTTCTTATCC |
| | CCTGCTGAACCTGAAAAACCCTCTCTTTTCTACAGTTTTCTGTT |
| | CCCAGGCCCCAAGTACAGTTAAACAATTCAACTCAACTTGTTTA |
| | ATCCTCTCGTCTTAAATTATTCACATTGCCAAGTATTTCAACAG |
| | AATGTACTGGCAAGTTCATAAACAGAGTGCAAATCACCCAAGCA |
| | GAAGTAATTTACTGCTTTTAAGTCAAAGCCCTGCTTAGCTAGGG |
| | AGTCAGCTGTAGGAATTAACCAGAACAAAATCCTGATTAAACAG |
| | CTAATCGGTTCTACTCCGCTAACCCAGTGGCCTGAGCAAATCAC |
| | AAGGAGGATTGGTTCCTTGCATCTGGTTTGGGCACCGCCCACCT |
| | GGAACCCTAGGGGCGGGGCCGCTTAAGTGGCAGGAGGGATATAT |
| | ATCAGGATGCAAAAGACCCTGCCAACTGGAAAAATAAGAAGGCA |
| | GCAGCTTTGGATTTTCCACTCATGGAAGAGGGGAGGAAAGGGCA |
| | GAGGGTGGGGGCTTCCTTGGGAAGCTTGGGATGAGTCACCACGG |
| | GATTGGGGAGTGGAGGGTGGATAGGATATGGAGAAGGGTGG |
| Human growth | 9 |
| hormone (hGH) | TTCGAACAGGTAAGCGCCCCTAAAATCCCTTTGGGCACAATGTG |
| intron | TCCTGAGGGGAGAGGCAGCGACCTGTAGATGGGACGGGGGCACT |
| | AACCCTCAGGTTTGGGGCTTCTGAATGTGAGTATCGCCATGTAA |
| | GCCCAGTATTTGGCCAATCTCAGAAAGCTCCTGGTCCCTGGAGG |
| | GATGGAGAGAGAAAAACAAACAGCTCCTGGAGCAGGGAGAGTGC |
| | TGGCCTCTTGCTCTCCGGCTCCCTCTGTTGCCCTCTGGTTTCTC |
| | CCCAGGTT |
| Simian Virus 40 | 10 |
| (SV40) intron | AACTGAAAAACCAGAAAGTTAACTGGTAAGTTTAGTCTTTTTGT |
| | CTTTTATTTCAGGTCCCGGATCCGGTGGTGGTGCAAATCAAAGA |
| | ACTGCTCCTCAGTGGATGTTGCCTTTACTTCTAGGCCTGTACGG |
| | AAGTGTTACTTCTGCTCTAAAAGCTGCGGAATTGTACCCGCCCC |
| | GGGATCC |
| Human beta | 11 |
| gobin intron | CGAATCCCGGCCGGGAACGGTGCATTGGAACGCGGATTCCCCGT |
| | GCCAAGAGTGACGTAAGTACCGCCTATAGAGTCTATAGGCCCAC |
| | AAAAAATGCTTTCTTCTTTTAATATACTTTTTTGTTTATCTTAT |
| | TTCTAATACTTTCCCTAATCTCTTTCTTTCAGGGCAATAATGAT |
| | ACAATGTATCATGCCTCTTTGCACCATTCTAAAGAATAACAGTG |
| | ATAATTTCTGGGTTAAGGCAATAGCAATATTTCTGCATATAAAT |
| | ATTTCTGCATATAAATTGTAACTGATGTAAGAGGTTTCATATTG |
| | CTAATAGCAGCTACAATCCAGCTACCATTCTGCTTTTATTTTAT |
| | GGTTGGGATAAGGCTGGATTATTCTGAGTCCAAGCTAGGCCCTT |
| | TTGCTAATCATGTTCATACCTCTTATCTTCCTCCCACAGCTCCT |
| | GGGCAACGTGCTGGTCTGTGTGCTGGCCCATCACTTTGGCAAAG |
| | AATTGGGAT |
| Kozak sequence | 12 |
| | GCCACC |
| Homo sapiens | 13 |
| CYP4V2 | ATGGCGGGGCTCTGGCTGGGGCTCGTGTGGCAGAAGCTGCTGCT |
| Wild-type CDS | GTGGGGCGCGGCGAGTGCCCTTTCCCTGGCCGGCGCCAGTCTGG |
| NM_207352.4 | TCCTGAGCCTGCTGCAGAGGGTGGCGAGCTACGCGCGGAAATGG |
| | CAGCAGATGCGGCCCATCCCCACGGTGGCCCGCGCCTACCCACT |
| | GGTGGGCCACGCGCTGCTGATGAAGCCGGACGGGCGAGAATTTT |
| | TTCAGCAGATCATTGAGTACACAGAGGAATACCGCCACATGCCG |
| | CTGCTGAAGCTCTGGGTCGGGCCAGTGCCCATGGTGGCCCTTTA |
| | TAATGCAGAAAATGTGGAGGTAATTTTAACTAGTTCAAAGCAAA |
| | TTGACAAATCCTCTATGTACAAGTTTTTAGAACCATGGCTTGGC |
| | CTAGGACTTCTTACAAGTACTGGAAACAAATGGCGCTCCAGGAG |
| | AAAGATGTTAACACCCACTTTCCATTTTACCATTCTGGAAGATT |
| | TCTTAGATATCATGAATGAACAAGCAAATATATTGGTTAAGAAA |
| | CTTGAAAAACACATTAACCAAGAAGCATTTAACTGCTTTTTTTA |
| | CATCACTCTTTGTGCCTTAGATATCATCTGTGAAACAGCTATGG |
| | GGAAGAATATTGGTGCTCAAAGTAATGATGATTCCGAGTATGTC |
| | CGTGCAGTTTATAGAATGAGTGAGATGATATTTCGAAGAATAAA |
| | GATGCCCTGGCTTTGGCTTGATCTCTGGTACCTTATGTTTAAAG |
| | AAGGATGGGAACACAAAAAGAGCCTTCAGATCCTACATACTTTT |
| | ACCAACAGTGTCATCGCTGAACGGGCCAATGAAATGAACGCCAA |
| | TGAAGACTGTAGAGGTGATGGCAGGGGCTCTGCCCCCTCCAAAA |
| | ATAAACGCAGGGCCTTTCTTGACTTGCTTTTAAGTGTGACTGAT |
| | GACGAAGGGAACAGGCTAAGTCATGAAGATATTCGAGAAGAAGT |
| | TGACACCTTCATGTTTGAGGGGCACGATACAACTGCAGCTGCAA |
| | TAAACTGGTCCTTATACCTGTTGGGTTCTAACCCAGAAGTCCAG |
| | AAAAAAGTGGATCATGAATTGGATGACGTGTTTGGGAAGTCTGA |
| | CCGTCCCGCTACAGTAGAAGACCTGAAGAAACTTCGGTATCTGG |
| | AATGTGTTATTAAGGAGACCCTTCGCCTTTTTCCTTCTGTTCCT |
| | TTATTTGCCCGTAGTGTTAGTGAAGATTGTGAAGTGGCAGGTTA |
| | CAGAGTTCTAAAAGGCACTGAAGCCGTCATCATTCCCTATGCAT |
| | TGCACAGAGATCCGAGATACTTCCCCAACCCCGAGGAGTTCCAG |
| | CCTGAGCGGTTCTTCCCCGAGAATGCACAAGGGCGCCATCCATA |
| | TGCCTACGTGCCCTTCTCTGCTGGCCCCAGGAACTGTATAGGTC |
| | AAAAGTTTGCTGTGATGGAAGAAAAGACCATTCTTTCGTGCATC |
| | CTGAGGCACTTTTGGATAGAATCCAACCAGAAAAGAGAAGAGCT |
| | TGGTCTAGAAGGACAGTTGATTCTTCGTCCAAGTAATGGCATCT |
| | GGATCAAGTTGAAGAGGAGAAATGCAGATGAACGCTAA |
| Codon- | 14 |
| optimized | ATGGCCGGACTGTGGCTGGGACTGGTCTGGCAGAAGTTATTACT |
| CYP4V2 | GTGGGGAGCTGCCTCCGCTCTGTCTTTAGCTGGAGCTTCTTTAG |
| sequence | TGCTGTCTTTACTGCAGAGGGTCGCCTCCTATGCTAGGAAGTGG |
| | CAGCAGATGAGGCCTATTCCTACCGTGGCCAGAGCCTATCCTTT |
| | AGTGGGCCACGCTCTGCTGATGAAACCCGACGGAAGGGAGTTCT |
| | TCCAGCAGATCATCGAGTACACCGAAGAGTACAGACACATGCCT |
| | TTACTGAAACTGTGGGTGGGACCCGTTCCTATGGTGGCTTTATA |
| | CAATGCCGAGAATGTGGAGGTGATTTTAACCAGCAGCAAGCAGA |
| | TCGACAAGTCCAGCATGTATAAGTTTTTAGAGCCTTGGCTCGGT |
| | TTAGGACTGCTGACCTCCACTGGTAATAAGTGGAGGTCTCGTAG |
| | GAAAATGCTGACCCCCACCTTTCACTTCACCATTTTAGAGGACT |
| | TTTTAGATATCATGAACGAGCAAGCTAACATTTTAGTGAAGAAG |
| | CTCGAAAAGCACATTAACCAAGAAGCTTTCAACTGTTTTTTCTA |
| | CATCACTTTATGCGCCCTCGATATCATCTGCGAGACAGCCATGG |
| | GCAAGAACATTGGCGCTCAGAGCAACGACGATTCCGAGTACGTG |
| | AGGGCTGTCTACAGAATGAGCGAGATGATCTTCAGAAGAATCAA |
| | GATGCCTTGGCTGTGGCTGGACCTCTGGTATTTAATGTTTAAGG |
| | AAGGCTGGGAGCATAAGAAGTCTTTACAGATTTTACATACATTT |
| | ACCAACAGCGTGATCGCCGAGAGGGCCAATGAAATGAACGCCAA |
| | CGAGGATTGTCGTGGCGACGGAAGAGGCTCCGCTCCTTCCAAGA |
| | ACAAGAGGAGAGCCTTTTTAGATCTCTTATTATCCGTGACAGAC |
| | GATGAGGGCAATAGGCTGAGCCACGAGGACATCAGAGAAGAGGT |
| | GGACACCTTCATGTTCGAGGGACACGACACAACCGCCGCCGCCA |
| | TCAATTGGTCTTTATATTTACTCGGCAGCAACCCCGAGGTGCAA |
| | AAAAAGGTCGACCACGAGCTCGACGACGTGTTCGGCAAGAGCGA |
| | TCGTCCCGCCACAGTGGAAGATTTAAAGAAGCTGAGGTATCTCG |
| | AGTGCGTGATCAAAGAGACTTTAAGACTGTTCCCCAGCGTGCCT |
| | CTGTTTGCTCGTTCCGTGTCCGAAGACTGCGAGGTGGCTGGATA |
| | TCGTGTCCTCAAGGGCACCGAGGCCGTGATCATTCCCTACGCCC |
| | TCCATCGTGATCCCAGATACTTCCCCAATCCCGAGGAGTTCCAG |
| | CCCGAAAGGTTCTTCCCCGAAAACGCTCAAGGCAGACACCCTTA |
| | CGCTTACGTGCCTTTCTCCGCCGGCCCTCGTAACTGCATTGGCC |
| | AGAAATTCGCCGTCATGGAGGAAAAGACCATTTTATCTTGTATT |
| | TTAAGGCACTTCTGGATCGAAAGCAATCAGAAAAGGGAGGAACT |
| | CGGTTTAGAAGGACAGCTGATTTTAAGACCCAGCAACGGCATTT |
| | GGATCAAGCTGAAGAGGAGGAACGCCGACGAGAGGTGA |
| Homo sapiens | 15 |
| CYP4V2 Gene | MAGLWLGLVWQKLLLWGAASALSLAGASLVLSLLQRVASYARKW |
| Product | QQMRPIPTVARAYPLVGHALLMKPDGREFFQQIIEYTEEYRHMP |
| NP_997235.3 | |
| | LLKLWVGPVPMVALYNAENVEVILTSSKQIDKSSMYKFLEPWLG |
| | LGLLTSTGNKWRSRRKMLTPTFHFTILEDFLDIMNEQANILVKK |
| | LEKHINQEAFNCFFYITLCALDIICETAMGKNIGAQSNDDSEYV |
| | RAVYRMSEMIFRRIKMPWLWLDLWYLMFKEGWEHKKSLQILHTF |
| | TNSVIAERANEMNANEDCRGDGRGSAPSKNKRRAFLDLLLSVTD |
| | DEGNRLSHEDIREEVDTFMFEGHDTTAAAINWSLYLLGSNPEVQ |
| | KKVDHELDDVFGKSDRPATVEDLKKLRYLECVIKETLRLFPSVP |
| | LFARSVSEDCEVAGYRVLKGTEAVIIPYALHRDPRYFPNPEEFQ |
| | PERFFPENAQGRHPYAYVPFSAGPRNCIGQKFAVMEEKTILSCI |
| | LRHFWIESNQKREELGLEGQLILRPSNGIWIKLKRRNADER |
| Hepatitis B | 16 |
| virus | TAAACAGGCCTATTGATTGGAAAGTATGTCAACGAATTGTGGGT |
| regulatory | CTTTTGGGGTTTGCTGCCCCTTTTACGCAATGTGGATATCCTGC |
| element (HPRE) | TTTAATGCCTTTATATGCATGTATACAAGCAAAACAGGCTTTTA |
| | CTTTCTCGCCAACTTACAAGGCCTTTCTAAGTAAACAGTATCTG |
| | ACCCTTTACCCCGTTGCTCGGCAACGGCCTGGTCTGTGCCAAGT |
| | GTTTGCTGACGCAACCCCCACTGGTTGGGGCTTGGCCATAGGCC |
| | ATCAGCGCATGCGTGGAACCTTTGTGTCTCCTCTGCCGATCCAT |
| | ACTGCGGAACTCCTAGCCGCTTGTTTTGCTCGCAGCAGGTCTGG |
| | AGCGAAACTCATCGGGACTGACAATTCTGTCGTGCTCTCCCGCA |
| | AGTATACATCGTTTCCAGGGCTGCTAGGCTGTGCTGCCAACTGG |
| | ATCCTGCGCGGGACGTCCTTTGTTTACGTCCCGTCGGCGCTGAA |
| | TCCCGCGGACGACCCCTCCCGGGGCCGCTTGGGGCTCTACCGCC |
| | CGCTTCTCCGTCTGCCGTACCGACCGACCACGGGGCGCACCTCT |
| | CTTTACGCGGACTCCCCGTCTGTGCCTTCTCATCTGCCGGACCG |
| | TGTGCACTTCGCTTCACCTCTGCACGTCGCATGGAGACCACCGT |
| | GAACGCCCACCGGAACCTGCCCAAGGTCTTGCATAAGAGGACTC |
| | TTGGACTTTCAGCAATGTCAACTCGA |
| Woodchuck | 17 |
| hepatitis virus | TCAACCTCTGGATTACAAAATTTGTGAAAGATTGACTGGTATTC |
| regulatory | TTAACTATGTTGCTCCTTTTACGCTATGTGGATACGCTGCTTTA |
| element (WPRE) | ATGCCTTTGTATCATGCTATTGCTTCCCGTATGGCTTTCATTTT |
| | CTCCTCCTTGTATAAATCCTGGTTGCTGTCTCTTTATGAGGAGT |
| | TGTGGCCCGTTGTCAGGCAACGTGGCGTGGTGTGCACTGTGTTT |
| | GCTGACGCAACCCCCACTGGTTGGGGCATTGCCACCACCTGTCA |
| | GCTCCTTTCCGGGACTTTCGCTTTCCCCCTCCCTATTGCCACGG |
| | CGGAACTCATCGCCGCCTGCCTTGCCCGCTGCTGGACAGGGGCT |
| | CGGCTGTTGGGCACTGACAATTCCGTGGTGTTGTCGGGGAAATC |
| | ATCGTCCTTTCCTTGGCTGCTCGCCTGTGTTGCCACCTGGATTC |
| | TGCGCGGGACGTCCTTCTGCTACGTCCCTTCGGCCCTCAATCCA |
| | GCGGACCTTCCTTCCCGCGGCCTGCTGCCGGCTCTGCGGCCTCT |
| | TCCGCGTCTTCGCCTTCGCCCTCAGACGAGTCGGATCTCCCTTT |
| | GGGCCGCCTCCCCGCA |
| Bovine Growth | 18 |
| Hormone (bGH) | GATCTGGATGATGACGACAAGTGAGGATCCCTGTGCCTTCTAGT |
| polyA signal | TGCCAGCCATCTGTTGTTTGCCCCTCCCCCGTGCCTTCCTTGAC |
| sequence | CCTGGAAGGTGCCACTCCCACTGTCCTTTCCTAATAAAATGAGG |
| | AAATTGCATCGCATTGTCTGAGTAGGTGTCATTCTATTCTGGGG |
| | GGTGGGGTGGGGCAGGACAGCAAGGGGGAGGATTGGGAAGAGAA |
| | TAGCAGGCATGCTGGGGAGAATTCA |
| Simian Virus 40 | 19 |
| (5V40) polyA | GATCATAATCAGCCATACCACATTTGTAGAGGTTTTACTTGCTT |
| signal sequence | TAAAAAACCTCCCACACCTCCCCCTGAACCTGAAACATAAAATG |
| | AATGCAATTGTTGTTGTTAACTTGTTTATTGCAGCTTATAATGG |
| | TTACAAATAAAGCAATAGCATCACAAATTTCACAAATAAAGCAT |
| | TTTTTTCACTGCATTCTAGTTGTGGTTTGTCCAAACTCATCAAT |
| | GTATCTTATCATGTCT |
| SYNUCLEIN | 20 |
| INTRONIC | GGGCCCCGGTGTTATCTCATTCTTTTTTCTCCTCTGTAAGTTGA |
| SEQUENCE AS | CATGTGATGTGGGAACAAAGGGGATAAAGTCATTATTTTGTGCT |
| STUFFER | AAAATCGTAATTGGAGAGGACCTCCTGTTAGCTGGGCTTTCTTC |
| SEQUENCE | TATTTATTGTGGTGGTTACTGGAGTTCCTTCTTCTAGTTTTAGG |
| | ATATATATATATATTTTTTTTTTTTCTTTCCCTGAAGATATAAT |
| | AATATATATACTTCTGAAGATTGAGATTTTTAAATTAGTTGTAT |
| | TGAAAACTAGCTAATCAGCAATTTAAGGCTAGCTTGAGACTTAT |
| | GTCTTGAATTTGTTTTTGTAGGCTCCAAAACCAAGGAGGGAGTG |
| | GTGCATGGTGTGGCAACAGGTAAGCTCCATTGTGCTTATATCCA |
| | AAGATGATATTTAAAGTATCTAGTGATTAGTGTGGCCCAGTATT |
| | CAAGATTCCTATGAAATTGTAAAACAATCACTGAGCATTCTAAG |
| | AACATATCAGTCTTATTGAAACTGAATTCTTTATAAAGTATTTT |
| | TAAAAAGGTAAATATTGATTATAAATAAAAAATATACTTGCCAA |
| | GAATAATGAGGGCTTTGAATTGATAAGCTATGTTTAATTTATAG |
| | TAAGTGGGCATTTAAATATTCTGACCAAAAATGTATTGACAAAC |
| | TGCTGACAAAAATAAAATGTGAATATTGCCATAATTTTAAAAAA |
| | AGAGTAAAATTTCTGTTGATTACAGTAAAATATTTTGACCTTAA |
| | ATTATGTTGATTACAATATTCCTTTGATAATTCAGAGTGCATTT |
| | CAGGAAACACCCTTGGACAGTCAGTAAATTGTTTATTGTATTTA |
| | TCTTTGTATTGTTATGGTATAGCTATTTGTACAAATATTATTGT |
| | GCAATTATTACATTTCTGATTATATTATTCATTTGGCCTAAATT |
| | TACCAAGAATTTGAACAAGTCAATTAGGTTTACAATCAAGAAAT |
| | ATCAAAAATGATGAAAAGGATGATAATCATCATCAGATGTTGAG |
| | GAAGATGACGATGAGAGTGCCAGAAATAGAGAAATCAAAGGAGA |
| | ACCAAAATTTAACAAATTAAAAGCCCACAGACTTGCTGTAATTA |
| | AGTTTTCTGTTGTAAGTACTCCACGTTTCCTGGCAGATGTGGTG |
| | AAGCAAAAGATATAATCAGAAATATAATTTATATGATCGGAAAG |
| | CATTAAACACAATAGTGCCTATACAAATAAAATGTTCCTATCAC |
| | TGACTTCTAAAATGGAAATGAGGACAATGATATGGGAATCTTAA |
| | TACAGTGTTGTGGATAGGACTAAAAACACAGGAGTCAGATCTTC |
| | TTGGTTCAACTTCCTGCTTACTCCTTACCAGCTGTGTGTTTTTT |
| | GCAAGGTTCTTCACCTCTATGTGATTTAGCTTCCTCATCTATAA |
| | AATAATTCAGTGAATTAATGTACACAAAACATCTGGAAAACAAA |
| | AGCAAACAATATGTATTTTATAAGTGTTACTTATAGTTTTATAG |
| | TGAACTTTCTTGTGCAACATTTTTACAACTAGTGGAGAAAAATA |
| | TTTCTTTAAATGAATACTTTTGATTTAAAAATCAGAGTGTAAAA |
| | ATAAAACAGACTCCTTTGAAACTAGTTCTGTTAGAAGTTAATTG |
| | TGCACCTTTAATGGGCTCTGTTGCAATCCAACAGAGAAGTAGTT |
| | AAGTAAGTGGACTATGATGGCTTCTAGGGACCTCCTATAAATAT |
| | GATATTGTGAAGCATGATTATAATAAGAACTAGATAACAGACAG |
| | GTGGAGACTCCACTATCTGAAGAGGGTCAACCTAGATGAATGGT |
| | GTTCCATTTAGTAGTTGAGGAAGAACCCATGAGGTTTAGAAAGC |
| | AGACAAGCATGTGGCAAGTTCTGGAGTCAGTGGTAAAAATTAAA |
| | GAACCCAACTATTACTGTCACCTAATGATCTAATGGAGACTGTG |
| | GAGATGGGCTGCATTTTTTTAATCTTCTCCAGAATGCCAAAATG |
| | TAAACACATATCTGTGTGTGTGTGTGTGTGTGTGTGTGTGTGTG |
| | AGAGAGAGAGAGAGAGAGAGAGAGACTGAAGTTTGTACAATTAG |
| | ACATTTTATAAAATGTTTTCTGAAGGACAGTGGCTCACAATCTT |
| | AAGTTTCTAACATTGTACAATGTTGGGAGACTTTGTATACTTTA |
| | TTTTCTCTTTAGCATATTAAGGAATCTGAGATGTCCTACAGTAA |
| | AGAAATTTGCATTACATAGTTAAAATCAGGGTTATTCAAACTTT |
| | TTGATTATTGAAACCTTTCTTCATTAGTTACTAGGGTTGAATGA |
| | AACTAGTGTTCCACAGAAAACTATGGGAAATGTTGCTAGGCAGT |
| | AAGGACATGGTGATTTCAGCATGTGCAATATTTACAGCGATTGC |
| | ACCCATGGACCACCCTGGCAGTAGTGAAATAACCAAAAATGCTG |
| | TCATAACTAGTATGGCTATGAGAAACACATTGGG |
| RLBP1 INTRONIC | 21 |
| SEQUENCE AS | ATTCTCCAGGTTGAGCCAGACCAATTTGATGGTAGATTTAGCAA |
| STUFFER | ATAAAAATACAGGACACCCAGTTAAATGTGAATTTCCGATGAAC |
| SEQUENCE | AGCAAATACTTTTTTAGTATTAAAAAAGTTCACATTTAGGCTCA |
| | CGCCTGTAATCCCAGCACTTTGGGAGGCCGAGGCAGGCAGATCA |
| | CCTGAGGTCAGGAGTTCGAGACCAGCCTGGCCAACATGGTGAAA |
| | CCCCATCTCCACTAAAAATACCAAAAATTAGCCAGGCGTGCTGG |
| | TGGGCACCTGTAGTTCCAGCTACTCAGGAGGCTAAGGCAGGAGA |
| | ATTGCTTGAACCTGGGAGGCAGAGGTTGCAGTGAGCTGAGATCG |
| | CACCATTGCACTCTAGCCTGGGCGACAAGAACAAAACTCCATCT |
| | CAAAAAAAAAAAAAAAAAAAAAGTTCACATTTAACTGGGCATTC |
| | TGTATTTAATTGGTAATCTGAGATGGCAGGGAACAGCATCAGCA |
| | TGGTGTGAGGGATAGGCATTTTTTCATTGTGTACAGCTTGTAAA |
| | TCAGTATTTTTAAAACTCAAAGTTAATGGCTTGGGCATATTTAG |
| | AAAAGAGTTGCCGCACGGACTTGAACCCTGTATTCCTAAAATCT |
| | AGGATCTTGTTCTGATGGTCTGCACAACTGGCTGGGGGTGTCCA |
| | GCCACTGTCCCTCTTGCCTGGGCTCCCCAGGGCAGTTCTGTCAG |
| | CCTCTCCATTTCCATTCCTGTTCCAGCAAAACCCAACTGATAGC |
| | ACAGCAGCATTTCAGCCTGTCTACCTCTGTGCCCACATACCTGG |
| | ATGTCTACCAGCCAGAAAGGTGGCTTAGATTTGGTTCCTGTGGG |
| | TGGATTATGGCCCCCAGAACTTCCCTGTGCTTGCTGGGGGTGTG |
| | GAGTGGAAAGAGCAGGAAATGGGGGACCCTCCGATACTCTATGG |
| | GGGTCCTCCAAGTCTCTTTGTGCAAGTTAGGGTAATAATCAATA |
| | TGGAGCTAAGAAAGAGAAGGGGAACTATGCTTTAGAACAGGACA |
| | CTGTGCCAGGAGCATTGCAGAAATTATATGGTTTTCACGACAGT |
| | TCTTTTTGGTAGGTACTGTTATTATCCTCAGTTTGCAGATGAGG |
| | AAACTGAGACCCAGAAAGGTTAAATAACTTGCTAGGGTCACACA |
| | AGTCATAACTGACAAAGCCTGATTCAAACCCAGGTCTCCCTAAC |
| | CTTTAAGGTTTCTATGACGCCAGCTCTCCTAGGGAGTTTGTCTT |
| | CAGATGTCTTGGCTCTAGGTGTCAAAAAAAGACTTGGTGTCAGG |
| | CAGGCATAGGTTCAAGTCCCAACTCTGTCACTTACCAACTGTGA |
| | CTAGGTGATTGAACTGACCATGGAACCTGGTCACATGCAGGAGC |
| | AGGATGGTGAAGGGTTCTTGAAGGCACTTAGGCAGGACATTTAG |
| | GCAGGAGAGAAAACCTGGAAACAGAAGAGCTGTCTCCAAAAATA |
| | CCCACTGGGGAAGCAGGTTGTCATGTGGGCCATGAATGGGACCT |
| | GTTCTGG |
| 3′ ITR | 22 |
| | AGGAACCCCTAGTGATGGAGTTGGCCACTCCCTCTCTGCGCGCT |
| | CGCTCGCTCACTGAGGCCGGGCGACCAAAGGTCGCCCGACGCCC |
| | GGGCTTTGCCCGGGCGGCCTCAGTGAGCGAGCGAGCGCGCAG |
| AAV8 Capsid | 23 |
| Coding Sequence | ATGGCTGCCGATGGTTATCTTCCAGATTGGCTCGAGGACAACCT |
| | CTCTGAGGGCATTCGCGAGTGGTGGGCGCTGAAACCTGGAGCCC |
| | CGAAGCCCAAAGCCAACCAGCAAAAGCAGGACGACGGCCGGGGT |
| | CTGGTGCTTCCTGGCTACAAGTACCTCGGACCCTTCAACGGACT |
| | CGACAAGGGGGAGCCCGTCAACGCGGCGGACGCAGCGGCCCTCG |
| | AGCACGACAAGGCCTACGACCAGCAGCTGCAGGCGGGTGACAAT |
| | CCGTACCTGCGGTATAACCACGCCGACGCCGAGTTTCAGGAGCG |
| | TCTGCAAGAAGATACGTCTTTTGGGGGCAACCTCGGGCGAGCAG |
| | TCTTCCAGGCCAAGAAGCGGGTTCTCGAACCTCTCGGTCTGGTT |
| | GAGGAAGGCGCTAAGACGGCTCCTGGAAAGAAGAGACCGGTAGA |
| | GCCATCACCCCAGCGTTCTCCAGACTCCTCTACGGGCATCGGCA |
| | AGAAAGGCCAACAGCCCGCCAGAAAAAGACTCAATTTTGGTCAG |
| | ACTGGCGACTCAGAGTCAGTTCCAGACCCTCAACCTCTCGGAGA |
| | ACCTCCAGCAGCGCCCTCTGGTGTGGGACCTAATACAATGGCTG |
| | CAGGCGGTGGCGCACCAATGGCAGACAATAACGAAGGCGCCGAC |
| | GGAGTGGGTAGTTCCTCGGGAAATTGGCATTGCGATTCCACATG |
| | GCTGGGCGACAGAGTCATCACCACCAGCACCCGAACCTGGGCCC |
| | TGCCCACCTACAACAACCACCTCTACAAGCAAATCTCCAACGGG |
| | ACATCGGGAGGAGCCACCAACGACAACACCTACTTCGGCTACAG |
| | CACCCCCTGGGGGTATTTTGACTTTAACAGATTCCACTGCCACT |
| | TTTCACCACGTGACTGGCAGCGACTCATCAACAACAACTGGGGA |
| | TTCCGGCCCAAGAGACTCAGCTTCAAGCTCTTCAACATCCAGGT |
| | CAAGGAGGTCACGCAGAATGAAGGCACCAAGACCATCGCCAATA |
| | ACCTCACCAGCACCATCCAGGTGTTTACGGACTCGGAGTACCAG |
| | CTGCCGTACGTTCTCGGCTCTGCCCACCAGGGCTGCCTGCCTCC |
| | GTTCCCGGCGGACGTGTTCATGATTCCCCAGTACGGCTACCTAA |
| | CACTCAACAACGGTAGTCAGGCCGTGGGACGCTCCTCCTTCTAC |
| | TGCCTGGAATACTTTCCTTCGCAGATGCTGAGAACCGGCAACAA |
| | CTTCCAGTTTACTTACACCTTCGAGGACGTGCCTTTCCACAGCA |
| | GCTACGCCCACAGCCAGAGCTTGGACCGGCTGATGAATCCTCTG |
| | ATTGACCAGTACCTGTACTACTTGTCTCGGACTCAAACAACAGG |
| | AGGCACGGCAAATACGCAGACTCTGGGCTTCAGCCAAGGTGGGC |
| | CTAATACAATGGCCAATCAGGCAAAGAACTGGCTGCCAGGACCC |
| | TGTTACCGCCAACAACGCGTCTCAACGACAACCGGGCAAAACAA |
| | CAATAGCAACTTTGCCTGGACTGCTGGGACCAAATACCATCTGA |
| | ATGGAAGAAATTCATTGGCTAATCCTGGCATCGCTATGGCAACA |
| | CACAAAGACGACGAGGAGCGTTTTTTTCCCAGTAACGGGATCCT |
| | GATTTTTGGCAAACAAAATGCTGCCAGAGACAATGCGGATTACA |
| | GCGATGTCATGCTCACCAGCGAGGAAGAAATCAAAACCACTAAC |
| | CCTGTGGCTACAGAGGAATACGGTATCGTGGCAGATAACTTGCA |
| | GCAGCAAAACACGGCTCCTCAAATTGGAACTGTCAACAGCCAGG |
| | GGGCCTTACCCGGTATGGTCTGGCAGAACCGGGACGTGTACCTG |
| | CAGGGTCCCATCTGGGCCAAGATTCCTCACACGGACGGCAACTT |
| | CCACCCGTCTCCGCTGATGGGCGGCTTTGGCCTGAAACATCCTC |
| | CGCCTCAGATCCTGATCAAGAACACGCCTGTACCTGCGGATCCT |
| | CCGACCACCTTCAACCAGTCAAAGCTGAACTCTTTCATCACGCA |
| | ATACAGCACCGGACAGGTCAGCGTGGAAATTGAATGGGAGCTGC |
| | AGAAGGAAAACAGCAAGCGCTGGAACCCCGAGATCCAGTACACC |
| | TCCAACTACTACAAATCTACAAGTGTGGACTTTGCTGTTAATAC |
| | AGAAGGCGTGTACTCTGAACCCCGCCCCATTGGCACCCGTTACC |
| | TCACCCGTAATCTGTAA |
| AAV8 Capsid | 24 |
| Sequence (VP1) | MAADGYLPDWLEDNLSEGIREWWALKPGAPKPKANQQKQDDGRG |
| | LVLPGYKYLGPFNGLDKGEPVNAADAAALEHDKAYDQQLQAGDN |
| | PYLRYNHADAEFQERLQEDTSFGGNLGRAVFQAKKRVLEPLGLV |
| | EEGAKTAPGKKRPVEPSPQRSPDSSTGIGKKGQQPARKRLNFGQ |
| | TGDSESVPDPQPLGEPPAAPSGVGPNTMAAGGGAPMADNNEGAD |
| | GVGSSSGNWHCDSTWLGDRVITTSTRTWALPTYNNHLYKQISNG |
| | TSGGATNDNTYFGYSTPWGYFDFNRFHCHFSPRDWQRLINNNWG |
| | FRPKRLSFKLFNIQVKEVTQNEGTKTIANNLTSTIQVFTDSEYQ |
| | LPYVLGSAHQGCLPPFPADVFMIPQYGYLTLNNGSQAVGRSSFY |
| | CLEYFPSQMLRTGNNFQFTYTFEDVPFHSSYAHSQSLDRLMNPL |
| | IDQYLYYLSRTQTTGGTANTQTLGFSQGGPNTMANQAKNWLPGP |
| | CYRQQRVSTTTGQNNNSNFAWTAGTKYHLNGRNSLANPGIAMAT |
| | HKDDEERFFPSNGILIFGKQNAARDNADYSDVMLTSEEEIKTTN |
| | PVATEEYGIVADNLQQQNTAPQIGTVNSQGALPGMVWQNRDVYL |
| | QGPIWAKIPHTDGNFHPSPLMGGFGLKHPPPQILIKNTPVPADP |
| | PTTFNQSKLNSFITQYSTGQVSVEIEWELQKENSKRWNPEIQYT |
| | SNYYKSTSVDFAVNTEGVYSEPRPIGTRYLTRNL |
| AAV8 Capsid | 25 |
| Sequence (VP2) | MAPGKKRPVEPSPQRSPDSSTGIGKKGQQPARKRLNFGQTGDSE |
| | SVPDPQPLGEPPAAPSGVGPNTMAAGGGAPMADNNEGADGVGSS |
| | SGNWHCDSTWLGDRVITTSTRTWALPTYNNHLYKQISNGTSGGA |
| | TNDNTYFGYSTPWGYFDFNRFHCHFSPRDWQRLINNNWGFRPKR |
| | LSFKLFNIQVKEVTQNEGTKTIANNLTSTIQVFTDSEYQLPYVL |
| | GSAHQGCLPPFPADVFMIPQYGYLTLNNGSQAVGRSSFYCLEYF |
| | PSQMLRTGNNFQFTYTFEDVPFHSSYAHSQSLDRLMNPLIDQYL |
| | YYLSRTQTTGGTANTQTLGFSQGGPNTMANQAKNWLPGPCYRQQ |
| | RVSTTTGQNNNSNFAWTAGTKYHLNGRNSLANPGIAMATHKDDE |
| | ERFFPSNGILIFGKQNAARDNADYSDVMLTSEEEIKTTNPVATE |
| | EYGIVADNLQQQNTAPQIGTVNSQGALPGMVWQNRDVYLQGPIW |
| | AKIPHTDGNFHPSPLMGGFGLKHPPPQILIKNTPVPADPPTTFN |
| | QSKLNSFITQYSTGQVSVEIEWELQKENSKRWNPEIQYTSNYYK |
| | STSVDFAVNTEGVYSEPRPIGTRYLTRNL |
| AAV8 Capsid | 26 |
| Sequence (VP3) | MAAGGGAPMADNNEGADGVGSSSGNWHCDSTWLGDRVITTSTRT |
| | WALPTYNNHLYKQISNGTSGGATNDNTYFGYSTPWGYFDFNRFH |
| | CHFSPRDWQRLINNNWGFRPKRLSFKLFNIQVKEVTQNEGTKTI |
| | ANNLTSTIQVFTDSEYQLPYVLGSAHQGCLPPFPADVFMIPQYG |
| | YLTLNNGSQAVGRSSFYCLEYFPSQMLRTGNNFQFTYTFEDVPF |
| | HSSYAHSQSLDRLMNPLIDQYLYYLSRTQTTGGTANTQTLGFSQ |
| | GGPNTMANQAKNWLPGPCYRQQRVSTTTGQNNNSNFAWTAGTKY |
| | HLNGRNSLANPGIAMATHKDDEERFFPSNGILIFGKQNAARDNA |
| | DYSDVMLTSEEEIKTTNPVATEEYGIVADNLQQQNTAPQIGTVN |
| | SQGALPGMVWQNRDVYLQGPIWAKIPHTDGNFHPSPLMGGFGLK |
| | HPPPQILIKNTPVPADPPTTFNQSKLNSFITQYSTGQVSVEIEW |
| | ELQKENSKRWNPEIQYTSNYYKSTSVDFAVNTEGVYSEPRPIGT |
| | RYLTRNL |
| AAV9 Capsid | 27 |
| Coding Sequence | ATGGCTGCCGATGGTTATCTTCCAGATTGGCTCGAGGACAACCT |
| | TAGTGAAGGAATTCGCGAGTGGTGGGCTTTGAAACCTGGAGCCC |
| | CTCAACCCAAGGCAAATCAACAACATCAAGACAACGCTCGAGGT |
| | CTTGTGCTTCCGGGTTACAAATACCTTGGACCCGGCAACGGACT |
| | CGACAAGGGGGAGCCGGTCAACGCAGCAGACGCGGCGGCCCTCG |
| | AGCACGACAAGGCCTACGACCAGCAGCTCAAGGCCGGAGACAAC |
| | CCGTACCTCAAGTACAACCACGCCGACGCCGAGTTCCAGGAGCG |
| | GCTCAAAGAAGATACGTCTTTTGGGGGCAACCTCGGGCGAGCAG |
| | TCTTCCAGGCCAAAAAGAGGCTTCTTGAACCTCTTGGTCTGGTT |
| | GAGGAAGCGGCTAAGACGGCTCCTGGAAAGAAGAGGCCTGTAGA |
| | GCAGTCTCCTCAGGAACCGGACTCCTCCGCGGGTATTGGCAAAT |
| | CGGGTGCACAGCCCGCTAAAAAGAGACTCAATTTCGGTCAGACT |
| | GGCGACACAGAGTCAGTCCCAGACCCTCAACCAATCGGAGAACC |
| | TCCCGCAGCCCCCTCAGGTGTGGGATCTCTTACAATGGCTTCAG |
| | GTGGTGGCGCACCAGTGGCAGACAATAACGAAGGTGCCGATGGA |
| | GTGGGTAGTTCCTCGGGAAATTGGCATTGCGATTCCCAATGGCT |
| | GGGGGACAGAGTCATCACCACCAGCACCCGAACCTGGGCCCTGC |
| | CCACCTACAACAATCACCTCTACAAGCAAATCTCCAACAGCACA |
| | TCTGGAGGATCTTCAAATGACAACGCCTACTTCGGCTACAGCAC |
| | CCCCTGGGGGTATTTTGACTTCAACAGATTCCACTGCCACTTCT |
| | CACCACGTGACTGGCAGCGACTCATCAACAACAACTGGGGATTC |
| | CGGCCTAAGCGACTCAACTTCAAGCTCTTCAACATTCAGGTCAA |
| | AGAGGTTACGGACAACAATGGAGTCAAGACCATCGCCAATAACC |
| | TTACCAGCACGGTCCAGGTCTTCACGGACTCAGACTATCAGCTC |
| | CCGTACGTGCTCGGGTCGGCTCACGAGGGCTGCCTCCCGCCGTT |
| | CCCAGCGGACGTTTTCATGATTCCTCAGTACGGGTATCTGACGC |
| | TTAATGATGGAAGCCAGGCCGTGGGTCGTTCGTCCTTTTACTGC |
| | CTGGAATATTTCCCGTCGCAAATGCTAAGAACGGGTAACAACTT |
| | CCAGTTCAGCTACGAGTTTGAGAACGTACCTTTCCATAGCAGCT |
| | ACGCTCACAGCCAAAGCCTGGACCGACTAATGAATCCACTCATC |
| | GACCAATACTTGTACTATCTCTCAAAGACTATTAACGGTTCTGG |
| | ACAGAATCAACAAACGCTAAAATTCAGTGTGGCCGGACCCAGCA |
| | ACATGGCTGTCCAGGGAAGAAACTACATACCTGGACCCAGCTAC |
| | CGACAACAACGTGTCTCAACCACTGTGACTCAAAACAACAACAG |
| | CGAATTTGCTTGGCCTGGAGCTTCTTCTTGGGCTCTCAATGGAC |
| | GTAATAGCTTGATGAATCCTGGACCTGCTATGGCCAGCCACAAA |
| | GAAGGAGAGGACCGTTTCTTTCCTTTGTCTGGATCTTTAATTTT |
| | TGGCAAACAAGGAACTGGAAGAGACAACGTGGATGCGGACAAAG |
| | TCATGATAACCAACGAAGAAGAAATTAAAACTACTAACCCGGTA |
| | GCAACGGAGTCCTATGGACAAGTGGCCACAAACCACCAGAGTGC |
| | CCAAGCACAGGCGCAGACCGGCTGGGTTCAAAACCAAGGAATAC |
| | TTCCGGGTATGGTTTGGCAGGACAGAGATGTGTACCTGCAAGGA |
| | CCCATTTGGGCCAAAATTCCTCACACGGACGGCAACTTTCACCC |
| | TTCTCCGCTGATGGGAGGGTTTGGAATGAAGCACCCGCCTCCTC |
| | AGATCCTCATCAAAAACACACCTGTACCTGCGGATCCTCCAACG |
| | GCCTTCAACAAGGACAAGCTGAACTCTTTCATCACCCAGTATTC |
| | TACTGGCCAAGTCAGCGTGGAGATCGAGTGGGAGCTGCAGAAGG |
| | AAAACAGCAAGCGCTGGAACCCGGAGATCCAGTACACTTCCAAC |
| | TATTACAAGTCTAATAATGTTGAATTTGCTGTTAATACTGAAGG |
| | TGTATATAGTGAACCCCGCCCCATTGGCACCAGATACCTGACTC |
| | GTAATCTGTAA |
| AAV9 Capsid | 28 |
| Sequence (VP1) | MAADGYLPDWLEDNLSEGIREWWALKPGAPQPKANQQHQDNARG |
| | LVLPGYKYLGPGNGLDKGEPVNAADAAALEHDKAYDQQLKAGDN |
| | PYLKYNHADAEFQERLKEDTSFGGNLGRAVFQAKKRLLEPLGLV |
| | EEAAKTAPGKKRPVEQSPQEPDSSAGIGKSGAQPAKKRLNFGQT |
| | GDTESVPDPQPIGEPPAAPSGVGSLTMASGGGAPVADNNEGADG |
| | VGSSSGNWHCDSQWLGDRVITTSTRTWALPTYNNHLYKQISNST |
| | SGGSSNDNAYFGYSTPWGYFDFNRFHCHFSPRDWQRLINNNWGF |
| | RPKRLNFKLFNIQVKEVTDNNGVKTIANNLTSTVQVFTDSDYQL |
| | PYVLGSAHEGCLPPFPADVFMIPQYGYLTLNDGSQAVGRSSFYC |
| | LEYFPSQMLRTGNNFQFSYEFENVPFHSSYAHSQSLDRLMNPLI |
| | DQYLYYLSKTINGSGQNQQTLKFSVAGPSNMAVQGRNYIPGPSY |
| | RQQRVSTTVTQNNNSEFAWPGASSWALNGRNSLMNPGPAMASHK |
| | EGEDRFFPLSGSLIFGKQGTGRDNVDADKVMITNEEEIKTTNPV |
| | ATESYGQVATNHQSAQAQAQTGWVQNQGILPGMVWQDRDVYLQG |
| | PIWAKIPHTDGNFHPSPLMGGFGMKHPPPQILIKNTPVPADPPT |
| | AFNKDKLNSFITQYSTGQVSVEIEWELQKENSKRWNPEIQYTSN |
| | YYKSNNVEFAVNTEGVYSEPRPIGTRYLTRNL |
| AAV9 Capsid | 29 |
| Sequence (VP2) | TAPGKKRPVEQSPQEPDSSAGIGKSGAQPAKKRLNFGQTGDTES |
| | VPDPQPIGEPPAAPSGVGSLTMASGGGAPVADNNEGADGVGSSS |
| | GNWHCDSQWLGDRVITTSTRTWALPTYNNHLYKQISNSTSGGSS |
| | NDNAYFGYSTPWGYFDFNRFHCHFSPRDWQRLINNNWGFRPKRL |
| | NFKLFNIQVKEVTDNNGVKTIANNLTSTVQVFTDSDYQLPYVLG |
| | SAHEGCLPPFPADVFMIPQYGYLTLNDGSQAVGRSSFYCLEYFP |
| | SQMLRTGNNFQFSYEFENVPFHSSYAHSQSLDRLMNPLIDQYLY |
| | YLSKTINGSGQNQQTLKFSVAGPSNMAVQGRNYIPGPSYRQQRV |
| | STTVTQNNNSEFAWPGASSWALNGRNSLMNPGPAMASHKEGEDR |
| | FFPLSGSLIFGKQGTGRDNVDADKVMITNEEEIKTTNPVATESY |
| | GQVATNHQSAQAQAQTGWVQNQGILPGMVWQDRDVYLQGPIWAK |
| | IPHTDGNFHPSPLMGGFGMKHPPPQILIKNTPVPADPPTAFNKD |
| | KLNSFITQYSTGQVSVEIEWELQKENSKRWNPEIQYTSNYYKSN |
| | NVEFAVNTEGVYSEPRPIGTRYLTRNL |
| AAV9 Capsid | 30 |
| Sequence (VP3) | MASGGGAPVADNNEGADGVGSSSGNWHCDSQWLGDRVITTSTRT |
| | WALPTYNNHLYKQISNSTSGGSSNDNAYFGYSTPWGYFDFNRFH |
| | CHFSPRDWQRLINNNWGFRPKRLNFKLFNIQVKEVTDNNGVKTI |
| | ANNLTSTVQVFTDSDYQLPYVLGSAHEGCLPPFPADVFMIPQYG |
| | YLTLNDGSQAVGRSSFYCLEYFPSQMLRTGNNFQFSYEFENVPF |
| | HSSYAHSQSLDRLMNPLIDQYLYYLSKTINGSGQNQQTLKFSVA |
| | GPSNMAVQGRNYIPGPSYRQQRVSTTVTQNNNSEFAWPGASSWA |
| | LNGRNSLMNPGPAMASHKEGEDRFFPLSGSLIFGKQGTGRDNVD |
| | ADKVMITNEEEIKTTNPVATESYGQVATNHQSAQAQAQTGWVQN |
| | QGILPGMVWQDRDVYLQGPIWAKIPHTDGNFHPSPLMGGFGMKH |
| | PPPQILIKNTPVPADPPTAFNKDKLNSFITQYSTGQVSVEIEWE |
| | LQKENSKRWNPEIQYTSNYYKSNNVEFAVNTEGVYSEPRPIGTR |
| | YLTRNL |
| AAV2 Capsid | 31 |
| Coding Sequence | ATGGCTGCCGATGGTTATCTTCCAGATTGGCTCGAGGACACTCT |
| | CTCTGAAGGAATAAGACAGTGGTGGAAGCTCAAACCTGGCCCAC |
| | CACCACCAAAGCCCGCAGAGCGGCATAAGGACGACAGCAGGGGT |
| | CTTGTGCTTCCTGGGTACAAGTACCTCGGACCCTTCAACGGACT |
| | CGACAAGGGAGAGCCGGTCAACGAGGCAGACGCCGCGGCCCTCG |
| | AGCACGACAAAGCCTACGACCGGCAGCTCGACAGCGGAGACAAC |
| | CCGTACCTCAAGTACAACCACGCCGACGCGGAGTTTCAGGAGCG |
| | CCTTAAAGAAGATACGTCTTTTGGGGGCAACCTCGGACGAGCAG |
| | TCTTCCAGGCGAAAAAGAGGGTTCTTGAACCTCTGGGCCTGGTT |
| | GAGGAACCTGTTAAGACGGCTCCGGGAAAAAAGAGGCCGGTAGA |
| | GCACTCTCCTGTGGAGCCAGACTCCTCCTCGGGAACCGGAAAGG |
| | CGGGCCAGCAGCCTGCAAGAAAAAGATTGAATTTTGGTCAGACT |
| | GGAGACGCAGACTCAGTACCTGACCCCCAGCCTCTCGGACAGCC |
| | ACCAGCAGCCCCCTCTGGTCTGGGAACTAATACGATGGCTACAG |
| | GCAGTGGCGCACCAATGGCAGACAATAACGAGGGCGCCGACGGA |
| | GTGGGTAATTCCTCGGGAAATTGGCATTGCGATTCCACATGGAT |
| | GGGCGACAGAGTCATCACCACCAGCACCCGAACCTGGGCCCTGC |
| | CCACCTACAACAACCACCTCTACAAACAAATTTCCAGCCAATCA |
| | GGAGCCTCGAACGACAATCACTACTTTGGCTACAGCACCCCTTG |
| | GGGGTATTTTGACTTCAACAGATTCCACTGCCACTTTTCACCAC |
| | GTGACTGGCAAAGACTCATCAACAACAACTGGGGATTCCGACCC |
| | AAGAGACTCAACTTCAAGCTCTTTAACATTCAAGTCAAAGAGGT |
| | CACGCAGAATGACGGTACGACGACGATTGCCAATAACCTTACCA |
| | GCACGGTTCAGGTGTTTACTGACTCGGAGTACCAGCTCCCGTAC |
| | GTCCTCGGCTCGGCGCATCAAGGATGCCTCCCGCCGTTCCCAGC |
| | AGACGTCTTCATGGTGCCACAGTATGGATACCTCACCCTGAACA |
| | ACGGGAGTCAGGCAGTAGGACGCTCTTCATTTTACTGCCTGGAG |
| | TACTTTCCTTCTCAGATGCTGCGTACCGGAAACAACTTTACCTT |
| | CAGCTACACTTTTGAGGACGTTCCTTTCCACAGCAGCTACGCTC |
| | ACAGCCAGAGTCTGGACCGTCTCATGAATCCTCTCATCGACCAG |
| | TACCTGTATTACTTGAGCAGAACAAACACTCCAAGTGGAACCAC |
| | CACGCAGTCAAGGCTTCAGTTTTCTCAGGCCGGAGCGAGTGACA |
| | TTCGGGACCAGTCTAGGAACTGGCTTCCTGGACCCTGTTACCGC |
| | CAGCAGCGAGTATCAAAGACATCTGCGGATAACAACAACAGTGA |
| | ATACTCGTGGACTGGAGCTACCAAGTACCACCTCAATGGCAGAG |
| | ACTCTCTGGTGAATCCGGGCCCGGCCATGGCAAGCCACAAGGAC |
| | GATGAAGAAAAGTTTTTTCCTCAGAGCGGGGTTCTCATCTTTGG |
| | GAAGCAAGGCTCAGAGAAAACAAATGTGGACATTGAAAAGGTCA |
| | TGATTACAGACGAAGAGGAAATCAGGACAACCAATCCCGTGGCT |
| | ACGGAGCAGTATGGTTCTGTATCTACCAACCTCCAGAGAGGCAA |
| | CAGACAAGCAGCTACCGCAGATGTCAACACACAAGGCGTTCTTC |
| | CAGGCATGGTCTGGCAGGACAGAGATGTGTACCTTCAGGGGCCC |
| | ATCTGGGCAAAGATTCCACACACGGACGGACATTTTCACCCCTC |
| | TCCCCTCATGGGTGGATTCGGACTTAAACACCCTCCTCCACAGA |
| | TTCTCATCAAGAACACCCCGGTACCTGCGAATCCTTCGACCACC |
| | TTCAGTGCGGCAAAGTTTGCTTCCTTCATCACACAGTACTCCAC |
| | GGGACAGGTCAGCGTGGAGATCGAGTGGGAGCTGCAGAAGGAAA |
| | ACAGCAAACGCTGGAATCCCGAAATTCAGTACACTTCCAACTAC |
| | AACAAGTCTGTTAATGTGGACTTTACTGTGGACACTAATGGCGT |
| | GTATTCAGAGCCTCGCCCCATTGGCACCAGATACCTGACTCGTA |
| | ATCTGTAA |
| AAV2 Capsid | 32 |
| Sequence (VP1) | MAADGYLPDWLEDTLSEGIRQWWKLKPGPPPPKPAERHKDDSRG |
| | LVLPGYKYLGPFNGLDKGEPVNEADAAALEHDKAYDRQLDSGDN |
| | PYLKYNHADAEFQERLKEDTSFGGNLGRAVFQAKKRVLEPLGLV |
| | EEPVKTAPGKKRPVEHSPVEPDSSSGTGKAGQQPARKRLNFGQT |
| | GDADSVPDPQPLGQPPAAPSGLGTNTMATGSGAPMADNNEGADG |
| | VGNSSGNWHCDSTWMGDRVITTSTRTWALPTYNNHLYKQISSQS |
| | GASNDNHYFGYSTPWGYFDFNRFHCHFSPRDWQRLINNNWGFRP |
| | KRLNFKLFNIQVKEVTQNDGTTTIANNLTSTVQVFTDSEYQLPY |
| | VLGSAHQGCLPPFPADVFMVPQYGYLTLNNGSQAVGRSSFYCLE |
| | YFPSQMLRTGNNFTFSYTFEDVPFHSSYAHSQSLDRLMNPLIDQ |
| | YLYYLSRTNTPSGTTTQSRLQFSQAGASDIRDQSRNWLPGPCYR |
| | QQRVSKTSADNNNSEYSWTGATKYHLNGRDSLVNPGPAMASHKD |
| | DEEKFFPQSGVLIFGKQGSEKTNVDIEKVMITDEEEIRTTNPVA |
| | TEQYGSVSTNLQRGNRQAATADVNTQGVLPGMVWQDRDVYLQGP |
| | IWAKIPHTDGHFHPSPLMGGFGLKHPPPQILIKNTPVPANPSTT |
| | FSAAKFASFITQYSTGQVSVEIEWELQKENSKRWNPEIQYTSNY |
| | NKSVNVDFTVDTNGVYSEPRPIGTRYLTRNL |
| AAV2 Capsid | 33 |
| Sequence (VP2) | MAPGKKRPVEHSPVEPDSSSGTGKAGQQPARKRLNFGQTGDADS |
| | VPDPQPLGQPPAAPSGLGTNTMATGSGAPMADNNEGADGVGNSS |
| | GNWHCDSTWMGDRVITTSTRTWALPTYNNHLYKQISSQSGASND |
| | NHYFGYSTPWGYFDFNRFHCHFSPRDWQRLINNNWGFRPKRLNF |
| | KLFNIQVKEVTQNDGTTTIANNLTSTVQVFTDSEYQLPYVLGSA |
| | HQGCLPPFPADVFMVPQYGYLTLNNGSQAVGRSSFYCLEYFPSQ |
| | MLRTGNNFTFSYTFEDVPFHSSYAHSQSLDRLMNPLIDQYLYYL |
| | SRTNTPSGTTTQSRLQFSQAGASDIRDQSRNWLPGPCYRQQRVS |
| | KTSADNNNSEYSWTGATKYHLNGRDSLVNPGPAMASHKDDEEKF |
| | FPQSGVLIFGKQGSEKTNVDIEKVMITDEEEIRTTNPVATEQYG |
| | SVSTNLQRGNRQAATADVNTQGVLPGMVWQDRDVYLQGPIWAKI |
| | PHTDGHFHPSPLMGGFGLKHPPPQILIKNTPVPANPSTTFSAAK |
| | FASFITQYSTGQVSVEIEWELQKENSKRWNPEIQYTSNYNKSVN |
| | VDFTVDTNGVYSEPRPIGTRYLTRNL |
| AAV2 Capsid | 34 |
| Sequence (VP3) | MATGSGAPMADNNEGADGVGNSSGNWHCDSTWMGDRVITTSTRT |
| | WALPTYNNHLYKQISSQSGASNDNHYFGYSTPWGYFDFNRFHCH |
| | FSPRDWQRLINNNWGFRPKRLNFKLFNIQVKEVTQNDGTTTIAN |
| | NLTSTVQVFTDSEYQLPYVLGSAHQGCLPPFPADVFMVPQYGYL |
| | TLNNGSQAVGRSSFYCLEYFPSQMLRTGNNFTFSYTFEDVPFHS |
| | SYAHSQSLDRLMNPLIDQYLYYLSRTNTPSGTTTQSRLQFSQAG |
| | ASDIRDQSRNWLPGPCYRQQRVSKTSADNNNSEYSWTGATKYHL |
| | NGRDSLVNPGPAMASHKDDEEKFFPQSGVLIFGKQGSEKTNVDI |
| | EKVMITDEEEIRTTNPVATEQYGSVSTNLQRGNRQAATADVNTQ |
| | GVLPGMVWQDRDVYLQGPIWAKIPHTDGHFHPSPLMGGFGLKHP |
| | PPQILIKNTPVPANPSTTFSAAKFASFITQYSTGQVSVEIEWEL |
| | QKENSKRWNPEIQYTSNYNKSVNVDFTVDTNGVYSEPRPIGTRY |
| | LTRNL |
| AAV5 Capsid | 35 |
| Coding Sequence | ATGTCTTTTGTTGATCACCCTCCAGATTGGTTGGAAGAAGTTGG |
| | TGAAGGTCTTCGCGAGTTTTTGGGCCTTGAAGCGGGCCCACCGA |
| | AACCAAAACCCAATCAGCAGCATCAAGATCAAGCCCGTGGTCTT |
| | GTGCTGCCTGGTTATAACTATCTCGGACCCGGAAACGGTCTCGA |
| | TCGAGGAGAGCCTGTCAACAGGGCAGACGAGGTCGCGCGAGAGC |
| | ACGACATCTCGTACAACGAGCAGCTTGAGGCGGGAGACAACCCC |
| | TACCTCAAGTACAACCACGCGGACGCCGAGTTTCAGGAGAAGCT |
| | CGCCGACGACACATCCTTCGGGGGAAACCTCGGAAAGGCAGTCT |
| | TTCAGGCCAAGAAAAGGGTTCTCGAACCTTTTGGCCTGGTTGAA |
| | GAGGGTGCTAAGACGGCCCCTACCGGAAAGCGGATAGACGACCA |
| | CTTTCCAAAAAGAAAGAAGGCTCGGACCGAAGAGGACTCCAAGC |
| | CTTCCACCTCGTCAGACGCCGAAGCTGGACCCAGCGGATCCCAG |
| | CAGCTGCAAATCCCAGCCCAACCAGCCTCAAGTTTGGGAGCTGA |
| | TACAATGTCTGCGGGAGGTGGCGGCCCATTGGGCGACAATAACC |
| | AAGGTGCCGATGGAGTGGGCAATGCCTCGGGAGATTGGCATTGC |
| | GATTCCACGTGGATGGGGGACAGAGTCGTCACCAAGTCCACCCG |
| | AACCTGGGTGCTGCCCAGCTACAACAACCACCAGTACCGAGAGA |
| | TCAAAAGCGGCTCCGTCGACGGAAGCAACGCCAACGCCTACTTT |
| | GGATACAGCACCCCCTGGGGGTACTTTGACTTTAACCGCTTCCA |
| | CAGCCACTGGAGCCCCCGAGACTGGCAAAGACTCATCAACAACT |
| | ACTGGGGCTTCAGACCCCGGTCCCTCAGAGTCAAAATCTTCAAC |
| | ATTCAAGTCAAAGAGGTCACGGTGCAGGACTCCACCACCACCAT |
| | CGCCAACAACCTCACCTCCACCGTCCAAGTGTTTACGGACGACG |
| | ACTACCAGCTGCCCTACGTCGTCGGCAACGGGACCGAGGGATGC |
| | CTGCCGGCCTTCCCTCCGCAGGTCTTTACGCTGCCGCAGTACGG |
| | TTACGCGACGCTGAACCGCGACAACACAGAAAATCCCACCGAGA |
| | GGAGCAGCTTCTTCTGCCTAGAGTACTTTCCCAGCAAGATGCTG |
| | AGAACGGGCAACAACTTTGAGTTTACCTACAACTTTGAGGAGGT |
| | GCCCTTCCACTCCAGCTTCGCTCCCAGTCAGAACCTCTTCAAGC |
| | TGGCCAACCCGCTGGTGGACCAGTACTTGTACCGCTTCGTGAGC |
| | ACAAATAACACTGGCGGAGTCCAGTTCAACAAGAACCTGGCCGG |
| | GAGATACGCCAACACCTACAAAAACTGGTTCCCGGGGCCCATGG |
| | GCCGAACCCAGGGCTGGAACCTGGGCTCCGGGGTCAACCGCGCC |
| | AGTGTCAGCGCCTTCGCCACGACCAATAGGATGGAGCTCGAGGG |
| | CGCGAGTTACCAGGTGCCCCCGCAGCCGAACGGCATGACCAACA |
| | ACCTCCAGGGCAGCAACACCTATGCCCTGGAGAACACTATGATC |
| | TTCAACAGCCAGCCGGCGAACCCGGGCACCACCGCCACGTACCT |
| | CGAGGGCAACATGCTCATCACCAGCGAGAGCGAGACGCAGCCGG |
| | TGAACCGCGTGGCGTACAACGTCGGCGGGCAGATGGCCACCAAC |
| | AACCAGAGCTCCACCACTGCCCCCGCGACCGGCACGTACAACCT |
| | CCAGGAAATCGTGCCCGGCAGCGTGTGGATGGAGAGGGACGTGT |
| | ACCTCCAAGGACCCATCTGGGCCAAGATCCCAGAGACGGGGGCG |
| | CACTTTCACCCCTCTCCGGCCATGGGCGGATTCGGACTCAAACA |
| | CCCACCGCCCATGATGCTCATCAAGAACACGCCTGTGCCCGGAA |
| | ATATCACCAGCTTCTCGGACGTGCCCGTCAGCAGCTTCATCACC |
| | CAGTACAGCACCGGGCAGGTCACCGTGGAGATGGAGTGGGAGCT |
| | CAAGAAGGAAAACTCCAAGAGGTGGAACCCAGAGATCCAGTACA |
| | CAAACAACTACAACGACCCCCAGTTTGTGGACTTTGCCCCGGAC |
| | AGCACCGGGGAATACAGAACCACCAGACCTATCGGAACCCGATA |
| | CCTTACCCGACCCCTTTAA |
| AAV5 Capsid | 36 |
| Sequence (VP1) | MSFVDHPPDWLEEVGEGLREFLGLEAGPPKPKPNQQHQDQARGL |
| | VLPGYNYLGPGNGLDRGEPVNRADEVAREHDISYNEQLEAGDNP |
| | YLKYNHADAEFQEKLADDTSFGGNLGKAVFQAKKRVLEPFGLVE |
| | EGAKTAPTGKRIDDHFPKRKKARTEEDSKPSTSSDAEAGPSGSQ |
| | QLQIPAQPASSLGADTMSAGGGGPLGDNNQGADGVGNASGDWHC |
| | DSTWMGDRVVTKSTRTWVLPSYNNHQYREIKSGSVDGSNANAYF |
| | GYSTPWGYFDFNRFHSHWSPRDWQRLINNYWGFRPRSLRVKIFN |
| | IQVKEVTVQDSTTTIANNLTSTVQVFTDDDYQLPYVVGNGTEGC |
| | LPAFPPQVFTLPQYGYATLNRDNTENPTERSSFFCLEYFPSKML |
| | RTGNNFEFTYNFEEVPFHSSFAPSQNLFKLANPLVDQYLYRFVS |
| | TNNTGGVQFNKNLAGRYANTYKNWFPGPMGRTQGWNLGSGVNRA |
| | SVSAFATTNRMELEGASYQVPPQPNGMTNNLQGSNTYALENTMI |
| | FNSQPANPGTTATYLEGNMLITSESETQPVNRVAYNVGGQMATN |
| | NQSSTTAPATGTYNLQEIVPGSVWMERDVYLQGPIWAKIPETGA |
| | HFHPSPAMGGFGLKHPPPMMLIKNTPVPGNITSFSDVPVSSFIT |
| | QYSTGQVTVEMEWELKKENSKRWNPEIQYTNNYNDPQFVDFAPD |
| | STGEYRTTRPIGTRYLTRPL |
| AAV5 Capsid | 37 |
| Sequence (VP2) | TAPTGKRIDDHFPKRKKARTEEDSKPSTSSDAEAGPSGSQQLQI |
| | PAQPASSLGADTMSAGGGGPLGDNNQGADGVGNASGDWHCDSTW |
| | MGDRVVTKSTRTWVLPSYNNHQYREIKSGSVDGSNANAYFGYST |
| | PWGYFDFNRFHSHWSPRDWQRLINNYWGFRPRSLRVKIFNIQVK |
| | EVTVQDSTTTIANNLTSTVQVFTDDDYQLPYVVGNGTEGCLPAF |
| | PPQVFTLPQYGYATLNRDNTENPTERSSFFCLEYFPSKMLRTGN |
| | NFEFTYNFEEVPFHSSFAPSQNLFKLANPLVDQYLYRFVSTNNT |
| | GGVQFNKNLAGRYANTYKNWFPGPMGRTQGWNLGSGVNRASVSA |
| | FATTNRMELEGASYQVPPQPNGMTNNLQGSNTYALENTMIFNSQ |
| | PANPGTTATYLEGNMLITSESETQPVNRVAYNVGGQMATNNQSS |
| | TTAPATGTYNLQEIVPGSVWMERDVYLQGPIWAKIPETGAHFHP |
| | SPAMGGFGLKHPPPMMLIKNTPVPGNITSFSDVPVSSFITQYST |
| | GQVTVEMEWELKKENSKRWNPEIQYTNNYNDPQFVDFAPDSTGE |
| | YRTTRPIGTRYLTRPL |
| AAV5 Capsid | 38 |
| Sequence (VP3) | MSAGGGGPLGDNNQGADGVGNASGDWHCDSTWMGDRVVTKSTRT |
| | WVLPSYNNHQYREIKSGSVDGSNANAYFGYSTPWGYFDFNRFHS |
| | HWSPRDWQRLINNYWGFRPRSLRVKIFNIQVKEVTVQDSTTTIA |
| | NNLTSTVQVFTDDDYQLPYVVGNGTEGCLPAFPPQVFTLPQYGY |
| | ATLNRDNTENPTERSSFFCLEYFPSKMLRTGNNFEFTYNFEEVP |
| | FHSSFAPSQNLFKLANPLVDQYLYRFVSTNNTGGVQFNKNLAGR |
| | YANTYKNWFPGPMGRTQGWNLGSGVNRASVSAFATTNRMELEGA |
| | SYQVPPQPNGMTNNLQGSNTYALENTMIFNSQPANPGTTATYLE |
| | GNMLITSESETQPVNRVAYNVGGQMATNNQSSTTAPATGTYNLQ |
| | EIVPGSVWMERDVYLQGPIWAKIPETGAHFHPSPAMGGFGLKHP |
| | PPMMLIKNTPVPGNITSFSDVPVSSFITQYSTGQVTVEMEWELK |
| | KENSKRWNPEIQYTNNYNDPQFVDFAPDSTGEYRTTRPIGTRYL |
| | TRPL |
| Macaca mulatta | 39 |
| (RHESUS MONKEY) | ATGGCGGGCATCTGGCTGGGGCTCGTGTGGCAGAAGCTGCTGCT |
| CYP4V2 CDS | GTGGGGCGCGGCGAGTGCCGTGTCCCTGGCCGGCGCCAGTCTGG |
| NM_001193838.1 | TCCTGAGCCTGCTGCAGAGGGTGGCGAGCTACGTGAGGAAATGG |
| | CAGCAGATGCGGCCCATCCCCACGGTGGCCCGCGCCTACCCACT |
| | GGTGGGCCACGCGCTGCTGATGAAGCGGGACGGGCGAGAATTTT |
| | TTCAGCAGATCATTGAGTACACAGAGGAATACCGCCACATGCCA |
| | CTCCTGAAGCTCTGGGTCGGGCCGGTGCCCATGGTGGCCCTTTA |
| | TAATGCAGAAAATGTGGAGGTAATTTTAACTAGTTCAAAGCAAA |
| | TTGACAAATCCTCTATGTACAAGTTTTTAGAACCATGGCTTGGC |
| | CTAGGACTTCTTACAAGTACTGGAAACAAATGGCGCTCCAGGAG |
| | AAAGATGTTAACACCCACTTTCCATTTTACCATTCTGGAAGATT |
| | TCTTAGATATCATGAATGAACAAGCAAATATATTGGTTAAGAAA |
| | CTTGAAAAACATGTTAACCAAGAAGCATTTAACTGCTTTGTTTA |
| | CATCACTCTTTGTGCCTTAGATATCATCTGTGAAACAGCTATGG |
| | GGAAGAATATTGGTGCTCAAAGTAACGATGATTCCGAGTATGTC |
| | CGTGCAGTTTATAGAATGAGTGAGATGATATTTCGAAGAATAAA |
| | GATGCCGTGGCTTTGGCTTGACCTCTGGTACCTTATGTTTAAAG |
| | AGGGATGGGAACACAAAAAGAGCCTTAAGATCCTACATGCTTTT |
| | ACCAACAATGTTATCGCTGAACGGGCCAATGAAATGAACGTGGA |
| | TGAAGACTGTAGAGGTGATGGCAGGGACTCCGCCCCCTCCAAAA |
| | ATAAACGCAGGGCCTTTCTTGACTTGCTTTTAAGTGTGACTGAC |
| | GACGAAGGGAACAGGCTAAGTCATGAAGATATTCGAGAAGAAGT |
| | TGACACCTTCATGTTTGAGGGCCACGACACAACTGCAGCTGCAA |
| | TGAACTGGTCCTTATACCTGTTGGGGTCTAACCCAGAAGTCCAG |
| | AAAAAAGTGGACCATGAACTGGATGACGTGTTTGGGAGGTCTGA |
| | CCGTCCCGCTACTGTAGAAGACCTGAAGAAACTTCGGTATCTGG |
| | AATGTGTTATTAAGGAGACCCTTCGCCTTTTTCCTTCTGTTCCT |
| | TTATTTGCCCGCAGTGTTAGTGAAGATTGTGAAGTGGCAGGTTA |
| | CAGAGTTCTGAAAGGCACTGAAGCCGTCATCATTCCCTATGCAT |
| | TGCATAGAGATCCAAGATACTTCCCCAACCCTGAGGAGTTCCGG |
| | CCTGAGCGGTTCTTCCCCGAGAATGCACAAGGGCGCCATCCATA |
| | TGCCTACGTGCCCTTCTCTGCTGGCCCCAGGAACTGTATAGGTC |
| | AAAAGTTTGCTGTGATGGAAGAAAAGACCATTCTTTCGTGCATC |
| | CTAAGGCACTTTTGGATAGAATCCAACCAGAAAAGAGAAGAACT |
| | TGGTCTAGAAGGACAGTTGATTCTTCGTCCAACTAATGGCATCT |
| | GGATCAAGTTGAAGAGGAGAAATGCAGATGAACCCTAA |
| Macaca mulatta | 40 |
| (RHESUS MONKEY) | MAGIWLGLVWQKLLLWGAASAVSLAGASLVLSLLQRVASYVRKW |
| CYP4V2 GENE | QQMRPIPTVARAYPLVGHALLMKRDGREFFQQIIEYTEEYRHMP |
| PRODUCT | LLKLWVGPVPMVALYNAENVEVILTSSKQIDKSSMYKFLEPWLG |
| NP_001180767.1 | LGLLTSTGNKWRSRRKMLTPTFHFTILEDFLDIMNEQANILVKK |
| | LEKHVNQEAFNCFVYITLCALDIICETAMGKNIGAQSNDDSEYV |
| | RAVYRMSEMIFRRIKMPWLWLDLWYLMFKEGWEHKKSLKILHAF |
| | TNNVIAERANEMNVDEDCRGDGRDSAPSKNKRRAFLDLLLSVTD |
| | DEGNRLSHEDIREEVDTFMFEGHDTTAAAMNWSLYLLGSNPEVQ |
| | KKVDHELDDVFGRSDRPATVEDLKKLRYLECVIKETLRLFPSVP |
| | LFARSVSEDCEVAGYRVLKGTEAVIIPYALHRDPRYFPNPEEFR |
| | PERFFPENAQGRHPYAYVPFSAGPRNCIGQKFAVMEEKTILSCI |
| | LRHFWIESNQKREELGLEGQLILRPTNGIWIKLKRRNADEP |
| Bos taurus | 41 |
| CYP4V2 CDS | ATGCTGGCGCCGTGGTTGCTGAGCGTCGGGCCGAAGCTGCTGCT |
| NM_001034373.2 | CTGGAGCGGGCTGTGCGCCGTCTCCCTGGCAGGCGCCACCCTCA |
| | CCCTGAACCTCCTGAAGATGGTGGCGAGCTATGCGCGGAAATGG |
| | CGTCAGATGCGTCCCGTCCCGACCATTGGGGACCCCTACCCCTT |
| | GGTGGGACACGCGCTGATGATGAAGCCCGATGCAAGAGATTTTT |
| | TTCAGCAGATAATTGATTTCACTGAAGAATGCCGACACCTGCCA |
| | CTGCTGAAACTCTGGCTCGGGCCTGTGCCTCTCGTGGCCCTTTA |
| | TAACGCAGAAACTGTGGAAGTAATTTTAAGCAGTTCAAAGCACA |
| | TTGAAAAATCCTATATGTACAAGTTCTTAGAACCGTGGCTTGGA |
| | CTAGGACTTCTTACAAGTACTGGAAACAAATGGCGATCTAGGAG |
| | AAAAATGTTAACACCCACTTTCCATTTTACAATTCTGGAGGATT |
| | TCTTAGATGTCATGAATGAACAAGCAAATATATTGGTTACTAAG |
| | CTTGAAAAGCATGTTAACCAAGAAGCGTTTAACTGCTTTTTTTA |
| | CGTCACTCTTTGTACCTTAGATATAATCTGTGAAACAGCTATGG |
| | GAAAGAACATTGGTGCTCAAAGAAATGATGATTCCGAGTATGTT |
| | CGAGCCGTTTATAGGATGAGTGATTCGATACATCAAAGAATGAA |
| | GATGCCCTGGCTCTGGCTTGACCTTATATTCTATATGTTTAAAA |
| | ATGGACGAGAACACAGAAGGAGCCTAAAGATTGTACATGATTTT |
| | ACCAACAATGTCATCACTGAACGGGCCAATGAAATGAAGAGACA |
| | TGAAGAAGGTACGAGTAACGACAAGGAGAAGGACTTTCCTCCAC |
| | GCAAAACTAAATGCAGGGCTTTTCTTGACTTGCTTTTAAATGTG |
| | ACTGATGACCAAGGGAACAAGCTGAGTCATGAAGATATAAGAGA |
| | AGAAGTCGACACCTTTATGTTTGAGGGCCATGATACAACTGCAG |
| | CTGCAATAAACTGGTCCTTGTATCTGTTGGGTTGGTATCCAGAA |
| | GTCCAGCAGAGAGTGGACACTGAGCTGGAAGAAGTGTTTGGGAA |
| | GTCTGACCGTCCTGTTACCCTAGAAGACCTGAAGAAACTTAAAT |
| | ATCTGGACTGTGTTATTAAGGAGAGCCTTCGCCTTTTCCCGTCT |
| | GTTCCTTTCTTTGCCCGTAATCTTACCGAAGACTGTGAAGTTGC |
| | GGGTCACAAAATCGTGCAAGGCTGTCAAGTAATCATTGTGCCCT |
| | ACGCACTGCATAGAGATCCAAAGTACTTCCCGGATCCTGAGGAA |
| | TTCAAGCCAGAACGGTTCTTTCCCGAGAATTTGAAAGGACGTCA |
| | TACATACGCATATGTGCCCTTCTCTGCAGGCCCCCGAAACTGTA |
| | TAGGTCAAAAGTTTGCCATAATGGAAGAAAAGACCATTCTTTCC |
| | TGCATCCTTAGGCACTTTTGGGTAGAATCCAACCAAAAAAGAGA |
| | AGAACTTGGTCTAGCAGGAGAGCTCATTCTTCGTCCAAGTAACG |
| | GCATCTGGATCAAGTTGAAGAGGAGAAACACAGATGAATCCTAA |
| Bos taurus | 42 |
| CYP4V2 GENE | MLAPWLLSVGPKLLLWSGLCAVSLAGATLTLNLLKMVASYARKW |
| PRODUCT | RQMRPVPTIGDPYPLVGHALMMKPDARDFFQQIIDFTEECRHLP |
| NP_001029545.1 | LLKLWLGPVPLVALYNAETVEVILSSSKHIEKSYMYKFLEPWLG |
| | LGLLTSTGNKWRSRRKMLTPTFHFTILEDFLDVMNEQANILVTK |
| | LEKHVNQEAFNCFFYVTLCTLDIICETAMGKNIGAQRNDDSEYV |
| | RAVYRMSDSIHQRMKMPWLWLDLIFYMFKNGREHRRSLKIVHDF |
| | TNNVITERANEMKRHEEGTSNDKEKDFPPRKTKCRAFLDLLLNV |
| | TDDQGNKLSHEDIREEVDTFMFEGHDTTAAAINWSLYLLGWYPE |
| | VQQRVDTELEEVFGKSDRPVTLEDLKKLKYLDCVIKESLRLFPS |
| | VPFFARNLTEDCEVAGHKIVQGCQVIIVPYALHRDPKYFPDPEE |
| | FKPERFFPENLKGRHTYAYVPFSAGPRNCIGQKFAIMEEKTILS |
| | CILRHFWVESNQKREELGLAGELILRPSNGIWIKLKRRNTDES |
| Rattus | 43 |
| norvegicus | ATGTTGTGGCTGTGGTTAGGGCTCAGCGGGCAGAAGCTATTGCT |
| CYP4V3 CDS | TTGGGGCGCAGCGAGCGCGGTCTCCGTGGCCGGCGCCACTGTCT |
| NM_001135600.1 | TGCTCAACATCCTGCAGATGTTGGTAAGCTATGCACGAAAGTGG |
| | CAGCAGATGCGGCCAATCCCGTCGGTGGCTCGCGCTTACCCCTT |
| | GGTGGGACATGCGCTGTTTATGAAGCCCAACAACACAGAATTTT |
| | TTCAGCAGATAATTCAGTACACAGAAGAATTCCGACACCTGCCC |
| | ATCATTAAACTCTGGATTGGACCAGTGCCCCTGGTGGCACTTTA |
| | TAAGGCAGAGAATGTGGAGGTGATTTTGACCAGTTCGAAGCAAA |
| | TTGATAAATCCTTTATGTACAAGTTCCTACAGCCATGGCTGGGA |
| | CTAGGACTTCTTACAAGTACTGGGAGCAAATGGCGTGCCAGGAG |
| | GAAGATGTTAACACCCAGTTTCCATTTTACAATTCTGGAGGATT |
| | TCTTAGATGTCATGAATGAGCAAGCAAATATATTGGTTAACAAG |
| | CTTGAAAAACATGTCAATCAAGAGGCCTTTAACTGCTTTTTCCC |
| | CATCACTCTTTGTGCTCTGGATATAATCTGTGAAACGGCTATGG |
| | GGAAGAACATTGGAGCTCAAAGTAATGGTGATTCTGAGTATGTC |
| | CGTACAGTGTATAGGATGAGCGATATGATATACAGAAGAATGAA |
| | GATGCCCTGGTTTTGGTTTGACCTTTGGTACCTTATGTTTAAAG |
| | AAGGAAGGGACCACAAAAAGGGACTAAAGAGTCTACATACTTTT |
| | ACCAACAATGTCATTGCTGAACGGGTTAATGCAAGGAAGGCAGA |
| | GCAAGACTGCATAGGTGCTGGGAGGGGTCCTCTCCCCTCGAAAA |
| | CTAAGCGCAAGGCCTTTCTTGACTTGCTTTTGAGTGTGACTGAT |
| | GAGGAAGGAAACAAATTAAGCCATGAAGACATCCGAGAGGAAGT |
| | TGACACCTTCATGTTTGAGGGTCACGATACAACTGCTGCTGCCA |
| | TAAACTGGTCCTTATACCTCCTGGGCTCTAATCCAGAAGTCCAG |
| | AGGAAAGTGGACAAGGAGCTGGATGATGTGTTTGGAAGATCCCA |
| | TCGCCCTGTCACCTTGGAAGACCTGAAGAAACTTAAATATCTGG |
| | ATTGTGTCATTAAGGAGACCCTCCGTGTTTTCCCATCTGTCCCT |
| | TTATTTGCCCGGAGTCTTAGCGAAGACTGTGAAGTGGCGGGTTA |
| | CAAAATCTCAAAAGGAACGGAAGCAGTCATCATTCCCTATGCAC |
| | TACATCGAGACCCTAGATACTTCCCAGACCCTGAGGAATTCCAG |
| | CCAGAGCGGTTCTTTCCTGAAAACTCCCAAGGACGCCACCCCTA |
| | TGCCTATGTGCCATTCTCTGCTGGACCTAGAAACTGCATTGGTC |
| | AAAAGTTTGCGGTCATGGAGGAAAAGACCATTCTTGCCTGTATC |
| | CTGAGGGAGTTTTGGATAGAATCCAACCAGAAGAGAGAAGAACT |
| | CGGCCTGGCTGGAGATTTGATTCTTAGGCCAAATAATGGCATCT |
| | GGATCAAGCTGAAGAGGAGGCATGAAGATGACCCCTAA |
| Rattus | 44 |
| norvegicus | MLWLWLGLSGQKLLLWGAASAVSVAGATVLLNILQMLVSYARKW |
| CYP4V3 GENE | QQMRPIPSVARAYPLVGHALFMKPNNTEFFQQIIQYTEEFRHLP |
| PRODUCT | IIKLWIGPVPLVALYKAENVEVILTSSKQIDKSFMYKFLQPWLG |
| NP_001129072.1 | LEKHVNQEAFNCFFPITLCALDIICETAMGKNIGAQSNGDSEYV |
| | RTVYRMSDMIYRRMKMPWFWFDLWYLMFKEGRDHKKGLKSLHTF |
| | TNNVIAERVNARKAEQDCIGAGRGPLPSKTKRKAFLDLLLSVTD |
| | EEGNKLSHEDIREEVDTFMFEGHDTTAAAINWSLYLLGSNPEVQ |
| | RKVDKELDDVFGRSHRPVTLEDLKKLKYLDCVIKETLRVFPSVP |
| | LFARSLSEDCEVAGYKISKGTEAVIIPYALHRDPRYFPDPEEFQ |
| | PERFFPENSQGRHPYAYVPFSAGPRNCIGQKFAVMEEKTILACI |
| | LREFWIESNQKREELGLAGDLILRPNNGIWIKLKRRHEDDP |
| Mus musculus | 45 |
| CYP4V3 CDS | ATGTTGTGGCTGTGGTTAGGGCTCAGTGGGCAGAAACTATTGCT |
| NM_133969.3 | TTGGGGCGCAGCGAGCGCGGTCTCCCTGGCCGGCGCCACTATCC |
| | TGATCAGCATCTTTCCGATGCTGGTAAGCTACGCGCGGAAATGG |
| | CAGCAGATGCGGTCAATCCCGTCGGTGGCCCGCGCCTACCCCTT |
| | GGTGGGACACGCGCTTTATATGAAGCCCAACAACGCAGAATTTT |
| | TTCAGCAGCTAATTTATTACACAGAAGAATTTCGACACCTGCCG |
| | ATCATTAAACTTTGGATTGGACCCGTGCCCCTGGTGGCACTTTA |
| | TAAGGCAGAGAATGTGGAGGTGATTTTGACCAGTTCTAAGCAAA |
| | TTGATAAATCGTTTTTGTACAAGTTCCTACAGCCATGGCTGGGA |
| | CTAGGACTTCTTACAAGTACGGGGAGCAAATGGCGCACCAGGAG |
| | GAAGATGCTAACGCCCACTTTCCATTTTACCATTCTGGAGAACT |
| | TCTTGGATGTCATGAATGAGCAAGCAAATATATTGGTTAATAAG |
| | CTTGAAAAACACGTCAACCAAGAAGCCTTTAATTGTTTTTTTTA |
| | CATCACTCTTTGTGCTCTGGATATAATCTGTGAAACGGCTATGG |
| | GGAAGAACATCGGAGCTCAAAGCAATAATGATTCCGAGTATGTC |
| | CGTACAGTGTATAGGATGAGTGATATGATATATAGAAGAATGAA |
| | GATGCCCTGGCTTTGGTTTGACCTTTGGTACCTTGTGTTTAAAG |
| | AAGGACGGGACCACAAAAGGGGACTCAAATGCCTACATACTTTC |
| | ACCAACAATGTCATTGCTGAACGAGTCAAAGAAAGGAAGGCAGA |
| | GGAAGACTGGACGGGTGCTGGCAGGGGTCCTATCCCCTCCAAAA |
| | ATAAGCGCAAGGCTTTCCTTGACTTGCTTTTGAGTGTGACTGAT |
| | GAGGAAGGAAACAGATTAAGCCAGGAAGACATCCGAGAGGAAGT |
| | TGACACCTTCATGTTTGAGGGTCACGATACAACTGCTGCTGCAA |
| | TCAACTGGTCCTTATACCTATTGGGCACGAATCCAGAAGTCCAG |
| | AGGAAAGTGGATCAGGAGCTGGATGAAGTGTTTGGAAGATCCCA |
| | TCGTCCTGTCACCTTGGAAGACCTGAAGAAACTTAAATATTTGG |
| | ATTGCGTCATTAAGGAGACTCTCCGAGTTTTCCCATCTGTCCCT |
| | TTATTTGCCCGGAGTCTTAGCGAGGACTGTGAAGTGGGCGGTTA |
| | CAAAGTCACAAAAGGAACGGAAGCAATCATCATTCCCTACGCAC |
| | TACACCGAGACCCCAGATACTTCCCAGATCCAGAGGAATTCCGA |
| | CCAGAGCGGTTCTTTCCTGAAAATTCCCAAGGACGCCATCCCTA |
| | TGCCTATGTGCCATTTTCTGCTGGACCTCGAAACTGTATTGGTC |
| | AAAAGTTTGCTGTCATGGAGGAGAAGACCATTCTTGCCTGTATC |
| | CTGAGGCAGTTTTGGGTAGAATCCAACCAGAAGAGAGAAGAACT |
| | CGGCCTGGCTGGAGATTTGATTCTTAGGCCAAATAATGGCATCT |
| | GGATCAAGCTGAAGAGGAGACATGAAGATGACCCCTAA |
| Mus musculus | 46 |
| CYP4V3 GENE | MLWLWLGLSGQKLLLWGAASAVSLAGATILISIFPMLVSYARKW |
| PRODUCT | QQMRSIPSVARAYPLVGHALYMKPNNAEFFQQLIYYTEEFRHLP |
| NP_598730.1 | IIKLWIGPVPLVALYKAENVEVILTSSKQIDKSFLYKFLQPWLG |
| | LGLLTSTGSKWRTRRKMLTPTFHFTILENFLDVMNEQANILVNK |
| | LEKHVNQEAFNCFFYITLCALDIICETAMGKNIGAQSNNDSEYV |
| | RTVYRMSDMIYRRMKMPWLWFDLWYLVFKEGRDHKRGLKCLHTF |
| | TNNVIAERVKERKAEEDWTGAGRGPIPSKNKRKAFLDLLLSVTD |
| | EEGNRLSQEDIREEVDTFMFEGHDTTAAAINWSLYLLGTNPEVQ |
| | RKVDQELDEVFGRSHRPVTLEDLKKLKYLDCVIKETLRVFPSVP |
| | LFARSLSEDCEVGGYKVTKGTEAIIIPYALHRDPRYFPDPEEFR |
| | PERFFPENSQGRHPYAYVPFSAGPRNCIGQKFAVMEEKTILACI |
| | LRQFWVESNQKREELGLAGDLILRPNNGIWIKLKRRHEDDP |
| Gallus gallus | 47 |
| CYP4V2 CDS | ATGGCAATGGAGATCACGCTAGGATCCATGGAGGGAACACAGCT |
| NM_001001879.1 | GCTGCCCTGGGTGGCTGGAGCCATCACCCTGCTGCTGACGGTGG |
| | TGACTGTACACTTCCTACCCTCTTTGCTGAACTACTGGTGGTGG |
| | TGGTGGGTGATGAAGCCCATCCCAGGCATCCGCCCATGCTACCC |
| | CTTTGTGGGAAATGCTCTCCTGTTGGAGCGAAATGGAGAAGGTT |
| | TTTTTAAACAGCTACAACAGTATGCTGATGAGTTCAGGAAAATG |
| | CCAATGTTCAAACTCTGGTTAGGTCCACTGCCTGTCACAGTATT |
| | GTTCCATCCTGATAGTGTGGAGGTTATTCTGAGCAGTTCAAAGC |
| | ATATTAAAAAATCATTCCTGTACACATTTCTGCACCCATGGCTG |
| | GGGACTGGACTTTTGACAAGCACTGGAGACAAGTGGCGGTCACG |
| | GAGGAAGATGATAACTCCTACATTCCACTTTGCAATCTTAAATG |
| | ACTTTCTTGAGGTTATGAATGAACAAGGGGGTGTTTTGTTGGAG |
| | AAACTTGAGAAGCATGTTGACAAGGAACCATTTAATATCTTTAC |
| | AGACATCACTCTGTGTGCACTTGATATTATCTGTGAAACTGCAA |
| | TGGGCAAGAATCTGGGTGCTCAAGACAATAAGGATTCTGAGTAT |
| | GTTCGTGCTGTCTACAGGATGAGTGATCTAATCCAACAGCGACA |
| | GAAGAGCCCTTGGCTTTGGCATGATCTTATGTATCTTCTGTTCA |
| | AGGAAGGAAGAGAGCATGAGCGGAATCTTAAGATTCTGCATGGT |
| | TTTACAGATACGGTAATTGCAGAAAAAGTTGCAGAACTTGAAAA |
| | CACCAAGCTAACAAAACACGATACTGACGTGAACACTGAAGAAG |
| | AAAGTGGTTCCAAAAAGAGAGAAGCTTTCTTAGACATGCTGCTG |
| | AATGCCACAGATGATGAAGGGAAAAAACTCAGCTACAAGGACAT |
| | TCGTGAAGAAGTGGATACTTTTATGTTTGAGGGTCATGATACAA |
| | CAGCAGCTGCTATGAACTGGGTCCTATACTTGCTTGGTCATCAT |
| | CCTGAAGCCCAGAAGAAGGTTCACCAAGAACTGGATGAGGTGTT |
| | TGGCAACACAGAGCGTCCTGTTACAGTGGATGATTTGAAGAAAC |
| | TTCGATACCTCGAGTGTGTTGTGAAAGAAGCCCTGAGGCTCTTC |
| | CCTTCAGTTCCCATGTTCGCCCGTTCCTTGCAAGAGGATTGCTA |
| | CATTAGTGGATATAAGCTACCAAAAGGCACGAATGTCCTTGTCT |
| | TAACTTATGTGCTGCACAGAGATCCTGAGATCTTCCCTGAGCCA |
| | GATGAATTCAGGCCTGAGCGCTTCTTCCCTGAAAATAGCAAAGG |
| | AAGGCACCCATATGCTTATGTGCCCTTCTCTGCTGGCCCCAGGA |
| | ACTGCATTGGCCAACGCTTTGCACAAATGGAAGAGAAAACTCTT |
| | CTAGCCCTCATCCTGCGGCGCTTTTGGGTGGACTGTTCTCAAAA |
| | GCCAGAAGAGCTTGGTCTGTCAGGAGAACTAATTCTTCGTCCAA |
| | ATAATGGCATCTGGGTTCAACTGAAGAGGAGACCAAAAACTGTA |
| | ACAGAATGA |
| Gallus gallus | 48 |
| CYP4V2 GENE | MAMEITLGSMEGTQLLPWVAGAITLLLTVVTVHFLPSLLNYWWW |
| PRODUCT | WWVMKPIPGIRPCYPFVGNALLLERNGEGFFKQLQQYADEFRKM |
| NP_001001879.1 | PMFKLWLGPLPVTVLFHPDSVEVILSSSKHIKKSFLYTFLHPWL |
| | GTGLLTSTGDKWRSRRKMITPTFHFAILNDFLEVMNEQGGVLLE |
| | KLEKHVDKEPFNIFTDITLCALDIICETAMGKNLGAQDNKDSEY |
| | VRAVYRMSDLIQQRQKSPWLWHDLMYLLFKEGREHERNLKILHG |
| | FTDTVIAEKVAELENTKLTKHDTDVNTEEESGSKKREAFLDMLL |
| | NATDDEGKKLSYKDIREEVDTFMFEGHDTTAAAMNWVLYLLGHH |
| | PEAQKKVHQELDEVFGNTERPVTVDDLKKLRYLECVVKEALRLF |
| | PSVPMFARSLQEDCYISGYKLPKGTNVLVLTYVLHRDPEIFPEP |
| | DEFRPERFFPENSKGRHPYAYVPFSAGPRNCIGQRFAQMEEKTL |
| | LALILRRFWVDCSQKPEELGLSGELILRPNNGIWVQLKRRPKTV |
| | TE |
| Canis lupus | 49 |
| familiaris | ATGTTAACACCCACTTTCCATTTTACGATTCTGGAAGATTTCTT |
| CYP4V2 CDS | AGATGTCATGAATGAACACGCAAATATATTGGTTAATAAGCTTG |
| XM_022404181.1 | AAAAACATGTTAACCAAGAAGCATTTAACTGCTTTTTTTACATC |
| | ACTCTTTGTGCATTAGATATAATTTGTGAAACAGCTATGGGGAA |
| | GAATATTGGGGCTCAAAATAATGAGGATTCTGAGTATGTTCGTG |
| | CCATCTACAGAATGAGTGATACGATACATCGAAGAATGAAGATG |
| | CCCTGGCTCTGGCTTGACTTTTTGTTTCTTATGTTTAAAGAAGG |
| | CCGGGAACACAAAAGGAACCTAGAGATCCTACATAATTTTACCA |
| | ATAATGTCATCACTGAACGGGCCAGTGAACTGAAGAGAGACGAA |
| | GAACATGGAAGTGCTGACAAGGACTGCTCCCCCTCCAAAAATAA |
| | ACGCAGAGCTTTTCTTGACTTGCTTTTAAATGTGACTGATGATG |
| | AAGGGAACAAGCTACGTCATGAAGATGTTCGAGAAGAAGTTGAC |
| | ACCTTCATGTTTGAGGGCCATGATACGACAGCAGCGGCGATAAA |
| | CTGGTCCTTATATCTCTTGGGTTCTTACCCAGAAGTCCAGAAAC |
| | AAGTGGACAGTGAACTGGAGGACGTGTTTGGGAAGTCTGATCGT |
| | CCTGCTACCTTAGAAGACCTGAAGAAACTCAAATACCTGGAGTG |
| | TGTCATTAAGGAGAGCCTTCGCCTTTTTCCTTCAGTTCCCTTAT |
| | TTGCCCGTAATCTTAACGAAGATTGTGTAGTTGCGGGTTACAAG |
| | GTTGTGAAAGGCTCCCAAGCGATCATCATTCCCTACGCACTTCA |
| | TAGAGATCCAAGATATTTCCCAAATCCCGAGGAGTTCCAGCCAG |
| | AGCGGTTCTTTCCTGAAAATTTGCAAGGACGCCACCCATATGCA |
| | TACATTCCCTTTTCTGCTGGACCCAGAAACTGTATAGGTCAAAG |
| | GTTTGCCATAATGGAAGAAAAGACTGTTCTTTCCTGTGTCCTGA |
| | GGCATTTTTGGGTAGAATCCAACCAGAAAAGAGAAGAACTTGGT |
| | CTGGCAGGAGAGTTGATTCTTCGTCCAACTAATGGCATCTGGAT |
| | CAAGTTGAAGAGGAGAAATGCAGATGAATCTTAA |
| Canis lupus | 50 |
| familiaris | MLTPTFHFTILEDFLDVMNEHANILVNKLEKHVNQEAFNCFFYI |
| CYP4V2 GENE | TLCALDIICETAMGKNIGAQNNEDSEYVRAIYRMSDTIHRRMKM |
| PRODUCT | PWLWLDFLFLMFKEGREHKRNLEILHNFTNNVITERASELKRDE |
| XP_022259889.1 | EHGSADKDCSPSKNKRRAFLDLLLNVTDDEGNKLRHEDVREEVD |
| | TFMFEGHDTTAAAINWSLYLLGSYPEVQKQVDSELEDVFGKSDR |
| | PATLEDLKKLKYLECVIKESLRLFPSVPLFARNLNEDCVVAGYK |
| | VVKGSQAIIIPYALHRDPRYFPNPEEFQPERFFPENLQGRHPYA |
| | YIPFSAGPRNCIGQRFAIMEEKTVLSCVLRHFWVESNQKREELG |
| | LAGELILRPTNGIWIKLKRRNADES |
| Kozak sequence | 51 |
| | GCCGCC |
| Kozak sequence | 52 |
| | GACACC |
| Kozak sequence | 53 |
| | GCCACG |

In some embodiments, the 5′ and 3′ ITRs comprise about 130 to about 145 nucleotides each. The ITRs are required for efficient multiplication of the AAV genome, and the symmetrical feature of these sequences gives them an ability to form a hairpin, which contributes to so-called self-priming that allows primase-independent synthesis of the second DNA strand. It is contemplated that the 5′ and 3′ ITRs of AAV serotype 2 may be used (e.g., nucleotide sequences with greater than or about 80%, 85%, 90%, 91%, 92%, 93%, 94%, 95%, 96%, 97%, 98%, 99%, or 100% identity to SEQ ID NOs:1 and 22, respectively). In other aspects, ITRs from other suitable serotypes may be selected from among any AAV serotype known in the art, as described herein, e.g., the ITRs may be from AAV8, AAV9, or AAV5.

These ITRs or other AAV components may be readily isolated using techniques available to those of skill in the art from any AAV serotype known, or yet to be identified serotypes, for example, the AAV sequences may be synthetic or obtained through other suitable means by reference to published sequences such as are available in the literature or in databases such as, e.g., GenBank, PubMed, or the like. Alternatively, such AAV components may also be isolated or obtained from academic, commercial, or public sources (e.g., the American Type Culture Collection, Manassas, Va.).

In some embodiments, the 5′ or 3′ ITR region of an AAV vector is mutated to form a ΔITR, e.g., by deleting/mutating the terminal resolution site (trs), and the resulting AAV genome becomes self-complementary (sc) by forming dimeric inverted repeat DNA molecules. In one embodiment, a ΔITR sequence comprises SEQ ID NO: 54. Additional ΔITR sequences are known in the art, e.g., as described in Wang et al., Gene Therapy, 2003, 10: 2105-2111; McCarty et al., Gene Therapy, 2003, 10: 2112-2118; and McCarty et al., Gene Therapy, 2001, 8: 1248-1254, each one of which is incorporated by reference in its entirety.

In certain embodiments, a RPE-specific promoter may be used to target expression of CYP4V2 preferentially in RPE cells, e.g., human RPE cells, of the retina. Examples of RPE-specific promoters include a ProA18 promoter or ProB4 promoter. For example, the ProA18 promoter can have a nucleotide sequence with greater than or about 80%, 85%, 90%, 91%, 92%, 93%, 94%, 95%, 96%, 97%, 98%, 99%, or 100% identity to SEQ ID NO:2, and the ProB4 promoter can have a nucleotide sequence with greater than or about 80%, 85%, 90%, 91%, 92%, 93%, 94%, 95%, 96%, 97%, 98%, 99%, or 100% identity to SEQ ID NO:3.

In some embodiments, the vector genome, e.g., single stranded vector genome, may comprise an intron sequence. For example, the intron may be a human growth hormone (hGH) intron, Simian Virus 40 (SV40) intron, or a human beta goblin intron, e.g., a nucleotide sequence with greater than or about 80%, 85%, 90%, 91%, 92%, 93%, 94%, 95%, 96%, 97%, 98%, 99%, or 100% identity to SEQ ID NO:9, 10, or 11, respectively.

In one embodiment, the vector genome, e.g., single stranded vector genome, comprises a recombinant nucleotide sequence comprising a CYP4V2 coding sequence. The CYP4V2 coding sequence can have a nucleotide sequence with greater than or about 80%, 85%, 90%, 91%, 92%, 93%, 94%, 95%, 96%, 97%, 98%, 99%, or 100% identity to SEQ ID NO:13, 14, 39, 41, 43, 45, 47, or 49. In one particular embodiment, the vector genome comprises a recombinant nucleotide sequence comprising a CYP4V2 coding sequence with greater than or about 80%, 85%, 90%, 91%, 92%, 93%, 94%, 95%, 96%, 97%, 98%, 99%, or 100% identity to SEQ ID NO:14.

In addition, the vector genome, e.g., single stranded vector genome, may include a regulatory element operably linked to the heterologous CYP4V2 gene. The regulatory element may include appropriate transcription initiation, termination, and enhancer sequences, efficient RNA processing signals such as splicing signals; sequences that stabilize cytoplasmic mRNA; sequences that enhance translation efficiency; sequences that enhance protein stability; and when desired, sequences that enhance secretion of the encoded product. A great number of regulatory sequences are known in the art and may be utilized. Regulatory element sequences of the invention include those described in Table 2, for example, Hepatitis B virus regulatory element (HPRE) and Woodchuck hepatitis virus regulatory element (WPRE), which can have a nucleotide sequence with greater than or about 80%, 85%, 90%, 91%, 92%, 93%, 94%, 95%, 96%, 97%, 98%, 99%, or 100% identity to SEQ ID NO:16 and 17, respectively.

In one embodiment, the vector genome, e.g., single stranded vector genome, comprises a polyA signal sequence. PolyA signal sequences of the invention include those described in Table 2, for example, Bovine Growth Hormone (bGH) polyA signal sequence and Simian Virus 40 (SV40) polyA signal sequence, which can have a nucleotide sequence with greater than or about 80%, 85%, 90%, 91%, 92%, 93%, 94%, 95%, 96%, 97%, 98%, 99%, or 100% identity to SEQ ID NO:18 and 19, respectively. In one particular embodiment, the vector genome comprises a bGH polyA signal sequence, which can have a nucleotide sequence with greater than or about 80%, 85%, 90%, 91%, 92%, 93%, 94%, 95%, 96%, 97%, 98%, 99%, or 100% identity to SEQ ID NO:18.

Therefore, in one aspect, the invention is related to a vector genome, e.g., single stranded vector genome, comprising, in the 5′ to 3′ direction: (i) a 5′ ITR (e.g., SEQ ID NO:1), (ii) a promoter (e.g., SEQ ID NO:2 or 3), (iii) a recombinant nucleotide sequence comprising a CYP4V2 coding sequence (e.g., SEQ ID NO:13, 14, 39, 41, 43, 45, 47, or 49), (iv) a polyA signal sequence (e.g., SEQ ID NO:18 or 19), and (v) a 3′ ITR (e.g., SEQ ID NO:22). In certain aspects of the invention, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) an intron (e.g., SEQ ID NO:9, 10, or 11), (iv) a recombinant nucleotide sequence comprising a CYP4V2 coding sequence (e.g., SEQ ID NO:13, 14, 39, 41, 43, 45, 47, or 49), (v) a polyA signal sequence (e.g., SEQ ID NO:18 or 19), and (vi) a 3′ ITR (e.g., SEQ ID NO:22). In some embodiments, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) a recombinant nucleotide sequence comprising a CYP4V2 coding sequence (e.g., SEQ ID NO:13, 14, 39, 41, 43, 45, 47, or 49), (iv) a regulatory element (e.g., SEQ ID NO:16 or 17), (v) a polyA signal sequence (e.g., SEQ ID NO:18 or 19), and (vi) a 3′ ITR (e.g., SEQ ID NO:22). In certain aspects of the invention, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) an intron (e.g., SEQ ID NO:9, 10, or 11), (iv) a recombinant nucleotide sequence comprising a CYP4V2 coding sequence (e.g., SEQ ID NO:13, 14, 39, 41, 43, 45, 47, or 49), (v) a regulatory element (e.g., SEQ ID NO:15 or 17), (vi) a polyA signal sequence (e.g., SEQ ID NO:18 or 19), and (vii) a 3′ ITR (e.g., SEQ ID NO:22).

In some embodiments, the vector genome, e.g., single stranded vector genome, may further comprise a stuffer polynucleotide sequence. The stuffer polynucleotide sequence can be located in the vector sequence at any desired position such that it does not prevent a function or activity of the vector. In one aspect, the stuffer polynucleotide sequence is positioned between the polyA signal sequence and the 3′ ITR. Typically, a stuffer polynucleotide sequence is inert or innocuous and has no function or activity. In various particular aspects, the stuffer polynucleotide sequence is not a bacterial polynucleotide sequence; the stuffer polynucleotide sequence is not a sequence that encodes a protein or peptide; and the stuffer polynucleotide sequence is distinct from an ITR sequence, the promoter, the recombinant nucleotide sequence comprising a CYP4V2 coding sequence, and the polyA signal sequence. In some embodiments, the stuffer sequence can be a nucleotide sequence with greater than or about 80%, 85%, 90%, 91%, 92%, 93%, 94%, 95%, 96%, 97%, 98%, 99%, or 100% identity to SEQ ID NO:20 or 21. In various additional aspects, a stuffer polynucleotide sequence is between about 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, or 2,500-3,000 nucleotides in length.

Therefore, in certain embodiments, the vector genome, e.g., single stranded vector genome, comprises, in the 5′ to 3′ direction: (i) a 5′ ITR (e.g., SEQ ID NO:1), (ii) a promoter (e.g., SEQ ID NO:2 or 3), (iii) a recombinant nucleotide sequence comprising a CYP4V2 coding sequence (e.g., SEQ ID NO:13, 14, 39, 41, 43, 45, 47, or 49), (iv) a polyA signal sequence (e.g., SEQ ID NO:18 or 19), (v) a stuffer sequence (e.g., SEQ ID NO:20 or 21), and (vi) a 3′ ITR (e.g., SEQ ID NO:22). In certain aspects of the invention, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) an intron (e.g., SEQ ID NO:9, 10, or 11), (iv) a recombinant nucleotide sequence comprising a CYP4V2 coding sequence (e.g., SEQ ID NO:13, 14, 39, 41, 43, 45, 47, or 49), (v) a polyA signal sequence (e.g., SEQ ID NO:18 or 19), (vi) a stuffer sequence (e.g., SEQ ID NO:20 or 21), and (vii) a 3′ ITR (e.g., SEQ ID NO:22). In some embodiments, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) a recombinant nucleotide sequence comprising a CYP4V2 coding sequence (e.g., SEQ ID NO:13, 14, 39, 41, 43, 45, 47, or 49), (iv) a regulatory element (e.g., SEQ ID NO:16 or 17), (v) a polyA signal sequence (e.g., SEQ ID NO:18 or 19), (vi) a stuffer sequence (e.g., SEQ ID NO:20 or 21), and (vii) a 3′ ITR (e.g., SEQ ID NO:22). In certain aspects of the invention, the vector genome, e.g., single stranded vector genome, comprises in the 5′ to 3′ direction: (i) a 5′ ITR (e.g., SEQ ID NO:1), (ii) a promoter (e.g., SEQ ID NO:2 or 3), (iii) an intron (e.g., SEQ ID NO:9, 10, or 11), (iv) a recombinant nucleotide sequence comprising a CYP4V2 coding sequence (e.g., SEQ ID NO:13, 14, 39, 41, 43, 45, 47, or 49), (v) a regulatory element (e.g., SEQ ID NO:16 or 17), (vi) a polyA signal sequence (e.g., SEQ ID NO:18 or 19), (vii) a stuffer sequence (e.g., SEQ ID NO:20 or 21), and (viii) a 3′ ITR (e.g., SEQ ID NO:22).

In some embodiments, the vector genome, e.g., single stranded vector genome, may also comprise a Kozak sequence. The Kozak sequence is a sequence that occurs on eukaryotic mRNA and has the consensus (gcc)gccRccAUGG sequence and plays a role in the initiation of the translation process. The Kozak sequence can be positioned immediately upstream of the recombinant nucleotide sequence comprising the CYP4V2 coding sequence. In some embodiments, the Kozak sequence is GCCACC (SEQ ID NO:12). Alternatively, the vector genome, e.g., single stranded vector genome, comprises a Kozak sequence of GCCGCC (SEQ ID NO:51), GACACC (SEQ ID NO:52), or GCCACG (SEQ ID NO:53).

In certain aspects of the invention, the viral vector comprises an AAV8 capsid comprising VP1, VP2, and VP3 amino acid sequences with greater than or about 80%, 85%, 90%, 91%, 92%, 93%, 94%, 95%, 96%, 97%, 98%, 99%, or 100% identity to SEQ ID NOs:24, 25, and 26, respectively, encoded by, for example, a nucleotide sequence with greater than or about 80%, 85%, 90%, 91%, 92%, 93%, 94%, 95%, 96%, 97%, 98%, 99%, or 100% identity to SEQ ID NO:23 and a vector genome comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the vector genome comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain embodiments, the AAV8 capsid may comprise subcombinations of capsid proteins VP1, VP2, and/or VP3.

It is also contemplated that the viral vector of the invention may comprise an AAV9 capsid comprising VP1, VP2, and VP3 amino acid sequences with greater than or about 80%, 85%, 90%, 91%, 92%, 93%, 94%, 95%, 96%, 97%, 98%, 99%, or 100% identity to SEQ ID NOs:28, 29, and 30, respectively, encoded by, for example, a nucleotide sequence with greater than or about 80%, 85%, 90%, 91%, 92%, 93%, 94%, 95%, 96%, 97%, 98%, 99%, or 100% identity to SEQ ID NO:27 and a vector genome comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the vector genome comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain embodiments, the AAV9 capsid may comprise subcombinations of capsid proteins VP1, VP2, and/or VP3.

In certain aspects of the invention, the viral vector comprises an AAV2 capsid comprising VP1, VP2, and VP3 amino acid sequences with greater than or about 80%, 85%, 90%, 91%, 92%, 93%, 94%, 95%, 96%, 97%, 98%, 99%, or 100% identity to SEQ ID NOs:32, 33, and 34, respectively, encoded by, for example, a nucleotide sequence with greater than or about 80%, 85%, 90%, 91%, 92%, 93%, 94%, 95%, 96%, 97%, 98%, 99%, or 100% identity to SEQ ID NO:31 and a vector genome comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the vector genome comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain embodiments, the AAV2 capsid may comprise subcombinations of capsid proteins VP1, VP2, and/or VP3.

In certain aspects of the invention, the viral vector comprises an AAV5 capsid comprising VP1, VP2, and VP3 amino acid sequences with greater than or about 80%, 85%, 90%, 91%, 92%, 93%, 94%, 95%, 96%, 97%, 98%, 99%, or 100% identity to SEQ ID NOs:36, 37, and 38, respectively, encoded by, for example, a nucleotide sequence with greater than or about 80%, 85%, 90%, 91%, 92%, 93%, 94%, 95%, 96%, 97%, 98%, 99%, or 100% identity to SEQ ID NO:35 and a vector genome comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the vector genome comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain embodiments, the AAV5 capsid may comprise subcombinations of capsid proteins VP1, VP2, and/or VP3.

Methods for generating viral vectors are well known in the art and would allow for the skilled artisan to generate the viral vectors of the invention (see, e.g., U.S. Pat. No. 7,465,583), including the viral vectors described in Table 3. In general, methods of producing rAAV vectors are applicable to producing the viral vectors of the invention; the primary difference between the methods is the structure of the genetic elements to be packaged. To produce a viral vector according to the present invention, sequences of the genetic elements described in Table 2 can be used to produce an encapsidated viral genome.

The genetic elements as described in Table 2 are in the context of a circular plasmid or a viral genome, e.g., a singled stranded or self-complementary viral genome, but one of skill in the art will appreciate that a DNA substrate may be provided in any form known in the art, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the genetic elements in Table 2 necessary to produce the viral vectors described herein may be stably incorporated into the genome of a packaging cell.

The viral vector particles according to the invention may be produced by any method known in the art, e.g., by introducing the sequences to be replicated and packaged into a permissive or packaging cell, as those terms are understood in the art (e.g., a “permissive” cell can be infected or transduced by the virus; a “packaging” cell is a stably transformed cell providing helper functions).

In one embodiment, a method is provided for producing a CYP4V2 viral vector, wherein the method comprises providing to a cell permissive for parvovirus replication: (a) a nucleotide sequence containing the genetic elements for producing a vector genome of the invention (as described in detail below and in Table 2); (b) nucleotide sequences sufficient for replication of the vector genome sequence in (a) to produce a vector genome; (c) nucleotide sequences sufficient to package the vector genome into a parvovirus capsid, under conditions sufficient for virus vectors comprising the vector genome encapsidated within the parvovirus capsid to be produced in the cell. Preferably, the parvovirus replication and/or capsid coding sequences are AAV sequences.

Any method of introducing the nucleotide sequence carrying the gene cassettes described below into a cellular host for replication and packaging may be employed, including but not limited to, electroporation, calcium phosphate precipitation, linear polyethylenimine polymer precipitation, microinjection, cationic or anionic liposomes, and liposomes in combination with a nuclear localization signal.

Viral vectors described herein may be produced using methods known in the art, such as, for example, triple transfection or baculovirus mediated virus production. Any suitable permissive or packaging cell known in the art may be employed to produce the vectors. Mammalian cells are preferred. Also preferred are trans-complementing packaging cell lines that provide functions deleted from a replication-defective helper virus, e.g., 293 cells or other E1a trans-complementing cells. Also preferred are mammalian cells or cell lines that are defective for DNA repair as known in the art, as these cell lines will be impaired in their ability to correct the mutations introduced into the plasmids described herein.

The gene cassette may contain some or all of the parvovirus (e.g., AAV) cap and rep genes. Preferably, however, some or all of the cap and rep functions are provided in trans by introducing a packaging vector(s) encoding the capsid and/or Rep proteins into the cell. Most preferably, the gene cassette does not encode the capsid or Rep proteins. Alternatively, a packaging cell line is used that is stably transformed to express the cap and/or rep genes (see, e.g., Gao et al., Hum Gene Ther 9:2353-2362, 1998; Inoue et al., J Virol 72:7024-7031, 1998; U.S. Pat. No. 5,837,484; WO 98/27207; U.S. Pat. No. 5,658,785; WO 96/17947).

In addition, helper virus functions are preferably provided for the virus vector to propagate new virus particles. Both adenovirus and herpes simplex virus may serve as helper viruses for AAV. See, e.g., Bernard N. Fields et al., VIROLOGY, volume 2, chapter 69 (3d ed., Lippincott-Raven Publishers). Exemplary helper viruses include, but are not limited to, Herpes simplex (HSV) varicella zoster, cytomegalovirus, and Epstein-Barr virus. The multiplicity of infection (MOI) and the duration of the infection will depend on the type of virus used and the packaging cell line employed. Any suitable helper vector may be employed. Preferably, the helper vector is a plasmid, for example, as described by Xiao et al., J Virol 72:2224, 1998. The vector can be introduced into the packaging cell by any suitable method known in the art, as described above.

Vector stocks free of contaminating helper virus may be obtained by any method known in the art. For example, recombinant single stranded or self complementary virus and helper virus may be readily differentiated based on size. The viruses may also be separated away from helper virus based on affinity for a heparin substrate (Zolotukhin et al., Gene Ther 6:973-985, 1999). Preferably, deleted replication-defective helper viruses are used so that any contaminating helper virus is not replication competent. As a further alternative, an adenovirus helper lacking late gene expression may be employed, as only adenovirus early gene expression is required to mediate packaging of the duplexed virus. Adenovirus mutants defective for late gene expression are known in the art (e.g., ts100K and ts149 adenovirus mutants).

One method for providing helper functions employs a non-infectious adenovirus miniplasmid that carries all of the helper genes required for efficient AAV production (Ferrari et al., Nat Med 3:1295-1297, 1997; Xiao et al., J Virol 72:2224-2232, 1998). The rAAV titers obtained with adenovirus miniplasmids are forty-fold higher than those obtained with conventional methods of wild-type adenovirus infection (Xiao et al., J Virol 72:2224-2232, 1998). This approach obviates the need to perform co-transfections with adenovirus (Hölscher et al., J Virol 68:7169-7177, 1994; Clark et al., Hum Gene Ther 6:1329-1341, 1995; Trempe and Yang, (1993), in, Fifth Parvovirus Workshop, Crystal River, Fla.).

Other methods of producing rAAV stocks have been described, including but not limited to, methods that split the rep and cap genes onto separate expression cassettes to prevent the generation of replication-competent AAV (see, e.g., Allen et al., J Virol 71:6816-6822, 1997), methods employing packaging cell lines (see, e.g., Gao et al., Hum Gene Ther 9:2353-2362, 1998; Inoue et al., J Virol 72:7024-7031, 1998; U.S. Pat. No. 5,837,484; WO 98/27207; U.S. Pat. No. 5,658,785; WO 96/17947), and other helper virus free systems (see, e.g., U.S. Pat. No. 5,945,335).

Herpesvirus may also be used as a helper virus in AAV packaging methods. Hybrid herpesviruses encoding the AAV Rep protein(s) may advantageously facilitate for more scalable AAV vector production schemes. A hybrid herpes simplex virus type I (HSV-1) vector expressing the AAV-2 rep and cap genes has been described (Conway et al., Gene Ther 6:986-993, 1999, and WO 00/17377).

In summary, the gene cassette to be replicated and packaged, parvovirus cap genes, appropriate parvovirus rep genes, and (preferably) helper functions are provided to a cell (e.g., a permissive or packaging cell) to produce rAAV particles carrying the vector genome. The combined expression of the rep and cap genes encoded by the gene cassette and/or the packaging vector(s) and/or the stably transformed packaging cell results in the production of a viral vector particle in which a viral vector capsid packages a viral vector genome according to the invention. The viral vectors, e.g., single stranded vectors, are allowed to assemble within the cell, and may then be recovered by any method known by those of skill in the art and described in the examples. For example, viral vectors may be purified by standard CsCl centrifugation methods (Grieger et al., Nat Protoc 1:1412-1428, 2006), iodixanol centrifugation methods, or by various methods of column chromatography known to the skilled artisan (see, e.g., Lock et al., Hum Gene Ther 21:1259-1271, 2010; Smith et al., Mol Ther 17:1888-1896, 2009; and Vandenberghe et al., Hum Gene Ther 21:1251-1257, 2010).

The reagents and methods disclosed herein may be employed to produce high-titer stocks of the inventive viral vectors, preferably at essentially wild-type titers. It is also preferred that the parvovirus stock has a titer of about 10¹⁰ vg/mL to about 10¹³ vg/mL, e.g., at least or about 10¹⁰ vg/mL, 6.6×10¹⁰ vg/mL, 10¹¹ vg/mL, 5×10¹¹ vg/mL, 10¹² vg/mL, 5×10¹² vg/mL, 10¹³ vg/mL, 5×10¹³ vg/mL, or more.

### Nucleic Acids for Use in Generating the Viral Vector

The invention also relates to nucleic acids useful for the generation of viral vectors. In certain aspects of the invention, the nucleic acids useful for the generation of viral vectors may be in the form of plasmids. Plasmids useful for the generation of viral vectors, also referred to as a viral vector plasmid, may contain a gene cassette. At a minimum, a gene cassette of a viral vector plasmid contains: a promoter, a heterologous CYP4V2 gene, a polyA signal sequence, and 5′ and 3′ ITRs.

The composition of the heterologous gene and other elements will depend upon the use to which the resulting vector will be put. For example, one type of heterologous gene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. For example, where the reporter sequence is the LacZ gene, the presence of the vector carrying the signal is detected by assays for beta-galactosidase activity. Where the reporter sequence is GFP or luciferase, the vector carrying the signal may be measured visually by color or light production in a luminometer.

The heterologous gene sequences, when associated with elements that drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry.

The heterologous gene may also be a non-marker sequence encoding a product that is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence that inhibits or extinguishes expression of a targeted nucleotide sequence in the treated animal.

The heterologous gene may also be used to correct or ameliorate gene deficiencies, which may include deficiencies in which normal genes are expressed at less than normal levels or deficiencies in which the functional gene product is not expressed. It is contemplated in the present invention that the heterologous gene sequence may be a CYP4V2 coding sequence. Examples of CYP4V2 coding sequences are provided in Table 2: SEQ ID NOs:13, 14, 39, 41, 43, 45, 47, and 49.

One aspect of the invention relates to nucleic acids that comprise a gene cassette comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the gene cassette comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain embodiments, the nucleic acid comprising the gene cassette may be a plasmid.

Methods for incorporating the elements in Table 2 are well known in the art and would allow for the skilled artisan to generate the nucleic acids and plasmids of the invention using the methods described herein.

### Pharmaceutical Compositions

In one aspect, the invention provides pharmaceutical compositions comprising the viral vectors of the invention formulated together with a pharmaceutically acceptable carrier. The compositions can additionally contain one or more other therapeutic agents that are suitable for treating or preventing BCD. Pharmaceutically acceptable carriers enhance or stabilize the composition, or can be used to facilitate preparation of the composition. Pharmaceutically acceptable carriers include solvents, surfactants, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be subretinal. The pharmaceutically acceptable carrier should be suitable for subretinal, intravitreal, intravenous, sub-cutaneous, or topical administration.

The composition should be sterile and fluid. Proper fluidity can be maintained, for example, by use of coating such as lecithin, by maintenance of required particle size in the case of dispersion, and by use of surfactants. In many cases, it is preferable to include isotonic agents, for example, sugars, polyalcohols such as mannitol or sorbitol, and sodium chloride in the composition. In one embodiment, the composition can include a buffer with 1×PBS and 0.001% PLURONIC™ F-68 as a surfactant, with a pH of about 6.5 to 8.0, e.g., pH 6.5 to 7.5 and pH 6.5 to 7.0.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the viral vector is employed in the pharmaceutical compositions of the invention. The viral vectors may be formulated into pharmaceutically acceptable dosage forms by conventional methods known to those of skill in the art. Dosage regimens are adjusted to provide the optimum desired response (e.g., a therapeutic response). For example, a single bolus may be administered, several doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient that is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, other drugs, compounds and/or materials used in combination with the particular compositions employed, the age, sex, weight, condition, general health and prior medical history of the patient being treated, and like factors.

A physician or veterinarian can start doses of the viral vectors of the invention employed in the pharmaceutical composition at levels lower than that required to achieve the desired therapeutic effect and gradually increase the dosage until the desired effect is achieved. In general, effective doses of the compositions of the present invention, for the treatment of BCD as described herein vary depending upon different factors, including means of administration, target site, physiological state of the patient, whether the patient is human or an animal, other medications administered, and whether treatment is prophylactic or therapeutic. Treatment dosages need to be titrated to optimize safety and efficacy. For subretinal administration with a viral vector, the dosage may range from about 1×10⁸ vector genomes (vg)/eye to about 1×10¹² vg/eye. For example, the dosage may be greater than or about 1×10⁸ vg/eye, 2.5×10⁸ vg/eye, 5×10⁸ vg/eye, 7.5×10⁸ vg/eye, 1×10⁹ vg/eye, 2.5×10⁹ vg/eye, 5×10⁹ vg/eye, 7.5×10⁹ vg/eye, 1×10¹⁰ vg/eye, 2.5×10¹⁰ vg/eye, 5×10¹⁰ vg/eye, 7.5×10¹⁰ vg/eye, 1×10¹¹ vg/eye, 2×10¹¹ vg/eye, 2.5×10¹¹ vg/eye, 5×10¹¹ vg/eye, 7.5×10¹¹ vg/eye, or 1×10¹² vg/eye.

The viral vectors described herein are mainly used as one time doses per eye, with the possibility of repeat dosing to treat regions of the retina that are not covered in the previous dosing. The dosage of administration may vary depending on whether the treatment is prophylactic or therapeutic.

The various features and embodiments of the present invention, referred to in individual sections and embodiments above apply, as appropriate, to other sections and embodiments, mutatis mutandis. Consequently, features specified in one section or embodiment may be combined with features specified in other sections or embodiments, as appropriate.

### Therapeutic Uses

Viral vectors as described herein, can be used at a therapeutically useful concentration for the treatment of eye related diseases, by administering to a subject in need thereof, an effective amount of the viral vectors of the invention. For example, the viral vector may comprises an AAV8 capsid comprising VP1, VP2, and VP3 amino acid sequences with greater than or about 80%, 85%, 90%, 91%, 92%, 93%, 94%, 95%, 96%, 97%, 98%, 99%, or 100% identity to SEQ ID NOs:24, 25, and 26, respectively, encoded by, for example, a nucleotide sequence with greater than or about 80%, 85%, 90%, 91%, 92%, 93%, 94%, 95%, 96%, 97%, 98%, 99%, or 100% identity to SEQ ID NO:23 and a vector genome comprising in the 5′ to 3′ direction nucleotide sequences with greater than or about 80%, 85%, 90%, 91%, 92%, 93%, 94%, 95%, 96%, 97%, 98%, 99%, or 100% identity to one of the following sets of nucleotide sequences:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

In some embodiments, the vector genome comprises in the 5′ to 3′ direction nucleotide sequences with greater than or about 80%, 85%, 90%, 91%, 92%, 93%, 94%, 95%, 96%, 97%, 98%, 99%, or 100% identity to SEQ ID NOs:1, 2, 14, 18, and 22; SEQ ID NOs:1, 2, 9, 14, 18, and 22; SEQ ID NOs:1, 2, 14, 16, 18, and 22; SEQ ID NOs:1, 2, 9, 14, 16, 18, and 22; SEQ ID NOs:1, 3, 14, 18, and 22; SEQ ID NOs:1, 3, 9, 14, 18, and 22; SEQ ID NOs:1, 3, 14, 16, 18, and 22; or SEQ ID NOs:1, 3, 9, 14, 16, 18, and 22. In certain other embodiments, the AAV8 capsid may comprise subcombinations of capsid proteins VP1, VP2, and/or VP3.

Subjects in need of treatment may include those who have one or more mutation in their CYP4V2 gene, e.g., Table 1. More specifically, the present invention provides a method of treating BCD, by administering to a subject in need thereof an effective amount of a viral vector comprising a CYP4V2 coding sequence (e.g., a nucleotide sequence encoding a human CYP4V2 protein, e.g., SEQ ID NO:15). In some aspects, provided herein is a method of improving vision in a subject with BCD, by administering to a subject in need thereof an effective amount of a viral vector comprising a CYP4V2 coding sequence (e.g., a nucleotide sequence encoding a human CYP4V2 protein, e.g., SEQ ID NO:15). In some aspects, provided herein is a method of preventing the decline of vision in a subject with BCD, by administering to a subject in need thereof an effective amount of a viral vector comprising a CYP4V2 coding sequence (e.g., a nucleotide sequence encoding a human CYP4V2 protein, e.g., SEQ ID NO:15). In specific aspects, the present invention provides viral vectors comprising a CYP4V2 coding sequence for use in treating BCD in a subject. In one embodiment, the viral vectors described herein can be administered subretinally or intravitreally using methods known to those of skill in the art. In one embodiment, the methods may include genotyping a subject to determine whether they possess one or more CYP4V2 mutation associated with BCD (see, e.g., Table 1), and treating a subject with one or more CYP4V2 mutation associated with BCD for BCD by administering a viral vector as described herein. In specific embodiments, a viral vector provided herein for use with methods of treating BCD comprises a CYP4V2 coding sequence (e.g., a nucleotide sequence encoding a human CYP4V2 protein, e.g., SEQ ID NO:15) operably linked to a promoter, e.g., a ProA18 or ProB4 promoter.

Use of recombinant AAV has been shown to be feasible and safe for the treatment of retinal disease. See, e.g., Bainbridge et al., N Engl J Med 358:2231-2239, 2008; Bainbridge et al., Gene Ther 15:1191-1192, 2008; Hauswirth et al., Hum Gene Ther 19:979-990, 2008; Maguire et al., N Engl J Med 358:2240-2248, 2008; Bennett et al., Lancet 388:661-672, 2016; and Russell et al., Lancet 390:849-860, 2017. The viral vectors described herein can be used, inter alia, to treat and prevent progression of BCD and reduce vision loss.

The present invention also relates to a method of expressing a CYP4V2 coding sequence in RPE cells by administering viral vectors of the invention to a subject in need thereof, e.g., a subject with one or more mutations in their CYP4V2 gene, e.g., Table 1. The present invention also relates to viral vectors of the invention for use in expressing a CYP4V2 coding sequence in RPE cells of the retina of the subject in need thereof. The invention also contemplates a method of delivering and expressing a CYP4V2 coding sequence to the retina, specifically to RPE cells in the retina, of a subject having BCD. It is contemplated that the CYP4V2 coding sequence is delivered to the subject in need thereof by contacting the retina and/or RPE cells of the subject (e.g., administering subretinally or intravitreally) with a viral vector as described herein. Alternatively, a CYP4V2 coding sequence is delivered to a subject by administering to the subject a viral vector as described herein.

In some aspects, the present invention further includes methods of expressing a CYP4V2 coding sequence in RPE cells in the retina of a subject having BCD, by contacting the retina of the subject with viral vectors of the invention. In certain aspects, RPE cells of the retina of the subject are contacted with viral vectors of the invention.

Treatment and/or prevention of ocular disease such as BCD can be determined by an ophthalmologist, optometrist, or health care professional using clinically relevant measurements of visual function, functional vision, retinal anatomy, and/or Quality of Life. Treatment of BCD means any action (e.g., administration of a viral vector described herein) contemplated to improve or preserve visual function, functional vision, retinal anatomy, and/or Quality of Life. In addition, prevention as it relates to BCD means any action (e.g., administration of a viral vector described herein) that inhibits, prevents, or slows a worsening in visual function, functional vision, retinal anatomy, and/or BCD phenotype, as defined herein, in a subject at risk for said worsening, e.g., decrease number and/or size of yellow or white crystalline-like deposits in the retina.

Visual function may include, for example, visual acuity, visual acuity with low illumination, visual field, central visual field, peripheral vision, contrast sensitivity, dark adaptation, photostress recovery, color discrimination, reading speed, dependence on assistive devices (e.g., large typeface, magnifying devices, telescopes), facial recognition, proficiency at operating a motor vehicle, ability to perform one or more activities of daily living, and/or patient-reported satisfaction related to visual function. Thus, in certain embodiments, treatment of BCD can be said to occur where a subject has at least a 10%, 20%, or 30% decrease or lack of a 10%, 20%, or 30% or more increase in time to a pre-specified degree of dark adaptation. In addition, treatment of BCD can be said to occur where a subject exhibits early severe night blindness and slow dark adaptation at a young age, followed by progressive loss of visual acuity, visual fields and color vision, leading to legal blindness, determined by a qualified health care professional, e.g., ophthalmologist and optometrist.

Exemplary measures of visual function include Snellen visual acuity, ETDRS visual acuity, low-luminance visual acuity, Amsler grid, Goldmann visual field, standard automated perimetry, microperimetry, Pelli-Robson charts, SKILL card, Ishihara color plates, Farnsworth D15 or D100 color test, standard electroretinography, multifocal electroretinography, validated tests for reading speed, facial recognition, driving simulations, and patient reported satisfaction. Thus, treatment of BCD can be said to be achieved upon a gain of or failure to lose 2 or more lines (or 10 letters) of vision on an ETDRS scale. In addition, in certain aspects, treatment of BCD can be said to occur upon improvement or slowing of loss of retinal function as measured by, for example, electroretinography; improvement or slowing of progression of retinal architecture as measured by, for example, optical coherence tomography (OCT); improvement or slowing of loss in ambulatory navigation, e.g., through a maze at various illumination intensities; and/or at least a 10%, 20%, or 30% increase or lack of 10%, 20%, or 30% decrease in reading speed (words per minute). In addition, in some aspects, treatment of BCD can be said to occur where a subject exhibits at least a 20% increase or lack of a 20% decrease in the proportion of correctly identified plates on an Ishihara test or correctly sequenced disks on a Farnsworth test. Thus, treatment of BCD can be determined by, for example, improvement of rate of dark adaptation, an improvement in visual acuity, or slowing the rate of visual acuity loss.

Undesirable aspects of retinal anatomy that may be treated or prevented include, for example, accumulation of small, yellow or white crystalline-like deposits of lipids in the retina, retinal atrophy, retinal pigment epithelium atrophy, narrowing of retinal vessels, pigmentary clumping, and subretinal fluid. Exemplary means of assessing retinal anatomy include fundoscopy, fundus photography, fluorescein angiography, indocyanine green angiography, OCT, spectral domain optical coherence tomography, scanning laser ophthalmoscopy, confocal microscopy, adaptive optics, fundus autofluorescence, biopsy, necropsy, and immunohistochemistry. Thus, the viral vectors described herein can be used to treat BCD in a subject, as determined by, for example, a reduction in the rate of development of retinal atrophy and/or a reduction in the number and/or size of yellow or white crystalline-like deposits in the retina.

Treatment of BCD can also be determined by, for example, improvement or preservation of Quality of Life. Skilled practioners will appreciate that Quality of Life can be determined by many different tests, e.g., National Eye Institute NEI-VFQ25 questionnaire.

Subjects to be treated with therapeutic agents of the present invention can also be administered other therapeutic agents or devices with known efficacy for treating retinal dystrophy such as vitamin and mineral preparations, low-vision aids, guide dogs, or other devices known to assist patients with low vision.

Currently, there are no other approved therapeutic agents for the treatment of BCD. As other new therapies emerge, the present compositions and newer therapies can be administered sequentially in either order or simultaneously as clinically indicated.

The following are exemplary embodiments of the present invention.

1. A viral vector comprising a vector genome comprising, in a 5′ to 3′ direction:

(i) a 5′ ITR;

(ii) a promoter;

(iii) a recombinant nucleotide sequence comprising a CYP4V2 coding sequence;

(iv) a polyadenylation (polyA) signal sequence; and

(v) a 3′ ITR.

2. The viral vector of embodiment 1, wherein the vector genome comprises, in the 5′ to 3′ direction:

(i) a 5′ ITR;

(ii) a promoter;

(iii) an intron;

(iv) a recombinant nucleotide sequence comprising a CYP4V2 coding sequence;

(v) a polyA signal sequence; and

(vi) a 3′ ITR.

3. The viral vector of embodiment 1, wherein the vector genome comprises, in the 5′ to 3′ direction:

(i) a 5′ ITR;

(ii) a promoter;

(iii) a recombinant nucleotide sequence comprising a CYP4V2 coding sequence;

(iv) a regulatory element;

(v) a polyA signal sequence; and

(vi) a 3′ ITR.

4. The viral vector of embodiment 1, wherein the vector genome comprises, in the 5′ to 3′ direction:

(i) a 5′ ITR;

(ii) a promoter;

(iii) an intron;

(iv) a recombinant nucleotide sequence comprising a CYP4V2 coding sequence;

(v) a regulatory element;

(vi) a polyA signal sequence; and

(vii) a 3′ ITR.

5. The viral vector of any one of embodiments 1 to 4, wherein the vector genome comprises a length greater than or about 4.1 kb and less than or about 4.9 kb.

6. The viral vector of any one of embodiments 1 to 5, wherein the vector genome comprises a stuffer sequence positioned between the polyA signal sequence and the 3′ ITR.

7. The viral vector of embodiment 6, wherein the stuffer sequence is between about 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, or 2,500-3,000 nucleotides in length.

8. The viral vector of any one of embodiments 1 to 7, wherein the 5′ ITR comprises a nucleotide sequence with greater than or about 90% identity to SEQ ID NO:1.

9. The viral vector of any one of embodiments 1 to 8, wherein the promoter is a retinal pigment epithelium (RPE)-specific promoter.

10. The viral vector of embodiment 9, wherein the promoter is a ProA18 promoter or ProB4 promoter.

11. The viral vector of embodiment 10, wherein the promoter comprises a nucleotide sequence with greater than or about 90% identity to SEQ ID NO:2 or SEQ ID NO:3, and promotes expression of the CYP4V2 in RPE cells.

12. The viral vector of any one of embodiments 1 to 11, wherein the CYP4V2 coding sequence comprises a nucleotide sequence with greater than or about 90% identity to SEQ ID NO:13, SEQ ID NO:14, SEQ ID NO:39, SEQ ID NO:41, SEQ ID NO:43, SEQ ID NO:45, SEQ ID NO:47, or SEQ ID NO:49.

13. The viral vector of any one of embodiments 1 to 12, wherein the polyA signal sequence comprises a bovine growth hormone or simian virus 40 polyA nucleotide sequence.

14. The viral vector of embodiment 13, wherein the polyA signal sequence comprises a nucleotide sequence with greater than or about 90% identity to SEQ ID NO:18 or SEQ ID NO:19.

15. The viral vector of any one of embodiments 1 to 14, wherein the 3′ ITR comprises a nucleotide sequence with greater than or about 90% identity to SEQ ID NO:22.

16. The viral vector of any one of embodiments 2 and 4, wherein the intron comprises a human growth hormone, simian virus 40, or human beta goblin intron sequence.

17. The viral vector of embodiment 16, wherein the intron comprises a nucleotide sequence with greater than or about 90% identity to SEQ ID NO:9, SEQ ID NO:10, or SEQ ID NO:11.

18. The viral vector of any one of embodiments 3 and 4, wherein the regulatory element comprises a hepatitis B virus or woodchuck hepatitis virus sequence.

19. The viral vector of embodiment 18, wherein the regulatory element comprises a nucleotide sequence with greater than or about 90% identity to SEQ ID NO:16 or SEQ ID NO:17.

20. The viral vector of any one of embodiments 1 to 19, wherein the vector genome comprises a Kozak sequence positioned immediately upstream of the recombinant nucleotide sequence comprising the CYP4V2 coding sequence.

21. The viral vector of embodiment 20, wherein the Kozak sequence comprises the nucleotide sequence of SEQ ID NO:12, SEQ ID NO:51, SEQ ID NO:52, or SEQ ID NO:53.

22. The viral vector of embodiment 1, wherein the vector genome comprises, in the 5′ to 3′ direction, nucleotide sequences selected from the group consisting of:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22; and

viii) SEQ ID NOs:1, 3, 14, 19, and 22.

23. The viral vector of embodiment 2, wherein the vector genome comprises, in the 5′ to 3′ direction, nucleotide sequences selected from the group consisting of:

i) SEQ ID NOs:1, 2, 9, 13, 18, and 22;

ii) SEQ ID NOs:1, 3, 9, 13, 18, and 22;

iii) SEQ ID NOs:1, 2, 9, 14, 18, and 22;

iv) SEQ ID NOs:1, 3, 9, 14, 18, and 22;

v) SEQ ID NOs:1, 2, 9, 13, 19, and 22;

vi) SEQ ID NOs:1, 3, 9, 13, 19, and 22;

vii) SEQ ID NOs:1, 2, 9, 14, 19, and 22; and

viii) SEQ ID NOs:1, 3, 9, 14, 19, and 22.

24. The viral vector of embodiment 3, wherein the vector genome comprises, in the 5′ to 3′ direction, nucleotide sequences selected from the group consisting of:

i) SEQ ID NOs:1, 2, 13, 16, 18, and 22;

ii) SEQ ID NOs:1, 3, 13, 16, 18, and 22;

iii) SEQ ID NOs:1, 2, 14, 16, 18, and 22;

iv) SEQ ID NOs:1, 3, 14, 16, 18, and 22;

v) SEQ ID NOs:1, 2, 13, 16, 19, and 22;

vi) SEQ ID NOs:1, 3, 13, 16, 19, and 22;

vii) SEQ ID NOs:1, 2, 14, 16, 19, and 22; and

viii) SEQ ID NOs:1, 3, 14, 16, 19, and 22.

25. The viral vector of embodiment 4, wherein the vector genome comprises, in the 5′ to 3′ direction, nucleotide sequences selected from the group consisting of:

i) SEQ ID NOs:1, 2, 9, 13, 16, 18, and 22;

ii) SEQ ID NOs:1, 3, 9, 13, 16, 18, and 22;

iii) SEQ ID NOs:1, 2, 9, 14, 16, 18, and 22;

iv) SEQ ID NOs:1, 3, 9, 14, 16, 18, and 22;

v) SEQ ID NOs:1, 2, 9, 13, 16, 19, and 22;

vi) SEQ ID NOs:1, 3, 9, 13, 16, 19, and 22;

vii) SEQ ID NOs:1, 2, 9, 14, 16, 19, and 22; and

viii) SEQ ID NOs:1, 3, 9, 14, 16, 19, and 22.

26. The viral vector of any one of embodiments 1 to 25, wherein the vector comprises an adeno-associated virus (AAV) serotype 8, 9, 2, or 5 capsid.

27. The viral vector of embodiment 26, wherein the AAV8 capsid comprises VP1, VP2, and VP3 amino acid sequences with greater than or about 90% identity to SEQ ID NOs:24, 25, and 26, respectively.

28. The viral vector of embodiment 26, wherein the AAV8 capsid is encoded by a nucleotide sequence with greater than or about 90% identity to SEQ ID NO:23.

29. The viral vector of embodiment 26, wherein the AAV9 capsid comprises VP1, VP2, and VP3 amino acid sequences with greater than or about 90% identity to SEQ ID NOs:28, 29, and 30, respectively.

30. The viral vector of embodiment 26, wherein the AAV9 capsid is encoded by a nucleotide sequence with greater than or about 90% identity to SEQ ID NO:27.

31. The viral vector of embodiment 26, wherein the AAV2 capsid comprises VP1, VP2, and VP3 amino acid sequences with greater than or about 90% identity to SEQ ID NOs:32, 33, and 34, respectively.

32. The viral vector of embodiment 26, wherein the AAV2 capsid is encoded by a nucleotide sequence with greater than or about 90% identity to SEQ ID NO:31.

33. The viral vector of embodiment 26, wherein the AAV5 capsid comprises VP1, VP2, and VP3 amino acid sequences with greater than or about 90% identity to SEQ ID NOs:36, 37, and 38, respectively.

34. The viral vector of embodiment 26, wherein the AAV5 capsid is encoded by a nucleotide sequence with greater than or about 90% identity to SEQ ID NO:35.

35. A composition comprising the viral vector of any of the preceding embodiments.

36. The composition of embodiment 35, wherein the composition further comprises a pharmaceutically acceptable excipient.

37. A method of expressing a heterologous CYP4V2 gene in a retinal cell, the method comprising contacting the retinal cell with the viral vector of any of the preceding embodiments.

38. The method of embodiment 37, wherein the retinal cell is a RPE cell.

39. A method of treating a subject with Bietti crystalline dystrophy (BCD), the method comprising administering to the subject an effective amount of the composition of embodiment 36.

40. A method of improving visual acuity, improving visual function or functional vision, or inhibiting decline of visual function or functional vision in a subject with BCD, the method comprising administering to the subject an effective amount of the composition of embodiment 36.

41. The composition of embodiment 36 for use in treating a subject with BCD.

42. The composition of embodiment 36 for use in improving visual acuity in a subject with BCD.

43. A nucleic acid comprising a gene cassette, wherein the gene cassette comprises, in the 5′ to 3′ direction:

(i) a 5′ ITR;

(ii) a promoter;

(iii) a recombinant nucleotide sequence comprising a CYP4V2 coding sequence;

(iv) a polyA signal sequence; and

(v) a 3′ ITR.

44. The nucleic acid of embodiment 43, wherein the nucleic acid is a plasmid.

45. The nucleic acid of embodiment 43, wherein the gene cassette comprises, in the 5′ to 3′ direction, nucleotide sequences selected from the group consisting of:

i) SEQ ID NOs:1, 2, 13, 18, and 22;

ii) SEQ ID NOs:1, 3, 13, 18, and 22;

iii) SEQ ID NOs:1, 2, 14, 18, and 22;

iv) SEQ ID NOs:1, 3, 14, 18, and 22;

v) SEQ ID NOs:1, 2, 13, 19, and 22;

vi) SEQ ID NOs:1, 3, 13, 19, and 22;

vii) SEQ ID NOs:1, 2, 14, 19, and 22;

viii) SEQ ID NOs:1, 3, 14, 19, and 22;

ix) SEQ ID NOs:1, 2, 9, 13, 18, and 22;

x) SEQ ID NOs:1, 3, 9, 13, 18, and 22;

xi) SEQ ID NOs:1, 2, 9, 14, 18, and 22;

xii) SEQ ID NOs:1, 3, 9, 14, 18, and 22;

xiii) SEQ ID NOs:1, 2, 9, 13, 19, and 22;

xiv) SEQ ID NOs:1, 3, 9, 13, 19, and 22;

xv) SEQ ID NOs:1, 2, 9, 14, 19, and 22;

xvi) SEQ ID NOs:1, 3, 9, 14, 19, and 22;

xvii) SEQ ID NOs:1, 2, 13, 16, 18, and 22;

xviii) SEQ ID NOs:1, 3, 13, 16, 18, and 22;

xix) SEQ ID NOs:1, 2, 14, 16, 18, and 22;

xx) SEQ ID NOs:1, 3, 14, 16, 18, and 22;

xxi) SEQ ID NOs:1, 2, 13, 16, 19, and 22;

xxii) SEQ ID NOs:1, 3, 13, 16, 19, and 22;

xxiii) SEQ ID NOs:1, 2, 14, 16, 19, and 22;

xxiv) SEQ ID NOs:1, 3, 14, 16, 19, and 22;

xxv) SEQ ID NOs:1, 2, 9, 13, 16, 18, and 22;

xxvi) SEQ ID NOs:1, 3, 9, 13, 16, 18, and 22;

xxvii) SEQ ID NOs:1, 2, 9, 14, 16, 18, and 22;

xxviii) SEQ ID NOs:1, 3, 9, 14, 16, 18, and 22;

xxix) SEQ ID NOs:1, 2, 9, 13, 16, 19, and 22;

xxx) SEQ ID NOs:1, 3, 9, 13, 16, 19, and 22;

xxxi) SEQ ID NOs:1, 2, 9, 14, 16, 19, and 22; and

xxxii) SEQ ID NOs:1, 3, 9, 14, 16, 19, and 22.

46. A viral vector comprising a vector genome comprising a promoter operably linked to a recombinant nucleotide sequence comprising a CYP4V2 coding sequence, wherein the promoter is a ProA18 promoter.

47. A viral vector comprising a vector genome comprising a promoter operably linked to a recombinant nucleotide sequence comprising a CYP4V2 coding sequence, wherein the promoter is a ProB4 promoter.

### EXAMPLES

The following examples are provided to further illustrate the invention but not to limit its scope. Other variants of the invention will be readily apparent to one of ordinary skill in the art and are encompassed by the appended claims.

### Example 1: Construction of AAV-ITR Plasmids

### 1.1. Cloning of AAV-ITR Plasmids:

The nucleotide sequences of the individual plasmid elements are described in Table 2. The sequences were either synthesized or purchased commercially. Table 3 describes the elements that exist in each plasmid that was constructed. Standard molecular biology cloning techniques were used in generating the plasmids described in Table 3. A plasmid backbone with kanamycin resistance was used as the backbone and starting material. The individual sequence elements were cloned in at restriction enzyme sites or using blunt end cloning.

Because the antibiotic resistance gene cassette contained in the plasmid backbone does not play a role in the production of the AAV vectors, one of skill in the art could use alternate plasmid backbones and/or antibiotic resistance gene cassettes and yield the same viral vectors.

1.2. Triple Plasmid Transfection to Produce rAAV Vectors:

Recombinant AAV (rAAV) viral vectors were generated by triple transfection methods. Methods for triple transfection are known in the art (Ferrari et al., Nat Med 3:1295-1297, 1997). Briefly, AAV-ITR-containing plasmids (described in Table 3), AAV-RepCap containing plasmid (carrying Rep2 and Cap8) and Adeno-helper plasmid (carrying genes that assist in completing AAV replication cycle) were co-transfected into 293 cells. Cells were cultured for 4 days. At the end of the culture period, the cells were lysed and the vectors in the culture supernatant and in the cell lysate were purified by column chromatography using AVB sepharose affinity columns (GE Healthcare Life Sciences). Skilled practioners will appreciate that a standard CsCl gradient centrifugation method (method based on Grieger et al., Nat Protoc 1:1412-1428, 2006) may also be used.

Alternatively, GMP-like rAAV vectors can be generated by the cell transfection and culture methods described above. The harvested cell culture material is then processed by column chromatography based on methods described by Lock et al., Hum Gene Ther 21:1259-1271, 2010; Smith et al., Mol Ther 17:1888-1896, 2009; and Vandenberghe et al., Hum Gene Ther 21:1251-1257, 2010.

TABLE 3
Plasmid Compositions

| | | |
|---|---|---|
| | SEQUENCE | |
| | IDENTIFIER | |
| Element | (SEQ ID NO:) | Cloning Strategy |

Plasmid NVS1 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProA18 Promoter | 2 | PCR amplify ProA18 |
| | | promoter adding MluI |
| | | and SacII restriction |
| | | sites. Ligate |
| | | SacII/MluI digested |
| | | amplicon into |
| | | SacII/MluI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII fragment |
| | | into XbaI/HindIII |
| | | sites of AAV plasmid |
| | | backbone. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |

Plasmid NVS2 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProA18 Promoter | 2 | PCR amplify promoter |
| | | adding MluI and SacII |
| | | restriction sites. |
| | | Ligate SacII/MluI |
| | | digested amplicon into |
| | | SacII/MluI cut AAV |
| | | plasmid backbone. |
| hGH intron | 9 | PCR amplify hGHintron |
| | | adding SacII and XbaI |
| | | restriction sites. |
| | | Ligate SacII/XbaI cut |
| | | amplicon into |
| | | SacII/XbaI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |

Plasmid NVS3 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProA18 Promoter | 2 | PCR amplify promoter |
| | | adding MluI and SacII |
| | | restriction sites. |
| | | Ligate SacII/MluI |
| | | digested amplicon into |
| | | SacII/MluI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| HPRE regulatory element | 16 | Digest plasmid |
| | | containing HPRE with |
| | | XhoI and blunt. Digest |
| | | with SalI. Ligate |
| | | into AAV plasmid cut |
| | | first with BglII, |
| | | blunted and cut with |
| | | SalI. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |

Plasmid NVS4 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProA18 Promoter | 2 | PCR amplify promoter |
| | | adding MluI and SacII |
| | | restriction sites. |
| | | Ligate SacII/MluI cut |
| | | amplicon into |
| | | SacII/MluI digested |
| | | AAV plasmid backbone. |
| hGH intron | 9 | PCR amplify hGHintron |
| | | adding SacII and XbaI |
| | | restriction sites. |
| | | Ligate SacII/XbaI cut |
| | | amplicon into |
| | | SacII/XbaI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| HPRE regulatory element | 16 | Cut plasmid containing |
| | | HPRE element with XhoI |
| | | then blunt ends and |
| | | cut with SalI. Digest |
| | | AAV plasmid backbone |
| | | with BglII then blunt |
| | | ends and cut with |
| | | SalI. Ligate. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI AAV |
| | | plasmid backbone |
| | | containing WT AAV2 |
| | | 5′ and 3′ ITRs. |

Plasmid NVS5 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProB4 Promoter | 3 | PCR amplify ProB4 |
| | | promoter adding MluI |
| | | and SacII restriction |
| | | sites. Ligate |
| | | SacII/MluI digested |
| | | amplicon into |
| | | SacII/MluI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII fragment |
| | | into XbaI/HindIII |
| | | sites of AAV plasmid |
| | | backbone. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| | | Plasmid NVS6 Composition |
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProB4 Promoter | 3 | PCR amplify promoter |
| | | adding MluI and SacII |
| | | restriction sites. |
| | | Ligate SacII/MluI |
| | | digested amplicon into |
| | | SacII/MluI cut AAV |
| | | plasmid backbone. |
| hGH intron | 9 | PCR amplify hGHintron |
| | | adding SacII and XbaI |
| | | restriction sites. |
| | | Ligate SacII/XbaI cut |
| | | amplicon into |
| | | SacII/XbaI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |

Plasmid NVS7 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProB4 Promoter | 3 | PCR amplify promoter |
| | | adding MluI and SacII |
| | | restriction sites. |
| | | Ligate SacII/MluI |
| | | digested amplicon into |
| | | SacII/MluI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| HPRE regulatory element | 16 | Digest plasmid |
| | | containing HPRE with |
| | | XhoI and blunt. Digest |
| | | with SalI. Ligate |
| | | into AAV plasmid cut |
| | | first with BglII, |
| | | blunted and cut with |
| | | SalI. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |

Plasmid NVS8 Composition

| | | |
|---|---|---|
| 5′ ITR | 1 | MluI and PmlI digested |
| | | AAV transfer plasmid |
| | | insert is ligated into |
| | | MluI and PmlI sites of |
| | | AAV plasmid backbone |
| | | containing WT AAV2 |
| | | 5′ and 3′ ITRs. |
| ProB4 Promoter | 3 | PCR amplify promoter |
| | | adding MluI and SacII |
| | | restriction sites. |
| | | Ligate SacII/MluI cut |
| | | amplicon into |
| | | SacII/MluI digested |
| | | AAV plasmid backbone. |
| hGH intron | 9 | PCR amplify hGHintron |
| | | adding SacII and XbaI |
| | | restriction sites. |
| | | Ligate SacII/XbaI cut |
| | | amplicon into |
| | | SacII/XbaI cut AAV |
| | | plasmid backbone. |
| CYP4V2 coding sequence | 14 | Cyp4V2 synthesis |
| | | fragment flanked by |
| | | XbaI and HindIII |
| | | sites. Ligate |
| | | XbaI/HindIII cut |
| | | fragment into |
| | | XbaI/HindIII sites of |
| | | AAV plasmid backbone. |
| HPRE regulatory element | 16 | Cut plasmid containing |
| | | HPRE element with XhoI |
| | | then blunt ends and |
| | | cut with SalI. Digest |
| | | AAV plasmid backbone |
| | | with BglII then blunt |
| | | ends and cut with |
| | | SalI. Ligate. |
| bGH polyA signal sequence | 18 | Ligate HindIII/SgrA1 |
| | | fragment containing |
| | | bGH polyA signal |
| | | sequence into AAV |
| | | plasmid digested with |
| | | HindIII/SgrAI. |
| 3′ ITR | 22 | MluI and PmlI digested |
| | | AAV transfer plasmid |
| | | insert is ligated into |
| | | MluI and PmlI AAV |
| | | plasmid backbone |
| | | containing WT AAV2 |
| | | 5′ and 3′ ITRs. |

### Example 2: Inclusion of the hGH Intron, bGH polyA Signal Sequence, and HPRE Provide Enhanced Expression of eGFP

AAV8 vectors expressing ChR2d-eGFP were designed containing different elements, including different introns, different polyA signal sequences, and with or without a HPRE, to determine the best combination for optimal gene expression in the retina.

### Methods

C57BL/6 mice were injected subretinally with 1×10⁹ vg of AAV8 vectors expressing channelrhodopsin fused to eGFP (ChR2d-eGFP) and containing different elements, e.g., different introns (e.g., hGH intron and SV40 intron), polyA signal sequences (e.g., bGH polyA signal sequence and SV40 polyA signal sequence), and with or without a HPRE. Four weeks later, eyes were harvested from the mice and some were fixed with 1 ml 4% paraformaldehyde (PFA) fixative at 4° C. overnight and then placed in phosphate-buffered saline (PBS). These eyes were then dried with a paper towel and transferred to a 35 mm dish. Extraneous tissue was removed from the outside of the eye, as well as the cornea and lens, and then the eyecup was submerged in 1 ml PBS. The retina was then removed from the eyecup, and both were cut into petals and mounted on slides. eGFP fluorescent images were obtained using a Zeiss Axio Imager M1 fluorescent microscope and AxioVision software. All images were taken at 2.5× magnification and with the same exposure time.

The other harvested eyes were separated into neural retina and posterior eyecup and frozen for analysis of ChR2d-eGFP mRNA expression using droplet digital PCR (ddPCR). Briefly, RNA was isolated from the tissue samples using the Qiagen RNeasy Mini Kit and then 200 ng RNA was used to generate cDNA using the High-Capacity cDNA Reverse Transcription Kit from ThermoFisher Scientific. 1 ng of cDNA for each sample was added to ddPCR Supermix and primers and probe that recognize eGFP (Mr04097229_mr; ThermoFisher Scientific) and mouse Rab7 (Mm00784318_sH; ThermoFisher Scientific) were added to each reaction. ddPCR was performed according to the manufacturer's protocol (Bio-Rad). ChR2d-eGFP expression was normalized to Rab7 control expression for each sample.

### Results and Conclusions

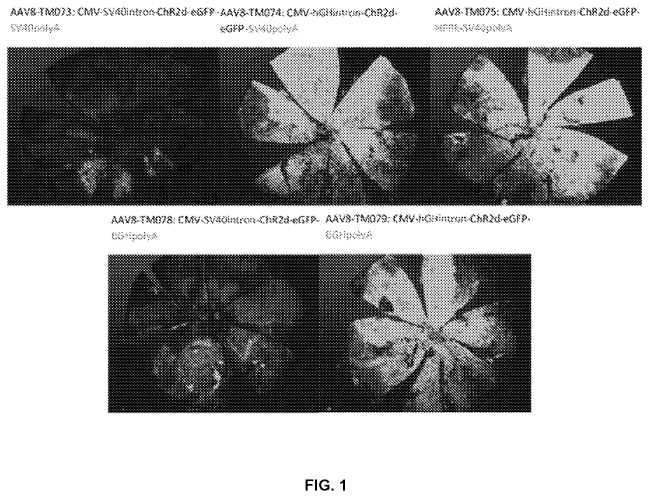

Five different AAV8 vectors were constructed and injected subretinally into mice. Four weeks post-injection, eyes were harvested from the mice and ChR2d-eGFP expression was examined by both flatmount and ddPCR. Flatmounts of the posterior eyecup showed eGFP fluorescence was highest in the eyes injected with vectors containing the hGH intron (FIG. 1). Also, inclusion of the bGH polyA signal sequence showed slightly higher fluorescence compared to the same vector with the SV40 polyA signal sequence (AAV8-TM078 versus AAV8-TM073).

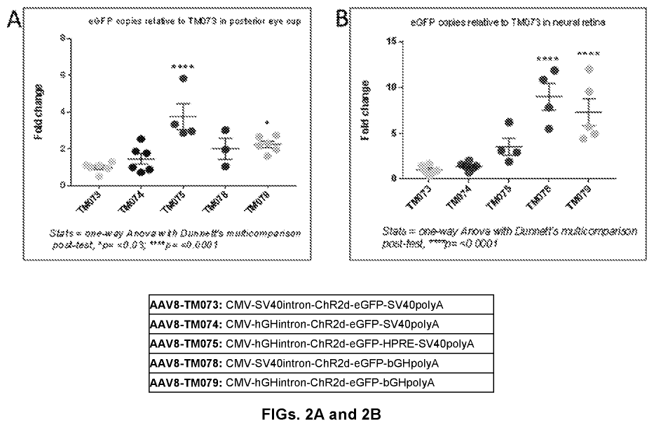

To obtain a more quantitative analysis of ChR2d-eGFP expression level, ddPCR was performed on mRNA isolated from separated neural retina and posterior eyecup samples. The results showed that in the posterior eyecup, expression was most significantly enhanced by addition of the HPRE (AAV8-TM075) (FIG. 2A). Also, vectors containing the bGH polyA signal sequence showed higher expression compared to the respective vectors containing the SV40 polyA signal sequence (compare AAV8-TM078 to AAV8-TM073, and AAV8-TM079 to AAV8-TM074). In the neural retina, addition of the HPRE again led to an enhancement in expression level of eGFP (AAV8-TM075), but the greatest increase in expression was observed from addition of the bGH polyA signal sequence (AAV8-TM078 and AAV8-TM079) (FIG. 2B).

Taken together, these results show that the optimal expression cassette for gene expression in the retina may include the hGH intron, bGH polyA signal sequence, and HPRE elements. Therefore, vectors containing one, two, or all three of those elements were constructed for delivery of the CYP4V2 cDNA for the treatment of BCD.

### Example 3: Subretinal Injection of AAV-CYP4V2 Vectors into Cyp4v3 Knockout Mice Prevents Progression of Disease

Cyp4v3 is the mouse ortholog of human CYP4V2 (82% identity). The Cyp4v3 gene was knocked out using CRISPR/Cas9, and Cyp4v3 knockout mice are injected with AAV vector expressing CYP4V2 at approximately 1×10⁹ vg per eye. At different time points post-injection, mice are examined by fundus imaging and optical coherence tomography to determine the number and size of crystalline deposits present in the retina. In addition, the mice are evaluated for visual function (e.g., visual acuity, dark adaptation) and functional vision (e.g., mobility test). Compared to control-injected eyes (AAV-eGFP vector), eyes injected with the vector expressing CYP4V2 show a reduction in number and/or size of crystalline deposits and/or an improvement in visual function and/or functional vision, demonstrating successful expression of CYP4V2 in the RPE cells and restoration of CYP4V2 protein function.

### Example 4: Subretinal Injection of AAV-ProA18 or AAV-ProB4 Vectors into Non-Human Primates Leads to Gene Expression in RPE Cells

Synthetic promoter sequences were chemically synthesized by GENEWIZ, with short flanks containing MluI/NheI/AscI and BamHI/EcoRI/BgIII restriction sites. ProB18 and ProB4 promoter sequences were subcloned using an appropriate restriction site combination into pAAV-EF1a-CatCh-GFP replacing the EF1a or hRO promoters. The pAAV-EF1a-CatCh-GFP plasmid was constructed by adapter PCR and the Clontech In-Fusion kit using pcDNA3.1(−)-CatCh-GFP.

HEK293T cells were co-transfected with an AAV transgene plasmid, an AAV helper plasmid encoding the AAV Rep2 and Cap proteins for the selected capsid (BP2), and the pHGT1-Adenol helper plasmid harboring the adenoviral genes using branched polyethyleneimine (Polysciences). One cell culture dish 15 cm in diameter was co-transfected with the plasmid mixture at 80% confluence of the HEK293T cells. A cell transfection mixture containing 7 pg AAV transgene plasmid, 7 pg Rep2 and Cap-encoding plasmid, 20 pg AAV helper plasmid and 6.8 μM polyethyleneimine in 5 ml of DMEM was incubated at room temperature for 15 min before being added to a cell culture dish containing 10 ml of DMEM. At 60 h post-transfection, cells were collected and resuspended in buffer containing 150 mM NaCl and 20 mM Tris-HCl, pH 8.0. Cells were lysed by repeated freeze—thaw cycles and MgCl2 was added to make a final concentration of 1 mM. Plasmid and genomic DNA were removed by treatment with 250 U ml-1 of TurboNuclease at 37° C. for 10 min. Cell debris was removed by centrifugation at 4,000 r.p.m. for 30 min. AAV particles were purified and concentrated in Millipore Amicon 100 K columns (catalog no. UFC910008; Merck Millipore). Encapsidated viral DNA was quantified by TaqMan reverse transcription PCR following denaturation of the AAV particles using protease K; titers were calculated as genome copies per ml.

For AAV administration in mice, ocular injections were performed on mice anesthetized with 2.5% isoflurane. A small incision was made with a sharp 30-G needle in the sclera near the lens and 2 μl of AAV suspension was injected through this incision into the subretinal/intravitreal space using a blunt 5-μl Hamilton syringe (Hamilton Company) held in a micromanipulator.

For AAV administration in non-human primates, 50 microliter of AAV particle suspension were injected subretinally in collaboration with an ophthalmologist and a third party contractor in Kunming, China. After 3 month, the isolated eyecups were fixed overnight in 4% PFA in PBS, followed by a washing step in PBS at 4 C. After receiving the fixed eyecups, the infected retinal region was dissected out and treated with 10% normal donkey serum (NDS), 1% BSA, 0.5% Triton X-100 in PBS for 1h at room temperature. Treatment with monoclonal rat anti-GFP Ab (Molecular Probes Inc.; 1:500) and polyclonal goat anti-ChAT (Millipore: 1:200) in 3% NDS, 1% BSA, 0.5% Triton X-100 in PBS was carried out for 5 days at room temperature. Treatment with secondary donkey anti-rat Alexa Fluor-488 Ab (Molecular Probes Inc.; 1:200), anti-goat Alexa Fluor-633 and Hoechst, was done for 2 hr. Sections were washed, mounted with ProLong Gold antifade reagent (Molecular Probes Inc.) on glass slides, and photographed using a Zeiss LSM 700 Axio Imager Z2 laser scanning confocal microscope (Carl Zeiss Inc.).

Tables 4 and 5 below summarize the ability of the synthetic promoters ProA18 and ProB4 to drive expression in mouse and NHP retinal cells. Both ProA18 and ProB4 lead to gene expression in RPE cells in NHP.

TABLE 4
Cell Specificity Expression in Mouse and NHP Retinal Cells (ProA18)

| | Targeted Cell Types | | | | |
|---|---|---|---|---|---|
| | | | Targeted cell | | |
| | | | density as a | | |
| | | | percentage of | Target | |
| | | | target | Expression | |
| | In order of | Targeting | population | Outer | Inner |
| | abundance | specificity | density | Retina | Retina |
| Mouse | MG | MG (100%) | MG (11 ± | 0 | 1 |
| | | | 2.7%) | | |
| NHP | RPE | RPE (100%) | RPE (79 ± | | |
| | | | 9.2%) | | |

TABLE 5
Cell Specificity Expression in Mouse and NHP Retinal Cells (ProB4)

| | Targeted Cell Types | | | | |
|---|---|---|---|---|---|
| | | | Targeted cell | | |
| | | | density as a | | |
| | | | percentage of | Target | |
| | | | target | Expression | |
| | In order of | Targeting | population | Outer | Inner |
| | abundance | specificity | density | Retina | Retina |
| Mouse | OFF BC, s-C | OFF BC | OFF BC (28 ± | 1 | 1 |
| | | (76%), C | 5.8%) | | |
| | | (24%) | | | |
| NHP | RPE | RPE (100%) | RPE (50 ± | | |
| | | | 4.6%) | | |

MG = Müller glia;
C = cones;
s-(as prefix) = sparse expression;
RPE = retinal pigment epithelium cells;
BC = Bipolar cells

### Example 5: Subretinal Injection of AAV Vectors into Cynomolgus Monkeys to Determine the Best Capsid Serotype for Targeting RPE Cells

AAV2, AAV6, AAV8, and AAV9 vectors expressing GFP from a CMV promoter were injected subretinally in cynomolgus monkeys at a dose of 1×10¹¹ vg per eye. Four weeks post-injection, GFP expression in the monkey eyes was examined in-life by fundus autofluorescence imaging and post-harvest by immunohistochemistry. In addition, the level and localization of GFP mRNA and AAV genomic DNA were assessed by in-situ hybridization.

All four serotypes tested facilitated GFP expression in photoreceptor and RPE cells. Expression was observed near the injection site with varying degree of spread to peripheral regions. GFP expression was not detected in optic nerve or brain sections for any of the serotypes tested.

### Example 6: Subretinal Injection of AAV Vectors into Cynomolgus Monkeys to Determine the Best Promoter for RPE-Specific Expression

AAV vectors expressing GFP from RPE-specific promoters are injected subretinally in cynomolgus monkeys at a dose of 1×10¹¹ vg per eye. The RPE-specific promoters include ProA18 and ProB4. Four weeks post-injection, GFP expression in the monkey eyes is examined in-life by fundus autofluorescence imaging and post-harvest by immunohistochemistry. In addition, the level and localization of GFP mRNA and AAV genomic DNA are assessed by in-situ hybridization. The two different promoters both show RPE-specific GFP expression but the level of expression is variable.

### Example 7: AAV-Driven Gene Expression in Human Retinal Tissues

Human retinal tissues are prepared from enucleated human eyeballs from which the retina is dissected suing fine scissors. AAV vectors expressing GFP from RPE-specific promoters are incubated with human retinal tissues. The RPE-specific promoters include ProA18 and ProB4. AAV-induced GFP expression is examined 6-8 weeks after virus administration. Six weeks post-injection, GFP expression in the human retinal tissues are examined by via immunofluorescence and imaging. The two different promoters both show RPE-specific GFP expression.

### Example 8: AAV-Driven Expression of CYP4V2 Under the ProA18 or ProB4 Promoter in NHP and Human Tissues

AAV vectors expressing human CYP4V2 under the ProA18 or ProB4 promoter (“AAV-ProA18-CYP4V2 vector” or “AAV-ProB4-CYP4V2 vector”) are incubated with human retinal tissues as described in Example 7. CYP4V2 gene expression is examined 6-8 weeks after virus administration. Six weeks post-injection, CYP4V2 expression in the human retinal tissues is detected.

AAV-ProA18-CYP4V2 or AAV-ProB4-CYP4V2 vector is injected subretinally in cynomolgus monkeys at a dose of 1×10¹¹ vg per eye. Four weeks post-injection, CYP4V2 expression in the monkey eyes is examined post-harvest by immunohistochemistry. In addition, the level and localization of CYP4V2 mRNA and AAV genomic DNA are assessed by in-situ hybridization. The ProA18 and ProB4 promoter induce RPE-specific expression of the CYP4V2 gene in the monkey eyes.

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples. Any references cited herein, including, e.g., all patents, published patent applications, and non-patent publications, are incorporated by reference in their entirety.

What is claimed is:

1. A viral vector comprising a vector genome comprising, in a 5′ to 3′ direction:
(i) a 5′ ITR;
(ii) a promoter;
(iii) a recombinant nucleotide sequence comprising a CYP4V2 coding sequence;
(iv) a polyadenylation (polyA) signal sequence; and
(v) a 3′ ITR.

2. The viral vector of claim 1, wherein the promoter is a retinal pigment epithelium (RPE)-specific promoter.

3. The viral vector of claim 1 or 2, wherein the promoter is a ProA18 promoter.

4. The viral vector of claim 1 or 2, wherein the promoter comprises a nucleotide sequence with greater than or about 90% identity to SEQ ID NO: 2.

5. The viral vector of claim 1 or 2, wherein the promoter is a ProB4 promoter.

6. The viral vector of claim 1 or 2, wherein the promoter comprises a nucleotide sequence with greater than or about 90% identity to SEQ ID NO: 3.

7. The viral vector of any one of claims 1 to 6, wherein the vector genome comprises a stuffer sequence positioned between the polyA signal sequence and the 3′ ITR.

8. The viral vector of any one of claims 1 to 7, wherein the 5′ ITR comprises a nucleotide sequence with greater than or about 90% identity to SEQ ID NO: 1.

9. The viral vector of any one of claims 1 to 8, wherein the CYP4V2 coding sequence comprises a nucleotide sequence with greater than or about 90% identity to SEQ ID NO: 13, 14, 39, 41, 43, 45, 47, or 49.

10. The viral vector of any one of claims 1 to 9, wherein the polyA signal comprises a nucleotide sequence with greater than or about 90% identity to SEQ ID NO: 18 or 19.

11. The viral vector of any one of claims 1 to 10, further comprises an intron sequence comprising a nucleotide sequence with greater than or about 90% identity to SEQ ID NO: 9, 10, or 11.

12. The viral vector of any one of claims 1 to 11, further comprises 1) a regulatory element comprising a hepatitis B virus or woodchuck hepatitis virus sequence and/or 2) a Kozak sequence positioned immediately upstream of the recombinant nucleotide sequence comprising the CYP4V2 coding sequence.

13. The viral vector of any one of claims 1 to 12, wherein the vector genome comprises, in the 5′ to 3′ direction, nucleotide sequences selected from the group consisting of:
i) SEQ ID NOs:1, 2, 13, 18, and 22;
ii) SEQ ID NOs:1, 3, 13, 18, and 22;
iii) SEQ ID NOs:1, 2, 14, 18, and 22;
iv) SEQ ID NOs:1, 3, 14, 18, and 22;
v) SEQ ID NOs:1, 2, 13, 19, and 22;
vi) SEQ ID NOs:1, 3, 13, 19, and 22;
vii) SEQ ID NOs:1, 2, 14, 19, and 22;
viii) SEQ ID NOs:1, 3, 14, 19, and 22;
ix) SEQ ID NOs:1, 2, 9, 13, 18, and 22;
x) SEQ ID NOs:1, 3, 9, 13, 18, and 22;
xi) SEQ ID NOs:1, 2, 9, 14, 18, and 22;
xii) SEQ ID NOs:1, 3, 9, 14, 18, and 22;
xiii) SEQ ID NOs:1, 2, 9, 13, 19, and 22;
xiv) SEQ ID NOs:1, 3, 9, 13, 19, and 22;
xv) SEQ ID NOs:1, 2, 9, 14, 19, and 22;
xvi) SEQ ID NOs:1, 3, 9, 14, 19, and 22;
xvii) SEQ ID NOs:1, 2, 13, 16, 18, and 22;
xviii) SEQ ID NOs:1, 3, 13, 16, 18, and 22;
xix) SEQ ID NOs:1, 2, 14, 16, 18, and 22;
xx) SEQ ID NOs:1, 3, 14, 16, 18, and 22;
xxi) SEQ ID NOs:1, 2, 13, 16, 19, and 22;
xxii) SEQ ID NOs:1, 3, 13, 16, 19, and 22;
xxiii) SEQ ID NOs:1, 2, 14, 16, 19, and 22;
xxiv) SEQ ID NOs:1, 3, 14, 16, 19, and 22;
xxv) SEQ ID NOs:1, 2, 9, 13, 16, 18, and 22;
xxvi) SEQ ID NOs:1, 3, 9, 13, 16, 18, and 22;
xxvii) SEQ ID NOs:1, 2, 9, 14, 16, 18, and 22;
xxviii) SEQ ID NOs:1, 3, 9, 14, 16, 18, and 22;
xxix) SEQ ID NOs:1, 2, 9, 13, 16, 19, and 22;
xxx) SEQ ID NOs:1, 3, 9, 13, 16, 19, and 22;
xxxi) SEQ ID NOs:1, 2, 9, 14, 16, 19, and 22; and
xxxii) SEQ ID NOs:1, 3, 9, 14, 16, 19, and 22.

14. The viral vector of any one of claims 1 to 13, further comprises an adeno-associated virus (AAV) serotype 8, 9, 2, or 5 capsid.

15. The viral vector of claim 14, further comprises 1) an AAV9 capsid comprising VP1, VP2, and VP3 amino acid sequences with greater than or about 90% identity to SEQ ID NOs: 28, 29, and 30, respectively; or 2) an AAV9 capsid encoded by a nucleotide sequence with greater than or about 90% identity to SEQ ID NO: 27.

16. The viral vector of claim 14, further comprises 1) an AAV8 capsid comprising VP1, VP2, and VP3 amino acid sequences with greater than or about 90% identity to SEQ ID NOs: 24, 25, and 26, respectively; or 2) an AAV8 capsid encoded by a nucleotide sequence with greater than or about 90% identity to SEQ ID NO: 23.

17. A composition comprising the viral vector of any one of claims 1 to 16.

18. A method of expressing a heterologous CYP4V2 gene in a retinal cell, the method comprising contacting the retinal cell with the viral vector of any one of claims 1 to 16.

19. A method of treating a subject with Bietti crystalline dystrophy (BCD), the method comprising administering to the subject an effective amount of the composition of claim 17.

20. A method of improving visual acuity, improving visual function or functional vision, or inhibiting decline of visual function or functional vision in a subject with BCD, the method comprising administering to the subject an effective amount of the composition of claim 17.
